(19) United States
(12) Patent Application Publication
(10) Pub. No.: US 2023/0301890 A1
(43) Pub. Date: Sep. 28, 2023

(54) COMPOSITIONS COMPRISING UROLITHINS

(71) Applicant: Amazentis SA, Lausanne (CH)

(72) Inventors: Christopher L. Rinsch, Lausanne (CH); Anurag Singh, Lausanne (CH); Davide D'Amico, Renens (CH)

(21) Appl. No.: 18/123,143

(22) Filed: Mar. 17, 2023

(30) Foreign Application Priority Data
Mar. 22, 2022 (GB) 2203966,3
Nov. 8, 2022 (GB) 2216648,2

(51) Int. Cl. A61K 8/49; A61K 8/9789; A61K 8/67; A61K 8/60; A61Q 19/08

(52) U.S. Cl. CPC A61K 8/498; A61K 8/9789; A61K 8/673; A61K 8/4953; A61K 8/60; A61Q 19/08

(57) ABSTRACT

Disclosed are compositions, particularly topical compositions comprising a urolithin, cosmetic compositions comprising a urolithin and cosmetic compositions comprising a urolithin and trehalose, compositions comprising urolithin and niacinamide (nicotinamide) and compositions comprising urolithin, trehalose, niacinamide and caffeine. Furthermore, the invention relates to urolithin for use in improving skin barrier function, for example, maintaining skin hydration, improving skin elasticity and firmness, and/or increasing or maintaining skin layer thickness.

### RELATED APPLICATIONS

This application claims the benefit of priority to Great Britain (UK) Patent Application Serial Nos. 2216648.2, filed Nov. 8, 2022; and 2203966.3, Mar. 22, 2022.

### BACKGROUND

The major function of the skin is to form a barrier between the internal milieu of a host and environmental insults, such as chemicals, ultraviolet light, mechanical insults and pathogenic microorganisms. The skin's structure is made up of layers of cells and cellular-derived tissue, with the outermost set of layers being the epidermis. The surface layer is called the stratum comeum then, proceeding inwards: the stratum granulosum, stratum spinosum, stratum basale, and then the dermis layer. In addition, the palms of the hands and the soles of the feet have an additional layer, the stratum lucidum, between the stratum corneum and the stratum granulosum. Numerous products have been developed to improve the functioning and/or appearance of skin and hair.

For example, in 2016, the beauty and personal care market was close to a 500-billion-dollar industry. Repeated exposure to environmental insults, such as the sun, causes skin thinning, fragility and wrinkles. More serious sun exposure can cause a loss of skin elasticity, deep wrinkles, increased roughness and dryness, and altered pigmentation (age spots, skin spots). The skin can also become leathery, thickened in appearance, which is characterized by deep furrows. Other environmental insults, such as chemicals causing burns and/or injury, acne or viral infection (e.g., chicken pox) can cause scaring, resulting in more serious and permanent damage to the skin.

Additionally, diseases can also cause changes in the skin's appearance. For example, Lupus patients often exhibit a characteristic rash, terms a “butterfly rash” that spans the face of the patient.

Additionally, the robustness and integrity of the skin, particularly with respect to its ability to respond to environmental stresses and/or resist disease, generally declines with age.

Urolithins have been proposed as treatments for a variety of conditions related to inadequate mitochondrial activity, including obesity, reduced metabolic rate, metabolic syndrome, diabetes mellitus, cardiovascular disease, hyperlipidaemia, neurodegenerative diseases, cognitive disorders, mood disorders, stress, and anxiety disorders; for weight management, or to increase muscle performance or mental performance. See WO 2012/088519 (Amazentis SA).

International patent publication WO 2014/004902 (derived from application PCT/US2013/48310) discloses a method of increasing autophagy, including specifically mitophagy, in a cell, comprising contacting a cell with an effective amount of a urolithin or a pharmaceutically acceptable salt thereof, thereby increasing autophagy, including specifically mitophagy, in the cell. Administration may be to a subject having a disease or condition selected from metabolic stress, cardiovascular disease, endothelial cell dysfunction, sarcopenia, muscle degenerative disease, Duchenne muscular dystrophy, alcoholic liver disease, non-alcoholic fatty liver disease, drug-induced liver or muscle injury, al-antitrypsin deficiency, ischemia/reperfusion injury, inflammation, aging of the skin, inflammatory bowel disease, Crohn's disease, obesity, metabolic syndrome, type II diabetes mellitus, hyperlipidaemia, osteoarthritis, neurodegenerative disease, Alzheimer's disease, Huntington's disease, Parkinson's disease, amyotrophic lateral sclerosis, age-related macular degeneration, mitochondrial diseases (including for example poor growth, loss of muscle coordination, muscle weakness, visual problems, hearing problems, heart disease, liver disease, kidney disease, gastrointestinal disorders, respiratory disorders, neurological problems, autonomic dysfunction sometimes learning disabilities, and dementia (as a result of mitochondrial disease), muscle diseases; cancer, cognitive disorder, stress, and mood disorder.

There are numerous different skin care formulations that are marketed as improving the appearance of the skin regardless of the type of skin insult. Examples of such formulations include, but are not limited to retinols, hydroxyl acids, Coenzyme Q₁₀, copper peptides, Kinetin, and/or tea extracts. Even though numerous creams, oils, topicals and oral products are launched every month, there is an ongoing need for new regimens that are safe and effective in improving the functioning of a subject's largest organ—the skin. For example, although retinoids show promise in the treatment of skin aging, irritant reactions such as burning, scaling or dermatitis associated with retinoid therapy limit their acceptance by patients. Some uses of urolithins have been disclosed with respect to skin, however, we have found improved formulations and combinations for use in skin, particularly for cosmetic use and novel uses of urolithins for the improvement of skin barrier function.

### SUMMARY

The invention relates to compositions, particularly topical compositions comprising a urolithin, cosmetic compositions comprising a urolithin and cosmetic compositions comprising a urolithin and trehalose, compositions comprising urolithin and niacinamide (nicotinamide) and compositions comprising urolithin, trehalose, niacinamide and caffeine. Furthermore, the invention relates to urolithin for use in improving skin barrier function, for example, maintaining skin hydration, improving skin elasticity and firmness, and/or increasing or maintaining skin layer thickness.

(1) Compositions Comprising Trehalose and/or an NAD Precursor

The present invention provides a composition comprising:
a) trehalose; and
b) a compound of formula (I) or a salt thereof:

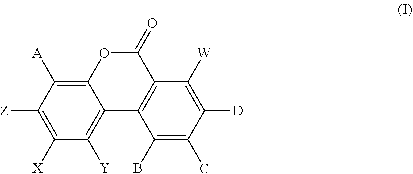

wherein:
A, B, C and D are each independently selected from H and OH;
W, X and Y are each independently selected from H and OH; and
Z is selected from H and OH.

In a further embodiment of the invention there is provided a composition comprising niacinamide (nicotinamide) and a compound of formula (I), or a salt thereof, as described above.

In a further embodiment of the invention there is provided a composition comprising an NAD precursor and a compound of formula (I), or a salt thereof, as described above wherein the NAD precursor is selected from nicotinic acid (niacin), niacinamide (nicotinamide), tryptophan, nicotinamide mononucleotide and nicotinamide riboside.

In a further embodiment of the invention there is provided a composition comprising trehalose, niacinamide (nicotinamide) and a compound of formula (I), or a salt thereof, as described above.

In a further embodiment, there is provided a composition comprising trehalose, an NAD precursor and a compound of formula (I), or a salt thereof, as described above.

In a further embodiment, there is provided a composition of the invention comprising a compound of formula (I) and trehalose and/or an NAD precursor, for example, a cream or lotion, comprising about 0.8% to about 2.0% of a compound of formula (I), for example, urolithin A.

In a further embodiment, there is provided a composition of the invention comprising a compound of formula (I) and trehalose and/or an NAD precursor, for example, a cream or lotion, comprising about 0.8% to about 1.5% of a compound of formula (I), for example, urolithin A.

In a further embodiment, there is provided a composition of the invention comprising a compound of formula (I) and trehalose and/or an NAD precursor, for example, a cream or lotion, comprising about 0.8% to about 1.2% of a compound of formula (I), for example, urolithin A.

In a further embodiment, there is provided a composition of the invention comprising a compound of formula (I) and trehalose and/or an NAD precursor, for example, a cream or lotion, comprising about 1% of a compound of formula (I), for example, urolithin A.

In a further embodiment, there is provided a composition of the invention comprising a compound of formula (I) and trehalose and/or an NAD precursor, for use in the treatment of one or more indications, described herein.

In a further embodiment, there is provided a composition of the invention comprising a compound of formula (I) and trehalose and/or an NAD precursor, for use as a cosmetic.

In a further embodiment there is provided a method of cosmetically treating skin using a composition of the invention comprising a compound of formula (I) and trehalose and/or an NAD precursor.

A method of cosmetic treatment of skin using an effective amount of a cosmetic composition comprising a compound of formula (I) and trehalose and/or an NAD precursor.

Use of a compound of formula (I) and trehalose and/or an NAD precursor as a cosmetic product.

Non-therapeutic use of cosmetic preparations comprising a compound of formula (I) and trehalose and/or an NAD precursor.

In a further embodiment, there is provided a composition of the invention comprising:
a) a compound of formula (I), for example, urolithin A;
b) Trehalose; and
c) an NAD precursor, for example, Niacinamide.

In a further embodiment, there is provided a composition of the invention comprising:
a) a compound of formula (I), for example, urolithin A;
b) Trehalose;
c) an NAD precursor, for example, Niacinamide, and
d) caffeine.

In a further embodiment, there is provided a composition of the invention comprising:
a) a compound of formula (I), for example, urolithin A;
b) Trehalose;
c) an NAD precursor, for example, Niacinamide, and
d) a B vitamin, for example, D-Panthenol.

In a further embodiment, there is provided a composition of the invention comprising:
a) a compound of formula (I), for example, urolithin A;
b) Trehalose;
c) an NAD precursor, for example, Niacinamide, and
d) Nopa flower extract (Opuntia ficus-indica).

In a further embodiment, there is provided a composition of the invention comprising:
a) a compound of formula (I), for example, urolithin A;
b) Trehalose;
c) an NAD precursor, for example, Niacinamide, and
d) saccharide isomerate.

In a further embodiment, there is provided a composition of the invention comprising:
a) a compound of formula (I), for example, urolithin A;
b) Trehalose;
c) an NAD precursor, for example, Niacinamide, and
d) Nicotiana sylvestris leaf cell culture.

In a further embodiment, there is provided a composition of the invention comprising:
a) a compound of formula (I), for example, urolithin A;
b) Trehalose;
c) an NAD precursor, for example, Niacinamide,
d) Nicotiana sylvestris leaf cell culture; and
e) saccharide isomerate.

In a further embodiment, there is provided a composition of the invention comprising:
a) a compound of formula (I), for example, urolithin A;
b) Trehalose;
c) an NAD precursor, for example, Niacinamide,
d) Nopa flower extract (Opuntia ficus-indica);
e) Caffeine;
f) Saccharide isomerate, and
g) a B vitamin, for example, D-Panthenol.

In a further embodiment, there is provided a composition of the invention comprising:
a) a compound of formula (I), for example, urolithin A;
b) Trehalose;
c) an NAD precursor, for example, Niacinamide,
d) Caffeine; and
e) a B vitamin, for example, D-Panthenol.

In a further embodiment, there is provided a composition of the invention comprising:
a) a compound of formula (I), for example, urolithin A
   wherein the compound of formula (I) comprises 0.8% to 5% (w/w) of the composition;
b) Trehalose,
   wherein the trehalose comprises 0.05% to 6% (w/w) of the composition; and
c) an NAD precursor, for example, niacinamide,
   wherein the niacinamide comprises 0.1% to 10% (w/w) of the composition.

In a further embodiment, there is provided a composition of the invention comprising:
a) a compound of formula (I), for example, urolithin A
   wherein the compound of formula (I) comprises 0.8% to 4% (w/w) of the composition
b) Trehalose,
   wherein the trehalose comprises 0.1% to 0.8% (w/w) of the composition; and
c) an NAD precursor, for example, niacinamide,
   wherein the niacinamide comprises 0.5% to 8% (w/w) of the composition.

In a further embodiment, there is provided a composition of the invention comprising:
a) a compound of formula (I), for example, urolithin A
   wherein the compound of formula (I) comprises 0.8% to 3% (w/w) of the composition
b) Trehalose,
   wherein the trehalose comprises 0.1% to 0.7% (w/w) of the composition; and
c) an NAD precursor, for example, niacinamide,
   wherein the niacinamide comprises 0.5% to 6% (w/w) of the composition.

In a further embodiment, there is provided a composition of the invention comprising:
a) a compound of formula (I), for example, urolithin A
   wherein the compound of formula (I) comprises 0.8% to 2% (w/w) of the composition
b) Trehalose,
   wherein the trehalose comprises 0.1% to 0.5% (w/w) of the composition; and
c) an NAD precursor, for example, niacinamide,
   wherein the niacinamide comprises 0.5% to 5% (w/w) of the composition.

In a further embodiment, there is provided a composition of the invention comprising:
a) a compound of formula (I), for example, urolithin A
   wherein the compound of formula (I) comprises about 1% (w/w) of the composition
b) Trehalose,
   wherein the trehalose comprises about 0.25% (w/w) of the composition; and
c) an NAD precursor, for example, niacinamide,
   wherein the niacinamide comprises about 1% to 3% (w/w) of the composition.

In a further embodiment, there is provided a composition of the invention comprising:
a) a compound of formula (I), for example, urolithin A
   wherein the compound of formula (I) comprises 0.8% to 5% (w/w) of the composition
b) Trehalose,
   wherein the trehalose comprises 0.05% to 1% (w/w) of the composition;
c) an NAD precursor, for example, niacinamide,
   wherein the niacinamide comprising 0.1% to 10% (w/w) of the composition; and
d) An anti-oxidant/promoter of blood circulation, for example, caffeine,
   wherein the anti-oxidant/promoter of blood circulation, for example, caffeine comprises 0.1% to 5% (w/w) of the composition.

In a further embodiment, there is provided a composition of the invention comprising:
a) a compound of formula (I), for example, urolithin A
   wherein the compound of formula (I) comprises 0.8% to 4% (w/w) of the composition
b) Trehalose,
   wherein the trehalose comprises 0.1% to 0.8% (w/w) of the composition; and
c) an NAD precursor, for example, niacinamide,
   wherein the niacinamide comprises 0.5% to 8% (w/w) of the composition.
d) An anti-oxidant/promoter of blood circulation, for example, caffeine
   wherein the anti-oxidant/promoter of blood circulation, for example, caffeine comprises 0.1% to 3% (w/w) of the composition.

In a further embodiment, there is provided a composition of the invention comprising:
a) a compound of formula (I), for example, urolithin A
   wherein the compound of formula (I) comprises 0.8% to 3% (w/w) of the composition
b) Trehalose,
   wherein the trehalose comprises 0.1% to 0.7% (w/w) of the composition; and
c) an NAD precursor, for example, niacinamide,
   wherein the niacinamide comprises 0.5% to 6% (w/w) of the composition.
d) An anti-oxidant/promoter of blood circulation, for example, caffeine
   wherein the anti-oxidant/promoter of blood circulation, for example, caffeine comprises 0.1% to 2% (w/w) of the composition.

In a further embodiment, there is provided a composition of the invention comprising:
a) a compound of formula (I), for example, urolithin A
   wherein the compound of formula (I) comprises 0.8% to 2% (w/w) of the composition
b) Trehalose,
   wherein the trehalose comprises 0.1% to 0.5% (w/w) of the composition; and
c) an NAD precursor, for example, niacinamide,
   wherein the niacinamide comprises 0.5% to 5% (w/w) of the composition.
d) An anti-oxidant/promoter of blood circulation, for example, caffeine
   wherein the anti-oxidant/promoter of blood circulation, for example, caffeine comprises 0.1% to 1% (w/w) of the composition.

In a further embodiment, there is provided a composition of the invention comprising:
a) a compound of formula (I), for example, urolithin A
   wherein the compound of formula (I) comprises about 1% (w/w) of the composition
b) Trehalose,
   wherein the trehalose comprises about 0.25% (w/w) of the composition; and
c) an NAD precursor, for example, niacinamide,
   wherein the niacinamide comprises about 1% to 3% (w/w) of the composition.
d) An anti-oxidant/promoter of blood circulation, for example, caffeine
   wherein the anti-oxidant/promoter of blood circulation, for example, caffeine comprises about 0.5% (w/w) of the composition.

Examples of anti-oxidants/promoters of blood circulations include caffeine, Green Tea (Camellia sinensis leaf extract), vitamin C, vitamin K and vitamin E. In a further embodiments, compositions of the invention comprise vitamin C in the following amounts, about 0.1% to about 5% (w/w) of the composition, about 0.5% to about 2% (w/w), about 0.7% to 1.5% (w/w) or about 1%. In a further embodiments, compositions of the invention comprise vitamin K in the following amounts, about 0.5% to about 10% (w/w) of the composition, about 1% to about 8% (w/w), about 2% to 7% (w/w), about 4% to about 6% (w/w) or about 5% (w/w). In a further embodiments, compositions of the invention comprise vitamin E in the following amounts, about 0.5% to about 10% (w/w) of the composition, about 1% to about 8% (w/w), about 2% to 7% (w/w), about 4% to about 6% (w/w) or about 5% (w/w).

In a further embodiment, compositions of the invention comprise a compound of Formula (1) in the following amounts: about 0.8% to about 5% (w/w), about 0.8% to about 4% (w/w), about 0.8% to about 3% (w/w), about 0.8% to about 2% (w/w), about 0.8% to about 1% (w/w), or about 1% (w/w).

In a further embodiment, compositions of the invention comprise trehalose in the following amounts: about 0.05% to about 8% (w/w), about 0.1% to about 6% (w/w), about 0.1% to about 5% (w/w), about 0.1% to about 4% (w/w), about 0.1% to about 3% (w/w), about 0.1% (w/w) to about 2% (w/w), about 0.1% to about 0.9% (w/w), about 0.1% to about 0.8% (w/w), about 0.1% to about 0.7% (w/w), about 0.1% to about 0.5% (w/w), about 0.1% to about 0.4% (w/w), about 0.1% to about 0.35% (w/w), about 0.2% to about 0.3% (w/w), or about 0.25% (w/w), or about 0.5% (w/w), about 1% (w/w), about 2% (w/w), about 3% (w/w), about 4% (w/w) or about 5% (w/w).

In a further embodiment, compositions of the invention comprise an NAD precursor in the following amounts: about 0.1% to about 10% (w/w), 0.25% to about 9% (w/w), 0.25% to about 8% (w/w), 0.5% to about 7% (w/w), 0.5% to about 6% (w/w), 0.5% to about 5% (w/w), 0.5% to about 4% (w/w), 1% to about 4% (w/w), 1% to about 3% (w/w), about 1% (w/w), about 2% (w/w), about 3% (w/w), about 4% (w/w) or about 5% (w/w).

In a further embodiment, compositions of the invention comprise an anti-oxidant/promoter of blood circulation, for example, caffeine, in the following amounts: about 0.1% to about 5% (w/w), about 0.1% to about 4% (w/w), about 0.1% to about 4% (w/w), about 0.1% to about 4% (w/w), about 0.1% to about 3% (w/w), about 0.1% to about 2% (w/w), about 0.1% to about 1% (w/w), about 0.2% to about 0.9% (w/w), about 0.3% to about 0.8% (w/w), about 0.3% to about 0.7% (w/w), about 0.3% to about 0.6% (w/w), about 0.4% to about 0.6% (w/w) or about 0.5% (w/w)

In a further embodiment, compositions of the invention comprise a B vitamin, for example, D-Panthenol, in the following amounts, about 0.1% to about 5% (w/w), about 0.1% to about 4% (w/w), about 0.1% to about 3% (w/w), about 0.1% to about 2% (w/w), about 0.1% to about 2% (w/w), about 1% (w/w) or about 0.5% (w/w).

In a further embodiment, compositions of the invention comprise Nopa flower extract (Opuntia ficus-indica), in the following amounts, about 0.05% to about 0.2% (w/w), about 0.05% to about 0.15% (w/w), about 0.075 to about 0.125% (w/w), about 0.075 to about 0.1% (w/w), about 0.1% (w/w). The Nopa flower extract can comprise one or more of the following: hydrolyzed Opuntia ficus-indica flower extract, Opuntia ficus-indica stem extract and Opuntia ficus-indica callus culture extract.

In a further embodiment, compositions of the invention comprise saccharide isomerate, in the following amounts, about 0.5% to about 5% (w/w), about 0.5% to about 4% (w/w), 0.5% to about 3% (w/w), 0.5% to about 2% (w/w), about 0.825% (w/w), about 1.65% (w/w) or about 2.75% (w/w).

In a further embodiment, compositions of the invention comprise Nicotiana sylvestris leaf cell culture, in the following amounts, about 0.0005% to about 0.005% (w/w), about 0.0005% to about 0.004% (w/w), about 0.0005% to about 0.004% (w/w), about 0.0005% to about 0.003% (w/w), about 0.001% to about 0.002% (w/w) or about 0.0015% (w/w).

### (2) Compositions Comprising a Compound of Formula (1), a Further Autophagy Inducer and a Mitochondrial Biogenesis Promoter

In a further embodiment of the invention, there is provided a composition comprising a combination of:
(a) a compound of formula (I), as described above, or a salt thereof, for example, Urolithin A;
(b) a further autophagy inducer, for example trehalose; and
(c) a mitochondrial biogenesis promoting agent, for example, a nicotinamide adenine dinucleotide precursor, for example, niacinamide.

In a further embodiment of the invention there is provided a composition, comprising:
(a) a compound of formula (I), or a salt thereof, as defined above;
(b) a further autophagy inducer; and
(c) a mitochondrial biogenesis promoting agent.

In a further embodiment of the invention, there is provided a composition comprising a combination of:
(a) a compound of formula (I), as described above, or a salt thereof, for example, Urolithin A;
(b) a further autophagy inducer, for example trehalose;
(c) a mitochondrial biogenesis promoting agent, for example, a nicotinamide adenine dinucleotide precursor, for example, niacinamide; and
(d) An anti-oxidant/promoter of blood circulation, for example, caffeine.

In a further embodiment of the invention, there is provided a composition comprising a combination of:
(a) a compound of formula (I), as described above, or a salt thereof, for example, Urolithin A,
   wherein the compound of formula (I) comprises 0.8% to 5% (w/w) of the composition;
(b) a further autophagy inducer, for example trehalose,
   wherein the autophagy inducer comprises 0.05% to 1% (w/w) of the composition;
(c) a mitochondrial biogenesis promoting agent, for example, a nicotinamide adenine dinucleotide precursor, for example, niacinamide,
   wherein the mitochondrial biogenesis promoting agent comprises 0.1% to 10% (w/w) of the composition.

In a further embodiment of the invention, there is provided a composition comprising a combination of:
(a) a compound of formula (I), as described above, or a salt thereof, for example, Urolithin A, wherein the compound of formula (I) comprises 0.8% to 5% (w/w) of the composition;
(b) a further autophagy inducer, for example trehalose, wherein the autophagy inducer comprises 0.05% to 1% (w/w) of the composition;
(c) a mitochondrial biogenesis promoting agent, for example, a nicotinamide adenine dinucleotide precursor, for example, niacinamide,
   wherein the mitochondrial biogenesis promoting agent comprises 0.1% to 10% (w/w) of the composition; and
(d) An anti-oxidant/promoter of blood circulation, for example, caffeine,
   wherein the anti-oxidant/promoter of blood circulation, for example, caffeine comprises 0.1% to 5% (w/w) of the composition.

Examples of autophagy inducers include, but not limited to, carbamazepine, clonidine, lithium, metformin, rapamycin (and rapalogs), rilmenidine, sodium valproate, verapamil, trifluoperazine, statins, tyrosine kinase inhibitors (for example, Akt-mTOR signaling inhibitors and beclin 1 tyrosine phosphorylation inhibitors), BH3 mimetics, caffeine, omega-3 polyunsaturated fatty acids, resveratrol, spermidine, vitamin D pterostilbene, fistein, genistein, quercetin, apigenin, kaempferol, minoxidil, actinonin, kinetin triphosphate, pifithrin-a, deferiprone, 1,10′-phenanthroline and trehalose, for example trehalose.

Examples of mitochondrial biogenesis promoting agents include, but are not limited to, PPAR-PGC-1α axis activators (for example, bezafibrate), AMPK activators (for example, resveratrol), Sirt1 agonists (for example, quercetin, resveratrol), anti-oxidants (such as L-carnitine, coenzyme Q₁₀, MitoQ₁₀ and other mitochondria-targeted antioxidants, N-acetylcysteine (NAC), vitamin C, vitamin E vitamin K1, vitamin B, sodium pyruvate and α-lipoic acid) and NAD precursors (for example, niacinamide).

Compounds of Formula (I) are members of the urolithin family; in particular, the compound of Formula (I) may be Urolithin A, Urolithin B, Urolithin C or Urolithin D. In one embodiment the compound of formula (I) is Urolithin A.

In a further embodiment, compositions of the invention comprise a compound of Formula (1) in the following amounts: about 0.8% to about 5% (w/w), about 0.8% to about 4% (w/w), about 0.8% to about 3% (w/w), about 0.8% to about 2% (w/w), about 0.8% to about 1% (w/w), or about 1% (w/w).

In a further embodiment, compositions of the invention comprise an autophagy inhibitor in the following amounts: about 0.05% to about 1%, about 0.1% to about 0.9%, about 0.1% to about 0.8%, about 0.1% to about 0.7%, about 0.1% to about 0.5%, about 0.1% to about 0.4%, about 0.1% to about 0.35%, about 0.2% to about 0.3%, or about 0.25%.

In a further embodiment, compositions of the invention comprise a mitochondrial biogenesis promoting agents in the following amounts: about 0.1% to about 10% (w/w), about 0.25% to about 9% (w/w), about 0.25% to about 8% (w/w), about 0.5% to about 7% (w/w), about 0.5% to about 6% (w/w), about 0.5% to about 5% (w/w), about 0.5% to about 4% (w/w), about 1% to about 4% (w/w), about 1% to about 3% (w/w), about 1% (w/w), about 2% (w/w), about 3% (w/w), about 4% (w/w) or about 5% (w/w).

In a further embodiment, compositions of the invention comprise a central nervous system stimulant, for example, caffeine, in the following amounts: about 0.1% to about 5% (w/w), about 0.1% to about 4% (w/w), about 0.1% to about 4% (w/w), about 0.1% to about 4% (w/w), about 0.1% to about 3% (w/w), about 0.1% to about 2% (w/w), about 0.1% to about 1% (w/w), about 0.2% to about 0.9% (w/w), about 0.3% to about 0.8% (w/w), about 0.3% to about 0.7% (w/w), about 0.3% to about 0.6% (w/w), about 0.4% to about 0.6% (w/w) or about 0.5% (w/w).

Compositions of the invention are suitable for both cosmetic and medical uses.

In a further embodiment, there is provided a composition of the invention comprising a compound of formula (I), a further autophagy inducer and a mitochondrial biogenesis promoting agent, for example, a cream or lotion, comprising about 0.8 to about 2.0% of a compound of formula (I), for example, urolithin A.

In a further embodiment, there is provided a composition of the invention comprising a compound of formula (I), a further autophagy inducer and a mitochondrial biogenesis promoting agent, for example, a cream or lotion, comprising about 0.8 to about 1.5% of a compound of formula (I), for example, urolithin A.

In a further embodiment, there is provided a composition of the invention comprising a compound of formula (I) a further autophagy inducer and a mitochondrial biogenesis promoting agent, for example, a cream or lotion, comprising about 0.8 to about 1.2% of a compound of formula (I), for example, urolithin A.

In a further embodiment, there is provided a composition of the invention comprising a compound of formula (I) a further autophagy inducer and a mitochondrial biogenesis promoting agent, for example, a cream or lotion, comprising about 1% of a compound of formula (I), for example, urolithin A.

In a further embodiment, there is provided a composition of the invention comprising a compound of formula (I), a further autophagy inducer and a mitochondrial biogenesis promoting agent, for example, a cream or lotion, for use in the treatment of one or more indications, described herein.

In a further embodiment, there is provided a composition of the invention comprising a compound of formula (I), a further autophagy inducer and a mitochondrial biogenesis promoting agent, for example, a cream or lotion, for use in the treatment of one or more indications, selected from:
(a) acne;
(b) contact dermatitis
(c) eczema;
(d) psoriasis;
(e) rosacea,
(f) lupus erythematosus;
(g) warts;
(h) shingles;
(i) hives;
(j) melasma;
(k) liver spots;
(l) lentigo;
(m) skin irritation;
(n) skin infection;
(o) skin inflammation;
(p) seborrheic dermatitis;
(q) Skin fungal infections (for example, candidiasis, athletes' feet);
(r) Skin cancer; and
(s) diaper rash.

In a further embodiment, there is provided a composition of the invention, wherein the composition is injectable and further comprises collagen and/or hyaluronic acid.

In a further embodiment, there is provided a composition of the invention, for example, a cream or lotion, comprising a compound of formula (I), for example, urolithin A, a further autophagy inducer, for example, trehalose and a mitochondrial biogenesis promoting agent, for example, an NAD precursors, for use in increasing anti-wrinkle efficiency For the avoidance of doubt, compositions of the invention may comprise one or more autophagy inducers, and one or more mitochondrial biogenesis promoting agents.

In a further embodiment there is provided a method of cosmetically treating skin using a composition of the invention comprising a compound of formula (I), a further autophagy inducer and a mitochondrial biogenesis promoting agent.

A method of cosmetic treatment of skin using an effective amount of a cosmetic composition comprising a compound of formula (I), a further autophagy inducer and a mitochondrial biogenesis promoting agent.

Use of a compound of formula (I), a further autophagy inducer and a mitochondrial biogenesis promoting agent as a cosmetic product.

Non-therapeutic use of cosmetic preparations comprising a compound of formula (I), a further autophagy inducer and a mitochondrial biogenesis promoting agent.

### 4) Uses of Compounds of Formula (I)

In a further embodiment of the invention, there is provided a composition of the invention for example, a composition comprising urolithin A, for one or more of the following uses:
(a) improving or maintaining skin barrier function;
(b) for limiting transepidermal water loss;
(c) for improving skin elasticity and firmness;
(d) for increasing or maintaining skin layer thickness;
(e) for increasing or maintaining the undulation index of dermal epidermal junctions in skin;
(f) for increasing or maintaining skin hydration;
(g) for reducing skin biological aging;
(h) for diminishing fine lines and wrinkles;
(i) for reduction of skin damage by irradiation, for example UV irradiation-induced skin damage.
(j) for protecting skin from damage caused by radiation, e.g., UV, beta or gamma radiation including during medical treatment for a condition such as a cancer.
(k) for reduction of hair damage by irradiation, for example UV irradiation-induced skin damage;
(l) for protecting hair from damage caused by radiation, e.g., UV, beta or gamma radiation including during medical treatment for a condition such as a cancer;
(m) for improving collagen organization in the skin;
(n) for improving skin matrix;
(o) improving skin physical properties;
(p) decreasing sun-induced skin redness;
(q) /for protection from sun-induced photoaging;
(r) Treating or preventing skin infection, and/or;
(s) Treating or preventing skin cancer.

In a further embodiment of the invention, there is provided a composition of the invention, for example, a composition comprising urolithin A, for one or more of the following uses:
(a) renewal and/or revitalization of skin appearance;
(b) enhancement of skin energy supply;
(c) improvement in skin texture;
(d) enhancement of skin radiance;
(e) skin rejuvenation;
(f) reduction of pore and micro line visibility;
(g) improvement in skin respiration.
(h) support optimal cellular function in skin;
(i) promote/activate skin detoxifying processes;
(j) promote collagen repair, and/or
(k) improve skin stem cell health.

In a further embodiment of the invention, there is provided a composition of the invention, for example, a composition comprising urolithin A, for one or more of the following uses:
(a) renewal and/or revitalization of skin appearance;
(b) enhancement of skin energy supply;
(c) improvement in skin texture;
(d) enhancement of skin radiance;
(e) skin rejuvenation/regeneration;
(f) reduction of pore and micro line visibility;
(g) improvement in skin respiration.
(h) support optimal cellular function in skin;
(i) promote/activate skin detoxifying processes;
(j) promote collagen repair, and/or
(k) improve skin stem cell health.

In a further embodiment of the invention, there is provided a composition of the invention, for example, a composition comprising urolithin A, for one or more of the following uses:
a) for maintaining skin barrier function;
b) for limiting transepidermal water loss;
c) for improving skin elasticity and firmness;
d) for increasing or maintaining skin layer thickness;
e) for increasing or maintaining skin hydration;
f) for improving anti-wrinkle efficiency;
g) for improving collagen organization in the skin;
h) for boosting collagen production
i) for favorizing plumper and youthful skin
j) for reducing skin biological aging
k) for improving skin health;
l) decreasing skin puffiness and dark circles under the eyes and/or
m) improving skin microarchitecture;

In a further embodiment of the invention, there is provided a composition of the invention, for example, a composition comprising urolithin A, for one or more of the following uses:
a) improving skin healthspan; and/or
b) improving skin longevity;

In a further embodiment of the invention, there is provided a composition of the invention for example, a composition comprising urolithin A, for one or more of the following uses:
(a) maintaining or enhancing hair thickness;
(b) hair follicle cell regeneration;
(c) hair follicle cell survival;
(d) hair stem cell growth and/or regeneration;
(e) hair cell survival;
(f) hair loss prevention,
(g) hair maintenance;
(h) scalp health improvement,
(i) improving or maintaining hair strength, and/or
(j) hair growth restoration promotion.

In a further embodiment of the invention, there is provided a composition of the invention for example, a composition comprising urolithin A, for one or more of the following uses:
a) maintaining or enhancing hair thickness;
b) hair follicle cell regeneration;
c) hair follicle cell survival;
d) hair stem cell growth and/or regeneration;
e) hair cell survival;
f) hair loss prevention,
g) scalp health improvement, and/or
h) hair growth restoration promotion.

In a further embodiment there is provided a method for:
a) maintaining or enhancing hair thickness;
b) hair follicle cell regeneration;
c) hair follicle cell survival;
d) hair stem cell growth and/or regeneration;
e) hair cell survival;
f) hair loss prevention,
g) promoting new hair growth;
h) scalp health improvement,
i) for improving survival of hair transplants; and/or
j) hair growth restoration promotion.

In a further embodiment of the invention, there is provided a composition of the invention for example, a composition comprising urolithin A, for one or more of the following uses:
a) enhancing hair stem cell function;
b) enhancing hair follicle elongation,
c) enhancing hair matrix proliferation, for example, as measured by Ki67 levels;
d) enhancing hair growing phase, (enhancing the duration of the anaphase);
e) hair matrix proliferation,
f) inhibition of hair matrix apoptosis; and
g) reducing melanin clumping, for example, in pigmentary units, for example, in hair follicle pigmentary units.

In a further embodiment of the invention, there are provided methods of reducing the biological aging of skin. Therefore, there is provided a composition of the invention for reducing skin biological age by greater than or equal to 5 years, for example, greater than equal to 2 years, for example greater or equal to 3 years, for example, greater than or equal to 4 years, for example, greater than or equal to 8 years, or, for example, greater than or equal to 10 years.

In a further embodiment, there is provided use of a composition of the invention comprising a compound of formula (I), for example, a cream or lotion, comprising about 0.8 to about 2.0% of a compound of formula (I), for example, urolithin A, wherein the use is one or more of the uses described herein.

In a further embodiment, there is provided use of a composition of the invention comprising a compound of formula (I), for example, a cream or lotion, comprising about 0.8 to about 1.5% of a compound of formula (I), for example, urolithin A, wherein the use is one or more of the uses described herein.

In a further embodiment, there is provided use of a composition of the invention comprising a compound of formula (I), for example, a cream or lotion, comprising about 0.8 to about 1.2% of a compound of formula (I), for example, urolithin A, wherein the use is one or more of the uses described herein.

In a further embodiment, there is provided use of a composition of the invention comprising a compound of formula (I), for example, a cream or lotion, comprising about 1% of a compound of formula (I), for example, urolithin A, wherein the use is one or more of the uses described herein.

In a further embodiment, there is provided use of a composition of the invention comprising a compound of formula (I), for example, a cream or lotion, comprising about 0.8 to about 2.0% of a compound of formula (I), for example, urolithin A, wherein the use is reducing intrinsic and/or extrinsic aging.

In a further embodiment, there is provided use of a composition of the invention comprising a compound of formula (I), for example, a cream or lotion, comprising about 0.8 to about 1.5% of a compound of formula (I), for example, urolithin A, wherein the use is reducing intrinsic and/or extrinsic aging.

In a further embodiment, there is provided use of a composition of the invention comprising a compound of formula (I), for example, a cream or lotion, comprising about 0.8 to about 1.2% of a compound of formula (I), for example, urolithin A, wherein the use is reducing intrinsic and/or extrinsic aging.

In a further embodiment, there is provided use of a composition of the invention comprising a compound of formula (I), for example, a cream or lotion, comprising about 1% of a compound of formula (I), for example, urolithin A, wherein the use is reducing intrinsic and/or extrinsic aging.

In a further embodiment, there is provided use of a composition of the invention comprising a compound of formula (I), for example, a cream or lotion, comprising about 0.8 to about 2.0% of a compound of formula (I), for example, urolithin A, wherein the use is reducing photoaging.

In a further embodiment, there is provided use of a composition of the invention comprising a compound of formula (I), for example, a cream or lotion, comprising about 0.8 to about 1.5% of a compound of formula (I), for example, urolithin A, wherein the use is reducing photoaging.

In a further embodiment, there is provided use a composition of the invention comprising a compound of formula (I), for example, a cream or lotion, comprising about 0.8 to about 1.2% of a compound of formula (I), for example, urolithin A, wherein the use is reducing photoaging.

In a further embodiment, there is provided use of a composition of the invention comprising a compound of formula (I), for example, a cream or lotion, comprising about 1% of a compound of formula (I), for example, urolithin A, wherein the use is reducing photoaging.

In a further embodiment, there is provided use of a composition of the invention comprising a compound of formula (I), for example, a cream or lotion, comprising about 0.8 to about 2.0% of a compound of formula (I), for example, urolithin A, wherein the use is reducing skin inflammation cause by ultra violet light exposure, for example ultra violet B light (UVB).

In a further embodiment, there is provided use of a composition of the invention comprising a compound of formula (I), for example, a cream or lotion, comprising about 0.8 to about 1.5% of a compound of formula (I), for example, urolithin A, wherein the use is reducing skin inflammation cause by ultra violet light exposure, for example ultra violet B light (UVB).

In a further embodiment, there is provided use a composition of the invention comprising a compound of formula (I), for example, a cream or lotion, comprising about 0.8 to about 1.2% of a compound of formula (I), for example, urolithin A, wherein the use is reducing skin inflammation cause by ultra violet light exposure, for example ultra violet B light (UVB).

In a further embodiment, there is provided use of a composition of the invention comprising a compound of formula (I), for example, a cream or lotion, comprising about 1% of a compound of formula (I), for example, urolithin A, wherein the use is reducing skin inflammation cause by ultra violet light exposure, for example ultra violet B light (UVB).

In a further embodiment, there is provided use of a composition of the invention comprising a compound of formula (I), for example, a cream or lotion, comprising about 0.8 to about 2.0% of a compound of formula (I), for example, urolithin A, wherein the use is reducing oxidative stress in skin.

In a further embodiment, there is provided use of a composition of the invention comprising a compound of formula (I), for example, a cream or lotion, comprising about 0.8 to about 1.5% of a compound of formula (I), for example, urolithin A, wherein the use is reducing oxidative stress in skin.

In a further embodiment, there is provided use a composition of the invention comprising a compound of formula (I), for example, a cream or lotion, comprising about 0.8 to about 1.2% of a compound of formula (I), for example, urolithin A, wherein the use is reducing oxidative stress in skin.

In a further embodiment, there is provided use of a composition of the invention comprising a compound of formula (I), for example, a cream or lotion, comprising about 1% of a compound of formula (I), for example, urolithin A, wherein the use is reducing oxidative stress in skin.

In a further embodiment, there is provided use of a composition of the invention comprising a compound of formula (I), for example, a cream or lotion, comprising about 0.8 to about 2.0% of a compound of formula (I), for example, urolithin A, wherein the use is improving skin health.

In a further embodiment, there is provided use of a composition of the invention comprising a compound of formula (I), for example, a cream or lotion, comprising about 0.8 to about 1.5% of a compound of formula (I), for example, urolithin A, wherein the use is improving skin health.

In a further embodiment, there is provided use a composition of the invention comprising a compound of formula (I), for example, a cream or lotion, comprising about 0.8 to about 1.2% of a compound of formula (I), for example, urolithin A, wherein the use is improving skin health.

In a further embodiment, there is provided use of a composition of the invention comprising a compound of formula (I), for example, a cream or lotion, comprising about 1% of a compound of formula (I), for example, urolithin A, wherein the use is improving skin health.

The invention further provides a novel use for improving skin barrier function.

In one embodiment, the invention provides a compound of formula (I), or a salt thereof, for example, urolithin A, for use in improving or maintaining skin barrier function.

In a further embodiment, the invention provides a compound of formula (I), or a salt thereof, for example, urolithin A, for use in the manufacture of a medicament for improving or maintaining skin barrier function.

In a further embodiment there is provided a method of improving or maintaining skin barrier function comprising administering an effective amount of a compound of formula (I), or a salt thereof, for example, urolithin A.

In one embodiment, the invention provides a compound of formula (I), or a salt thereof, for example, urolithin A, for use in the treatment of a disease or condition linked to a disrupted skin barrier.

In a further embodiment, the invention provides a compound of formula (I), or a salt thereof, for example, urolithin A, for use in the manufacture of a medicament for the treatment of a disease or condition linked to a disrupted skin barrier.

In a further embodiment there is provided a method for use in the treatment of a disease or condition linked to a disrupted skin barrier comprising administering an effective amount of a compound of formula (I), or a salt thereof, for example, urolithin A.

Examples of diseases or conditions linked to a disrupted skin barrier include, but limited to: atopic eczema, atopic dermatitis, acne, aged skin, dry skin, skin side effects of certain chronic medication and pruritus. For example, atopic dermatitis, acne and aged skin.

In one embodiment, the invention provides a compound of formula (I), or a salt thereof, for example, urolithin A, for use in increasing or maintaining skin hydration.

In a further embodiment, the invention provides a compound of formula (I), or a salt thereof, for example, urolithin A, for use in the manufacture of a medicament for increasing or maintaining skin hydration.

In a further embodiment there is provided a method for use in increasing or maintaining skin hydration comprising administering an effective amount of a compound of formula (I), or a salt thereof, for example, urolithin A.

In one embodiment, the invention provides a compound of formula (I), or a salt thereof, for example, urolithin A, for use in preventing and/or treating transepidermal water loss (TEWL).

In a further embodiment, the invention provides a compound of formula (I), or a salt thereof, for example, urolithin A, for use in the manufacture of a medicament for preventing and/or treating transepidermal water loss.

In a further embodiment there is provided a method of preventing and/or treating transepidermal water loss comprising administering an effective amount of a compound of formula (I), or a salt thereof, for example, urolithin A.

In one embodiment, the invention provides a compound of formula (I), or a salt thereof, for example, urolithin A, for use for improving skin elasticity and/or firmness.

In a further embodiment, the invention provides a compound of formula (I), or a salt thereof, for example, urolithin A, for use in the manufacture of a medicament for improving skin elasticity and/or firmness.

In a further embodiment, the invention provides a compound of formula (I), or a salt thereof, for example, urolithin A, for use in the manufacture of a cosmetic for improving skin elasticity and/or firmness.

In a further embodiment, there is provided a composition of the invention comprising a compound of formula (I), for example, urolithin A, for use as a cosmetic for one or more of the uses described herein.

In a further embodiment there is provided a method of cosmetically treating skin using a composition of the invention comprising a compound of formula (I) for use in one or more of the uses described herein.

A method of cosmetic treatment of skin using an effective amount of a cosmetic composition comprising a compound of formula (I), for use in one or more of the uses described herein.

Use of a compound of formula (I) as a cosmetic product, for one or more of the uses described herein.

Non-therapeutic use of cosmetic preparations comprising a compound of formula (I), for use in one or more of the uses described herein.

In a further embodiment there is provided a method of improving skin elasticity and/or firmness comprising administering an effective amount of a compound of formula (I), or a salt thereof, for example, urolithin A.

In one embodiment, the invention provides a compound of formula (I), or a salt thereof, for example, urolithin A, for use for increasing or maintaining skin layer thickness.

In a further embodiment, the invention provides a compound of formula (I), or a salt thereof, for example, urolithin A, for use in the manufacture of a medicament for increasing or maintaining skin layer thickness.

In a further embodiment there is provided a method of increasing or maintaining skin layer thickness comprising administering an effective amount of a compound of formula (I), or a salt thereof, for example, urolithin A.

In one embodiment, the invention provides a compound of formula (I), or a salt thereof, for example, urolithin A, for use for increasing or maintaining the undulation index of dermal epidermal junctions in skin.

In a further embodiment, the invention provides a compound of formula (I), or a salt thereof, for example, urolithin A, for use in the manufacture of a medicament for increasing or maintaining the undulation index of dermal epidermal junctions in skin.

In a further embodiment, the invention provides a compound of formula (I), or a salt thereof, for example, urolithin A, for use in the manufacture of a cosmetic for increasing or maintaining the undulation index of dermal epidermal junctions in skin.

In a further embodiment there is provided a method of increasing or maintaining the undulation index of dermal epidermal junctions comprising administering an effective amount of a compound of formula (I), or a salt thereof, for example, urolithin A, in skin.

In another embodiment of the invention, there is provided a compound of formula (I), or a salt thereof, as defined above, for decreasing winkle depth, particularly wrinkle depth of facial wrinkles, for example, wherein the decrease in wrinkle depth is at least about 15% below baseline. In a further embodiment, the wrinkle depth is at least 3.5%, at least 4%, at least 5% or at least 6%, at least 7%, at least 8%, at least 9%, at least 10%, at least 11%, at least 12%, at least 13%, at least 14%, or at least 15%. For example, after a period of about 2 weeks, 3 weeks, 4 weeks, 5 weeks, 6 weeks, 7 weeks or 8 weeks.

In another embodiment of the invention, there is provided a compound of formula (I), or a salt thereof, as defined above, for decreasing conspicuous area of wrinkles, particularly conspicuous area of facial wrinkles, for example, wherein the decrease in conspicuous area is at least about 4% below baseline. In a further embodiment, the conspicuous area is at least 5%, at least 6% or at least 7%. For example, after a period of about 2 weeks, 3 weeks, 4 weeks, 5 weeks, 6 weeks, 7 weeks or 8 weeks.

In another embodiment of the invention, there is provided a compound of formula (I), or a salt thereof, as defined above, for decreasing conspicuous length of wrinkles, particularly conspicuous length of facial wrinkles, for example, wherein the decrease in conspicuous length is at least about 6% below baseline. In a further embodiment, the conspicuous length is at least 7%, at least 8%, at least 9%, or at least 10%. For example, after a period of about 2 weeks, 3 weeks, 4 weeks, 5 weeks, 6 weeks, 7 weeks or 8 weeks.

In another embodiment of the invention, there is provided a compound of formula (I), or a salt thereof, as defined above, for decreasing conspicuous depth of wrinkles, particularly conspicuous depth of facial wrinkles, for example, wherein the decrease in conspicuous depth is at least about 3.5% below baseline. In a further embodiment conspicuous depth is at least 4%, at least 5% or at least 6%. For example, after a period of about 2 weeks, 3 week, 4 weeks, 5 weeks, 6 weeks, 7 weeks or 8 weeks.

In another embodiment of the invention, there is provided a compound of formula (I), or a salt thereof, as defined above, for decreasing conspicuous volume of wrinkles, particularly conspicuous volume of facial wrinkles, for example, wherein the decrease in conspicuous volume is at least about 4% below baseline. In a further embodiment conspicuous volume is at least 5%, at least 6% or at least 7%. For example, after a period of about 2 weeks, 3 weeks, 4 weeks, 5 weeks, 6 weeks, 7 weeks or 8 weeks.

In another embodiment of the invention, there is provided a compound of formula (I), or a salt thereof, as defined above, for decreasing one of the following wrinkle parameter, particularly one of more of the following facial wrinkle parameters
(a) conspicuous area;
(b) conspicuous length;
(c) conspicuous depth; and/or
(d) conspicuous volume.
   For example, with a percentage below baseline and a period of time as recited for each integer above.

In a further embodiment, there is provided a composition of the invention comprising a compound of formula (I), for example, urolithin A, for use as a cosmetic for decreasing one of the following facial wrinkle parameter,
(a) conspicuous area;
(b) conspicuous length;
(c) conspicuous depth; and/or
(d) conspicuous volume.

For example, with a percentage below baseline and a period of time as recited for each integer above.

In a further embodiment there is provided a method of cosmetically treating facial wrinkles using a composition of the invention comprising a compound of formula (I) for decreasing one of the following facial wrinkle parameter,
(a) conspicuous area;
(b) conspicuous length;
(c) conspicuous depth; and/or
(d) conspicuous volume.
   For example, with a percentage below baseline and a period of time as recited for each integer above.

A method of cosmetic treatment of facial wrinkles using an effective amount of a cosmetic composition comprising a compound of formula (I), for decreasing one of the following facial wrinkle parameter,
(a) conspicuous area;
(b) conspicuous length;
(c) conspicuous depth; and/or
(d) conspicuous volume.

For example, with a percentage below baseline and a period of time as recited for each integer above. In one embodiment, the invention provides a compound of formula (I), or a salt thereof, for example, urolithin A, for administration to head, scalp or hair.

In a further embodiment, the invention provides a compound of formula (I), or a salt thereof, for example, urolithin A, for the manufacture or a medicament for administration to head, scalp or hair, for example, for use in any one of the uses described herein.

In a further embodiment there is provided a method comprising administering an effective amount of a compound of formula (I), or a salt thereof, for example, urolithin A, to head, scalp and hair, for example, for use in any one of the uses described herein.

In a further embodiment there is provided a cosmetic method comprising administering an effective amount of a compound of formula (I), or a salt thereof, for example, urolithin A, to head, scalp and hair, for example, for use in any one of the uses described herein.

Non-therapeutic use of cosmetic preparations, for administration to head, scalp or hair, comprising a compound of formula (I), for use in one or more of the uses described herein.

Use of a compound of formula (I) as a cosmetic product, for administration to head, scalp or hair, for one or more of the uses described herein.

In one embodiment, the invention provides a compound of formula (I), or a salt thereof, for example, urolithin A, for use, for example a cosmetic use, for
(a) maintaining or enhancing hair thickness;
(b) hair follicle cell regeneration;
(c) hair follicle cell survival;
(d) hair stem cell growth and/or regeneration;
(e) hair cell survival;
(f) hair loss prevention,
(g) hair maintenance, Hair strength preservation or maintenance,
(h) scalp health improvement, and/or
(i) hair growth restoration promotion.

In a further embodiment, the invention provides a compound of formula (I), or a salt thereof, for example, urolithin A, for use in the manufacture or a medicament, or for use in the manufacture of a cosmetic product, for
a) maintaining or enhancing hair thickness;
b) hair follicle cell regeneration;
c) hair follicle cell survival;
d) hair stem cell growth and/or regeneration;
e) hair cell survival;
f) hair loss prevention,
g) scalp health improvement, and/or
h) hair growth restoration promotion

In a further embodiment there is provided a method, for example, a cosmetic method, for:
a) maintaining or enhancing hair thickness;
b) hair follicle cell regeneration;
c) hair follicle cell survival;
d) hair stem cell growth and/or regeneration;
e) hair cell survival;
f) hair loss prevention,
g) scalp health improvement, and/or
h) hair growth restoration promotion;
   comprising administering an effective amount of a compound of formula (I), or a salt thereof, for example, urolithin A.

In one embodiment, the administration, for uses of the invention, is via topical administration of a compound of formula (I), or a salt thereof, for example, urolithin A. In a further embodiment the administration, for uses of the invention, is via injection of a compound of formula (I), for example, urolithin A.

International patent applications WO 2018/162650 (Amazentis) and WO 2018/162651 (Amazentis) disclose optimised dose ranges for oral administration of urolithins. The present application includes topical administration of a compound of formula (I), for example Urolithin A, which requires a different optimised dose range to that required for oral administration. Therefore, the present invention also provides an optimised dose range for administration of a compound of formula (I) by topical administration to the skin. Therefore, there is provided a compound of formula (I), or a salt thereof,

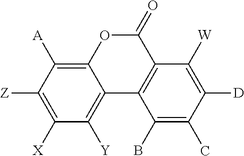

wherein:
A, B, C and D are each independently selected from H and OH;
W, X and Y are each independently selected from H and OH; and
Z is selected from H and OH,
for topical administration at a concentration in the range 0.1 to 5% w/w.

In another embodiment the concentration range of a compound of formula (I), or a salt thereof, is 0.8% to 4% of the composition, for example, 0.8% to 3% of the composition, for example, 0.8% to 2% w/w of the composition, for example 1% to 2% w/w of the composition, for example 1% to 1.5% w/w, for example about 1% w/w, such as 0.8%, 0.9%, 1%, 1.5% or 2% w/w of the composition. In a further embodiment of the invention, the concentration range of a compound of formula (I), or a salt thereof, is 1% to 5% w/w, for example, 2% to 5% w/w, for example, 3% to 5% w/w, such as 4% to 5% w/w.

In another embodiment of the invention, there is provide a compound of formula (I), or a salt thereof, as defined above, for topical administration at a concentration in the range 0.8% to 5% for cosmetic use.

In another embodiment of the invention, there is provided a compound of formula (I), or a salt thereof, as defined above, for topical administration at a concentration in the range 0.8% to 5% for the treatment and/or prophylaxis of a skin condition, skin disease or skin disorder in a subject.

In another embodiment of the invention, there is provided a compound of formula (I), or a salt thereof, as defined above, for topical administration at a concentration in the range 0.8% to 5% for one or more of the following uses:
(a) improving or maintaining skin barrier function;
(b) for preventing and/or treating transepidermal water loss;
(c) for improving skin elasticity and firmness;
(d) for increasing or maintaining skin layer thickness;
(e) for increasing or maintaining the undulation index of dermal epidermal junctions in skin;
(f) for increasing or maintaining skin hydration;
(g) for reducing skin biological aging;
(h) for diminishing fine lines and wrinkles;
(i) for reduction of skin damage by irradiation, for example UV irradiation-induced skin damage;
(j) for protecting skin from damage caused by radiation, e.g., UV, beta or gamma radiation including during medical treatment for a condition such as a cancer.
(k) for reduction of hair damage by irradiation, for example UV irradiation-induced skin damage; and/or
(l) for protecting hair from damage caused by radiation, e.g., UV, beta or gamma radiation including during medical treatment for a condition such as a cancer.
   (3) Compositions Comprising Micronized Compound of Formula (I) and their Uses

An important aspect of a topical skin formulation, especially a cosmetic topical skin formulation, is the sensory impact and user experience. In addition to developing novel skin formulations comprising urolithins, we have found that a particular particle size distribution gives an optimised sensory impact and user experience. Therefore, in a further embodiment of the invention there is provided a compound of formula (I), or salt thereof,

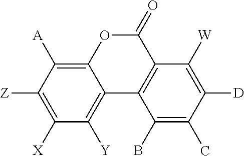

wherein:
A, B, C and D are each independently selected from H and OH,
W, X and Y are each independently selected from H and OH; and
Z is selected from H and OH,
and wherein the compound or salt has a D50 size in the range 1 to 10 μm and a D90 size in the range 4 to 25 μm.

In a further embodiment of the invention, there is provided a composition of the invention comprising a compound of formula (I) or salt thereof, as defined above, wherein the compound or salt has a D50 size in the range 1 to 10 μm and a D90 size in the range 4 to 25 μm.

In a further embodiment of the invention, there is provided a composition of the invention comprising a compound of formula (I) or salt thereof, as defined above, wherein the compound or salt has a D10 size greater than or equal to 0.1 μm, D50 size in the range 2 to 8 μm and a D90 size in the range 6 to 20 μm.

In a further embodiment of the invention, there is provided a composition of the invention comprising a compound of formula (I) or salt thereof, as defined above, wherein the compound or salt has a D50 size in the range 2 to 8 μm and a D90 size in the range 6 to 20 μm.

In a further embodiment of the invention, there is provided a composition of the invention comprising a compound of formula (I) or salt thereof, as defined above, wherein the compound or salt has a D10 size not less than 0.1 μm, D50 size not more than 15 μm and a D90 size not more than 25 μm.

In a further embodiment of the invention, there is provided a composition of the invention comprising a compound of formula (I) or salt thereof, as defined above, wherein the compound or salt has a D10 size in the range 0.1 to 2 μm, D50 size in the range 5 to 12 μm and a D90 size in the range 12 to 25 μm.

In a further embodiment of the invention, there is provided a composition of the invention comprising a compound of formula (I) or salt thereof, as defined above, wherein the compound or salt has a D50 size in the range 1 to 10 μm and a D90 size in the range 4 to 25 μm, wherein the composition is for topical administration at a concentration in the range 0.1% to 20%, 0.1% to 10%, 0.1% to 5% for cosmetic use, for example, about 0.1% to about 2%, about 0.125%, about 0.5%, about 1.0%, about 1.5%, about 2%, about 5%, about 10%, about 12%, about 15% or about 20%.

In another embodiment of the invention, there is provided a compound of formula (I), or a composition comprising a compound of formula (I), or a salt thereof, as defined above, wherein the compound or salt has a D₅₀ size in the range 1 to 10 μm and a D₉₀ size in the range 4 to 25 μm for one or more of the following uses:
(a) improving or maintaining skin barrier function;
(b) for preventing and/or treating transepidermal water loss;
(c) for improving skin elasticity and firmness;
(d) for increasing or maintaining skin layer thickness;
(e) for increasing or maintaining the undulation index of dermal epidermal junctions in skin;
(f) for increasing or maintaining skin hydration;
(g) for reducing skin biological aging;
(h) for diminishing fine lines and wrinkles;
(i) for reduction of skin damage by irradiation, for example UV irradiation-induced skin damage.
(j) for protecting skin from damage caused by radiation, e.g., UV, beta or gamma radiation including during medical treatment for a condition such as a cancer.
(k) for reduction of hair damage by irradiation, for example UV irradiation-induced skin damage;
(l) for protecting hair from damage caused by radiation, e.g., UV, beta or gamma radiation including during medical treatment for a condition such as a cancer
(m) for improving collagen organization in the skin;
(n) for improving skin matrix;
(o) improving skin physical properties;
(p) decreasing sun-induced skin redness; and/or
(q) for protection from sun-induced photoaging.

Urolithins are poorly soluble in aqueous media, leading to poor dispersion and agglomeration of urolithins, leading to poorly bioavailability in topical formulations. Therefore, in addition to compositions comprising a compound of formula (I) and trehalose and/or an NAD precursor, and optionally caffeine, the invention also provide topical compositions comprising a compound of formula (I) and good solubility of urolithins leading to high bioavailability. Therefore, according to a further embodiment of the invention, there is provided a topical composition, comprising:
(a) a compound of formula (I); or a salt thereof, and
(b) one or more of the following:
   i) an aqueous phase;
   ii) a water soluble emollient and humectant;
   iii) a co-solvent/wetting agent
   iv) an occlusive moisturizer and barrier repair ingredient, such as a vitamin rich occlusive moisturizer and barrier repair ingredient;
   v) a carrier oil/emollient, such as a C₈₋₁₄ medium chain mono-este;
   vi) an emulsifier;
   vii) a structuring agent;
   viii) a polymeric thickener and emulsifier; for example, a C15-19 alkane,
   ix) a polymeric anionic thickener; and/or
   x) a preservative.

According to a further embodiment of the invention, there is provided a topical composition, comprising:
(a) a compound of formula (I), or a salt thereof;
(b) a carrier oil/emollient, such as a C₈₋₁₄ medium chain mono-ester; and
(c) an emulsifier.

According to a further embodiment of the invention, there is provided a topical composition, comprising:
(a) a compound of formula (I), or a salt thereof;
(b) a water soluble emollient and humectant;
(b) a carrier oil/emollient, such as a C₈₋₁₄ medium chain mono-ester; and
(c) an emulsifier.

According to a further embodiment of the invention, there is provided a topical composition, comprising:
(a) a compound of formula (I), or a salt thereof;
(b) a water soluble emollient and humectant;
(c) a carrier oil/emollient, such as a C₈₋₁₄ medium chain mono-ester;
(d) an emulsifier
(e) a structuring agent; and
(f) a polymeric thickener.

Examples of water soluble emollient and humectants include: glycerine, butylene glycol, 1,2-hexanediol

Examples of co-solvent/wetting agents include: ethanol, glycols (such as butylene glycol), glycerin, ethoxydiglycol, dimethyl isosorbide.

Examples of occlusive moisturizer and barrier repair ingredients include butyrospermum parkii (shea) butter; Examples of carrier oil/emollients include: coco-caprylate/caprate, such as mono-, di-, and tri-esters of spanning the range of C1 to C22. Fats and oils (defined as triesters of glycerol), natural or synthetic. Lamellar forming structures such as those mentioned above, cholesterol, phytosterol.

Examples of emulsifiers include; sorbitan oleate, PEG-100 stearate,

Examples of structuring agents include; stearic acid

Examples of polymeric thickener and emulsifiers include: hydroxyethyl acrylate/sodium acryloyldimethyl taurate copolymer,

Examples of polymeric anionic thickeners include: carbomer

Examples of preservatives include: phenoxyethanol.

Examples of surfactants include C8 to C22 ionic or non-ionic. The more water soluble ones (higher Hydrophile Lipophile Balance or HLB) tend to disrupt the skin barrier and allow substances to transverse more readily.

Some surfactants can also form micelles or liposomes that more readily transverse the stratum corneum.

In a further aspect of the invention there is provided a method for the preparation of a topical composition of the invention, comprising:
(a) In a first vessel heat water, for example to about 80° C.;
(b) In a second vessel add oil, an emulsifier and a compound of formula (I);
(c) Heat the second vessel, for example to a temperature of about 80° C.;
(d) Add the contents of the second receptacle to the first receptable; and
(e) cool the resulting mixture, for example to about 40° C.

In a further embodiment of the invention there is provided a composition comprising one or more components as listed in Column A of Table 1 in a concentration range as listed in Column C of Table 1.

In a further embodiment of the invention there is provided a composition comprising one or more components as listed in Column B of Table 1 in a concentration range as listed in Column C of Table 1.

For the avoidance of doubt, components of the compositions defined in Table 1 add up to 100%.

TABLE 1

| | Column B | Column C |
|---|---|---|
| Column A | INCI Name (Mixed) | %(w/w) |
| Carrier fluid | Water | 60 to 80% |
| Water soluble emollient | GLYCERIN | 1 to 20% |
| and humectant. | | |
| Water soluble emollient | BUTYLENE GLYCOL | 1 to 5% |
| and humectant. Co- | | |
| solvent. Wetting agent. | | |
| Occlusive moisturizer and | BUTYROSPERMUM PARKII (SHEA) | 0.5 to 20% |
| barrier repair ingredient. | BUTTER | |
| Vitamin Rich. | | |
| Carrier oil. Emollient. A | COCO-CAPRYLATE/CAPRATE | 0.5 to 20% |
| medium chain mono- | | |
| ester. C8 to C14. | | |
| Lamellar emulsifier | GLYCERYL STEARATE | 1 to 10% |
| Emollient. Aesthetic | C₁₅₋₁₉ ALKANE | 1 to 5% |
| modifier. | | |
| Compound of formula (I) | Urolithin A | 0.1 to 2% |
| Water soluble emollient | 1,2-HEXANEDIOL | 0.1 to 2% |
| and humectant. Co- | | |
| solvent. Wetting agent. | | |
| Emulsifier (w/o tendency) | SORBITAN OLEATE | 0.1 to 5% |
| When neutrsalized, strong | STEARIC ACID | 0.1 to 10% |
| o/w emulsifier. Otherwise | | |
| a structuring agent. | | |
| Powerful o/w emulsifier. | PEG-100 STEARATE | 0.1 to 10% |
| Often used with Glyceryl | | |
| Stearate to form lamellar | | |
| emulsions. | | |
| Polymeric thickener and | HYDROXYETHYL | 0.05 to 2% |
| emulsifier. Often carried | ACRYLATE/SODIUM | |
| in C15-19 Alkane as a | ACRYLOYLDIMETHYL TAURATE | |
| raw material | COPOLYMER | |
| Polymeric anionic | CARBOMER | 0.05 to 2% |
| thickener. | | |
| Emollient and component | SQUALANE | 0.01 to 10% |
| of the skin's Natural | | |
| Moisturizing Factor. | | |
| Surface active | CAPRYLYL GLYCOL | 0.05 to 2% |
| preservative. Cell | | |
| disruptor. | | |
| Neutralizer pH adjuster | SODIUM HYDROXIDE | 0.01 to 0.2% |
| Solubilizer, o/w | POLYSORBATE 60 | 0.01 to 5% |
| emulsifier. Part of the | | |
| Hydroxyethyl | | |
| Acrylate/Sodium | | |
| Acryloyldimethyl Taurate | | |
| Copolymer, C15-19 | | |
| Alkane Polysorbate | | |
| ingredient system | | |
| (Simulgel Series from | | |
| Seppic) | | |
| Surface active | ETHYLHEXYLGLYCERIN | 0.01 to 2% |
| preservative. Cell | | |
| disruptor. | | |
| Antioxidant Vitamin | TOCOPHEROL | 0.01 to 5% |
| Likely incidental w/o | SORBITAN ISOSTEARATE | 0.001 to 0.05% |
| emulsifier | | |
| Likely incidental oil | HELIANTHUS ANNUUS | 0.001 to 0.05% |
| | (SUNFLOWER) SEED OIL | |

The invention provides topical compositions, comprising a compound of formula (I). However, there may be advantages to a binary administration regime for the delivery of a compound of formula (I) comprising topical administration of a composition comprising a compound of formula (1) combined with oral administration of a composition comprising a compound of formula (1). For example, administration of a topical composition with concentration of a compound of formula (I) of between 0.8% to 5% and an oral composition comprising 250 mg to 2000 mg of a compound of formula (I), for example, a topical composition with 0.8% to 2% w/w of a compound of formula (I) and an oral composition comprising 500 mg, 1000 mg, 1500 mg or 2000 mg of a compound of formula (I).

### BRIEF DESCRIPTION OF THE FIGURES

FIG. 1: Prostaglandin E2 (PGE2) levels measured via an ELISA kit in the supernatant of reconstituted human epidermis (RHE) at the indicated condition. Data are shown as PGE2 concentration expressed as pg/mL.

FIG. 2: Normalized Enrichment Scores (NES) of the gene set “collagen fibril organization” in a published skin aging study (Glass et al (2013) Genome Biol 14(7), R75) and in the study with application of a Mitopure 1% containing product.

FIG. 3: Bar plot representing several collagen genes that are downregulated with skin aging, indicated by negative aging beta value. Data analysed from the study: [Glass et al (2013) Genome Biology Article number: R75]

FIG. 4: Bar plot representing several collagen genes that are upregulated following application of Mitopure on skin for 8 weeks, calculated as change of expression comparing end of application to baseline, and values are normalized over the change in expression levels in the placebo control group.

FIG. 5: Heatmap on genes associated with improved skin health after Urolithin A treatment.

FIG. 6: Selected genes associated with improved mitophagy and reduced collagen degradation after Urolithin A treatment in human keratinocytes and dermal fibroblasts.

FIG. 7: Anti-wrinkle efficacy with Urolithin A alone cream compared to a cream containing Urolithin A, trehalose and niacinamide.

FIG. 8: Change in Skin Hydration with Urolithin A alone cream compared to a cream containing Urolithin A, trehalose and niacinamide

FIG. 9: Reduction in skin wrinkle depth after application of 1% urolithin A (Mitopure) after 2 weeks and 8 weeks.

FIG. 10: showing reduction at 1.6MED dose (moderate sunburn) with 1% Urolithin A

FIG. 11. CONSORT Diagram 55 Participants were screened in the placebo-controlled, first aging study of which n=48 middle aged women subject with mean age of 58.6±6.6 years were randomized to two study arms with a split-face/arm study design with a lower dose active group compared to placebo and a second higher dose active group compared to placebo. Skin biopsies were conducted on the volar forearm in adjacent areas at the start and end of the 8-week intervention. All participants completed the trial and were included in the final analysis.

FIG. 12. UA 1% topically applied in a basic skin cream (A) in the first aging study or in a more defined matrix of day (B) and night (C) skin cream formulations in the second aging study, exhibited significant anti-wrinkle effects in middle-aged subjects with moderate facial wrinkles after 8-weeks of topical application (p<0.05). There was no significant impact on skin barrier function (D). Skin hydration was improved upon topical application of different UA containing creams (E, F).

FIG. 13. Mitopure positively impacts biomarkers associated with improved skin health
(A) GSEA results from RNA-seq in skin biopsies showing the most significant Gene Ontology Biological Processes (GO-BP) pathways that are significantly enriched in Mitopure compared to vehicle. Terms with positive and negative normalized enrichment scores (NES), are categorized as “activated” and “suppressed”, respectively. Pathways are sorted by gene ratio, dot sizes scaled by the number of core enriched genes, and dot color reflects the statistical significance of the enrichment (Benjamini-Hochberg-adjusted p-value, P-adj).
(B) Bar plot showing increased expression in core genes of the “Collagen fibril organization” pathway in skin biopsies exposed to Mitopure compared to the vehicle group (top). Bar plot of Aging Beta value for same genes derived from the skin aging trial (bottom). Only common core enriched genes between Mitopure and skin aging dataset are shown.
(C-D) Expression of genes encoding for collagen degrading enzymes (MMP1, MMP3) and autophagy/mitophagy proteins (PINK1, PARK2, SQSTM1, ULK1) in human dermal fibroblasts (C) and human keratinocytes (D) treated for 24 hours with DMSO or the indicated doses of Mitopure.
(E) Expression of genes encoding for autophagy/mitophagy proteins (PINK1, PARK2, SQSTM1, ULK1) in human reconstituted epidermis after daily application for 72 hours of Vehicle or Mitopure 1% containing moisturizers. Data expressed as Fold change over Vehicle+/−sem. *p<0.05; **P<0.01; ***P<0.005; ***P<0.001 One-Way ANOVA.

FIG. 14: CONSORT Flow Diagram for Aging Study 2 and UVB Erythema Trial.

FIG. 15: Effect of urolithin A on wrinkles.

FIG. 16. Impact of Mitopure on biomarkers associated with improved skin health
(A) Heatmap of log 2 fold changes from core enriched genes of the “Collagen fibril organization” pathway comparing end of study to baseline, in Mitopure skin biopsies after normalization over vehicle controls. Only subjects present in both visits and both treatments are considered in visualization. Rows (genes) were hierarchically clustered with a euclidean distance metric using complete linkage.
(B) Normalized enrichment score (NES) of “Collagen fibril organization” pathway for Mitopure normalized over vehicle comparison and for the “aging” dataset. The significance level of the BP enrichment for each comparison/dataset is denoted accordingly.
(C) Expression of the gene encoding for Collagen Type I Alpha 1 Chain (COL1A1) in human dermal fibroblasts treated for 24 hours with DMSO or the indicated doses of Mitopure.
(D) Expression of genes encoding for collagen degrading enzymes (MMP1, MMP3) in human reconstituted epidermis after daily application for 72 hours of Vehicle or Mitopure 1% containing moisturizer. Data expressed as Fold change over Vehicle+/−sem.

FIG. 17: Anti-wrinkle effect of 1% urolithin A on wrinkles.

FIG. 18: Lack of anti-wrinkle effect of 0.5% urolithin A on wrinkles.

FIG. 19: Effect of Day Cream and Night Cream on wrinkles after 8 weeks

FIG. 20: Effect of Day Cream, Night Cream and serum on skin hydration after 8 weeks

FIG. 21: Table summarising effects of urolithin A cream on skin parameters.

FIG. 22: Effect of Day Cream and Night Cream on skin hydration after 8 weeks.

FIG. 23: Face and volar forearms (for biopsies).

### DETAILED DESCRIPTION

As described above, the invention provides a composition comprising trehalose, and a urolithin.

The trehalose typically makes up at least 0.1% w/w of the composition of the invention, for example, at least 0.2% w/w, at least 0.3% w/w, at least 0.4% w/w, for example, 0.5% w/w of the composition. For example, the trehalose makes up 0.1% to 20% w/w, for example 0.1% to 15% w/w, for example, 0.1% to 10% w/w, for example, 0.5% to 5% w/w, for example 0.1% to 5% w/w of the composition, for example 0.1% to 2% of the composition, for example, 0.1% to 1% of the composition, for example, 0.2% to 0.8% w/w of the composition, for example 0.3% to 0.7% w/w of the composition, such as 0.4% to 0.6% w/w of the composition.

Furthermore, the invention provides a composition comprising niacinamide and a urolithin or trehalose, niacinamide and a urolithin.

The niacinamide typically makes up at least 0.1% w/w of the composition of the invention, for example, at least 0.5% w/w, at least 1% w/w, at least 2% w/w, for example, 1%, 3% or 5% w/w of the composition. For example, the niacinamide makes up 0.1% to 10% w/w of the composition, for example 0.5% to 8% of the composition, for example, 0.5% to 7% of the composition, for example, 0.5% to 6% w/w of the composition, for example 0.5% to 5% w/w of the composition, for example, 1% to 3% of the composition, such as 1%, 2%, 3%, 4% or 5% w/w of the composition.

The compound of formula (I) typically makes at least 0.5% of the composition, for example, 0.5% w/w or 1% of the composition. For example, the compound of formula (I) makes up 0.5% to 2% w/w of the composition, such as 0.5% or 1% w/w of the composition.

The compound of formula (I) typically makes up at least 0.8% w/w of the composition of the invention, for example, at least 0.9% w/w, at least 1.0% w/w, at least 1.1% w/w, at least 1.2% w/w, at least 1.3% w/w, at least 1.4% w/w, at least 1.5%, at least 1.6%, at least 1.7%, at least 1.8%, at least 1.9% of the composition. For example, the compound of formula (I) makes up 0.8% to 2% w/w of the composition, for example, 0.1% to 1.8% w/w, for example, 0.8% to 1.5% w/w, for example 0.8% to 1.3%% of the composition, for example, 1.0% to 1.5% of the composition, for example, 1% to 1.3% w/w of the composition, such as 1% or 1.5% w/w of 2.0% of the composition.

The weight ratio (w/w) of the urolithin to trehalose is generally in the range 10:1 to 1:1, for example, 5:1 to 1:1, for example, 4:1 to 1:1.

The weight ratio (w/w) of urolithin to niacinamide is generally in the range 1:200 to 1:1, for example, 1:10 to 1:1, for example 1:5 to 1:1, for example 1:3 to 1:1, for example, 1:1.

The weight ratio of trehalose to niacinamide is generally in the range 1:20 to 1:1, for example 1:10 to 1:1, for example, 1:5 to 1:1, for example 1:4 to 1:1, for example, 1:5 to 1:1.

### Urolithins:

Urolithins are metabolites produced by the action of mammalian, including human, gut microbiota on ellagitannins and ellagic acid. Ellagitannins and ellagic acid are compounds commonly found in foods such as pomegranates, nuts and berries. Ellagitannins are minimally absorbed in the gut themselves. Urolithins are a class of compounds with the representative structure (I) shown above. The structures of some particularly common urolithins are described in Table 2 below, with reference to structure (I).

TABLE 2

| | Substituent of structure (I) | | | | | |
|---|---|---|---|---|---|---|
| | A | B | C | D | W, X and Y | Z |
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Urolithin E | OH | OH | H | OH | H | OH |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO2014/004902, WO 2015/100213 and WO 2019/168972.

Urolithins of any structure according to structure (I) may be used in compositions of the invention. Particularly suitable compounds for use in compositions of the invention are the naturally-occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B and C are all H, and D and Z are both OH, then the compound is Urolithin A. Preferably, the Urolithin used in a formulation of the invention is Urolithin A, Urolithin B, Urolithin C or Urolithin D. Most preferably, the Urolithin used in a formulation of the invention is Urolithin A.

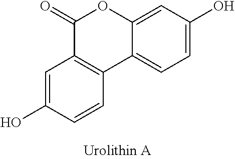 [SMILES: O=c1oc2cc(O)ccc2c2ccc(O)cc12]

In one embodiment, the urolithin for use in compositions of the invention is micronized. Micronization enables the urolithin to disperse in formulation or dissolve more rapidly. If micronized urolithin is used, then preferably, the urolithin has a D₅₀ size of under 8 μm—that is to say that 50% of the urolithin by mass has a particle diameter size of under 8 μm. More preferably, the urolithin has a D₅₀ size of under 7 μm, for example under 6 μm, for example under 5 μm, for example under 4 μm, for example under 2.5 μm. More preferably, the urolithin has a D₅₀ in the range 1.5 to 8.5 μm, for example 2 to 8 μm, for example 1.5 to 3.5 μm, for example 1.5 to 2.5 μm, for example 3 to 8 μm, for example 3 to 6 μm. Preferably, the urolithin has a D₉₀ size of under 25 μm. More preferably, the urolithin has a D₉₀ size of under 15 μm, for example under 12 μm. The urolithin preferably has a D₉₀ in the range 5 to 25 μm, for example 9 to 25 μm, for example 10 to 25 μm, for example 10 to 20 μm, for example 5 to 15 μm. Preferably, the urolithin has a D₁₀ in the range 0.5-3 μm, for example, 0.5 to 2.5 μm, for example, 0.5 to 2.0 μm, for example, 0.5 to 1.5 μm. Preferably, the urolithin has a D₉₀ in the range 5 to 25 μm, a D₅₀ in the range 1.5 to 8.5 μm and a D₁₀ in the range 0.5 to 3 μm.

Micronisation can be achieved by methods established in the art, for example compressive force milling, hamermilling, universal or pin milling, or jet milling (for example spiral jet milling or fluidised-bed jet milling) may be used. Jet milling is especially suitable.

### Forms of Compositions:

Compositions of the invention are provided in a variety of forms, for example, forms suitable for topical administration. Such compositions for topical administration include make-up, lotions, creams, shampoos, serums, sprays, gels, masks, toners, micellar waters, soaps, balms, mousses, oils, sunscreens, milks, exfoliators, hair treatments, lotions, and cleansers.

In one embodiment of the invention, there is provided a composition of the invention in combination with a pharmaceutically or cosmetically acceptable carrier.

‘Pharmaceutically acceptable carrier’ or ‘cosmetically-acceptable carrier’ means any carrier, diluent or excipient which is compatible with the other ingredients of the formulation and not deleterious to the recipient. The active agent may be formulated into dosage forms according to standard practices in the field of pharmaceutical preparations. See Alphonso Gennaro, ed., Remington's Pharmaceutical Sciences, 18th Edition (1990), Mack Publishing Co., Easton, Pa.

For topical administration, compositions of the invention may be mixed with a suitable carrier or diluent such as water, an aqueous buffer, an oil (particularly a vegetable oil), ethanol, saline solution, aqueous dextrose (glucose) and related sugar solutions, glycerol, or a glycol such as propylene glycol or polyethylene glycol. Solutions for topical administration preferably contain a water-soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for topical administration may take the form of an aqueous or non-aqueous solution, dispersion, suspension or emulsion.

Topical formulations may also be combined with at least one excipient such as fillers, binders, humectants, disintegrating agents, solution retarders, absorption accelerators, wetting agents, absorbents or lubricating agents.

Topical compositions comprising low dose formulations of the invention may further comprise ceramides, which are well known in the art. See, for example, Meckfessel and Brandt, “The Structure Function and Importance of Ceramides in Skin and Their Use as Therapeutic Agents in Skin-Care Products,” J Am Acad Dermatol 2014; 71:177-84 (herein incorporated by reference in its entirety.) In further preferred embodiments, the topical formulation is used several times, giving doses over a period of time, e.g., a daily dose or twice daily treatment for a week or more.

Examples of ceramides that can be formulated as described herein include, but are not limited to: Ceramide EOS, Ceramide NS, Ceramide NP, Ceramide EOH, Ceramide AS, Ceramide NH, Ceramide AP, Ceramide AH, Ceramide EOP, Ceramide NdS, Ceramide AdS, and/or Ceramide EOdS.

According to a further aspect of the invention, there is provided a composition comprising:
(a) A compound of formula (I), for example, urolithin A;
(b) Trehalose;
(c) Optionally an NAD precursor, for example, niacinamide; and
(d) A cosmetically-acceptable carrier.

According to a further aspect of the invention, there is provided a composition comprising:
(a) A compound of formula (I), for example, urolithin A;
(b) An NAD precursor, for example, niacinamide;
(c) Optionally trehalose; and
(d) A cosmetically-acceptable carrier.

In a further embodiment, there is provided a composition of the invention, for example, a cream or lotion, comprising about 0.8 to about 2.0% of urolithin A.

In a further embodiment, there is provided a composition of the invention, for example, a cream or lotion, comprising about 0.8 to about 1.5% of urolithin A.

In a further embodiment, there is provided a composition of the invention, for example, a cream or lotion, comprising about 0.8 to about 1.2% of urolithin A.

In a further embodiment, there is provided a composition of the invention, for example, a cream or lotion, comprising about 1% of urolithin A.

### Additional Components in Compositions of the Invention:

The compositions according to the invention may contain additional components beyond the urolithin, trehalose and/or NAD precursor. The additional components may be compounds that provide health benefits, for example selected from resveratrol, Co-enzyme Q₁₀, vitamin C, retinol, caffeine and amino peptides.

### Dosing

The effective amount of the composition to be taken will vary depending upon the manner of administration, the age, body weight, and general health of the subject. Factors such as the disease state, age, and weight of the subject may be important, and dosage regimens may be adjusted to provide the optimum response. For example, for a topical formulation, a dose of between 0.5 mg/cms to 10 mg/cm² administered in the morning and evening, for example, 2 mg/cm² administered in the morning and evening.

A representative topical composition is shown in Table 3:

TABLE 3
Representative formulation A:

| | | % |
|---|---|---|
| CAS | INCI Name (Mixed) | INCI |
| 7732-18-5 | AQUA (WATER) | 74.2 |
| 56-81-5 | GLYCERIN | 5.95 |
| 107-88-0 | BUTYLENE GLYCOL | 4.5 |
| 194043-92-0 | BUTYROSPERMUM PARKII (SHEA) BUTTER | 3.0 |
| 95912-86-0 | COCO-CAPRYLATE/CAPRATE | 2.6 |
| 31566-31-1 | GLYCERYL STEARATE | 2.15 |
| / | C15-19 ALKANE | 2.0 |
| / | Urolithin A | 1.0 |
| 6920-22-5 | 1,2-HEXANEDIOL | 1.0 |
| 1338-43-8 | SORBITAN OLEATE | 0.85 |
| 57-11-4 | STEARIC ACID | 0.8 |
| 9004-99-3 | PEG-100 STEARATE | 0.65 |
| 111286-86-3 | HYDROXYETHYL ACRYLATE/SODIUM | 0.32 |
| | ACRYLOYLDIMETHYL TAURATE | |
| | COPOLYMER | |
| 76050-42-5 | CARBOMER | 0.30 |
| 111-01-3 | SQUALANE | 0.22 |
| 1117-86-8 | CAPRYLYL GLYCOL | 0.2 |
| 1310-73-2 | SODIUM HYDROXIDE | 0.07 |
| 9005-67-8 | POLYSORBATE 60 | 0.06 |
| 70445-33-9 | ETHYLHEXYLGLYCERIN | 0.05 |
| 1406-66-2 | TOCOPHEROL | 0.045 |
| 71902-01-7 | SORBITAN ISOSTEARATE | 0.012 |
| 8001-21-6 | HELIANTHUS ANNUUS (SUNFLOWER) | 0.005 |
| | SEED OIL | |
| | | 100.0 |

### Treatments:

The composition of the invention can be taken as a single treatment or, more commonly, as a series of treatments. For a subject, a dose of the composition may, for example, be taken once, twice or three times per day, or one, two, three, four, five, six or seven times per week. It will also be appreciated that the effective dosage of the compound may increase or decrease over the course of a particular treatment.

### Medical and Non-Medical Treatments:

The compositions of the invention can be for use as a cosmetic or a medicament.

The term ‘about’ refers to a tolerance of ±20% of the relevant value, for example ±15% of the relevant value, such as ±10% of the relevant value or ±5% of the relevant value.

The phrase ‘extrinsic aging factors’ comprises factors relating to the environment such as sun exposure, smoking, hydration, diet, skin care products, and environmental pollution.

The phrase ‘intrinsic aging factors’ relates to the natural biological process of aging, including slower production of stem cells, estrogen loss, reduced collagen and elastin, and deteriorating facial tissues are all part of intrinsic aging. The natural biological processes of aging further include, decline in cellular function and metabolism, due to an impairment in metabolism or mitochondrial function.

The term ‘NAD’ refers to nicotinamide adenine dinucleotide.

The phrase ‘NAD precursor’ refers to any compounds, which are intermediates in the synthesis of NAD in vivo. Example of NAD precursors include: nicotinic acid (niacin), niacinamide (nicotinamide), nicotinamide riboside, tryptophan and nicotinamide mononucleotide.

The term ‘photoaging’ refers to the characteristic changes to skin induced by chronic ultra violet A and/or ultra violet B light exposure

The phrase ‘transepidermal water loss’ (TEWL) refers to the loss of water that passes from the inside of a body through the epidermal layer (skin) to the surrounding atmosphere via diffusion and evaporation processes.

A further advantage of the formulations and use of the invention is that compounds of formula (1), for example, urolithin A, are safe and non-irritant to the skin. Unlike some current skin treatment, for example, retinoids which have a number of side effects, for example, dryness and irritation, skin color changes, sensitivity to sunlight and redness, swelling, crusting, or blistering.

Further advantages of the formulations and use of the invention are that compounds of formula (1), for example, urolithin A, decrease skin redness, protect the skin from the sun, prevent photoaging, improve anti-wrinkle efficacy, improve skin hydoration, and/or improve collagen organization in the skin.

### Methods of Use

Certain compositions and methods described herein may have beneficial effects on skin. Certain compositions and methods described herein may treat and/or prevent skin disorders. Certain compositions described herein may be oral compositions to provide oral formulations for treating and/or preventing skin disorders. Certain compositions and methods described herein may improve and/or maintain an aesthetic appearance of skin.

Skin disorders that are treated include, but are not limited to, those caused by sun exposure, inflammation, and/or autoimmune diseases. Skin disorders that are treated may include erythematotelangiectatic rosacea, telangiectasias, papulopustular rosacea and/or phymatous rosacea.

i. Sun Exposure-Related Skin Disorders

Sun exposure-related skin disorders that are treated with the described compositions and methods include, but are not limited to, actinic keratoses, lentigines or age spots, seborrheic keratoses, sun burn, photosensitivity, moles, polymorphous light eruption, solar elastosis or wrinkles, skin cancer (such as melanoma, squamous cell carcinoma, basal cell carcinoma), and freckles.

ii. Inflammatory Skin Disorders

Inflammatory skin disorders that are treated with the described compositions and methods include, but are not limited to, psoriasis, contact dermatitis, atopic dermatitis, seborrheic dermatitis, asteatotic eczema, discoid eczema, hand eczema, gravitational/varicose eczema, eczematous drug eruptions, lichen simplex, acne, lichen planus, pityriasis lichenoides, keratosis lichenoides chronica, lichen nitidus, lichen striatus, mycosis fungoides, erythroderma, erythema multiforme, Stevens-Johnson Syndrome, vasculitis, and toxic epidermal necrolysis.

iii. Autoimmune Skin Disorders

Autoimmune skin disorders that are treated with the described compositions and methods include, but are not limited to, pyoderma gangrenosum, systemic lupus erythematosus, eosinophilic fasciitis, scleroderma, pemphigus vulgaris, bullous pemphigoid, alopecia areata, vitiligo, psoriasis, dermatomyositis, and dystrophic epidermolysis bullosa.

The present invention will be further understood by reference to the following non-limiting examples.

### EXAMPLES

The following Examples illustrate the invention.

Compounds

Urolithin A was prepared as follows:

Urolithin A (4) was prepared in two steps starting from 2-bromo-5-methoxybenzoic acid 1 and resorcinol 2. The pure compound was obtained as a pale yellow powder.

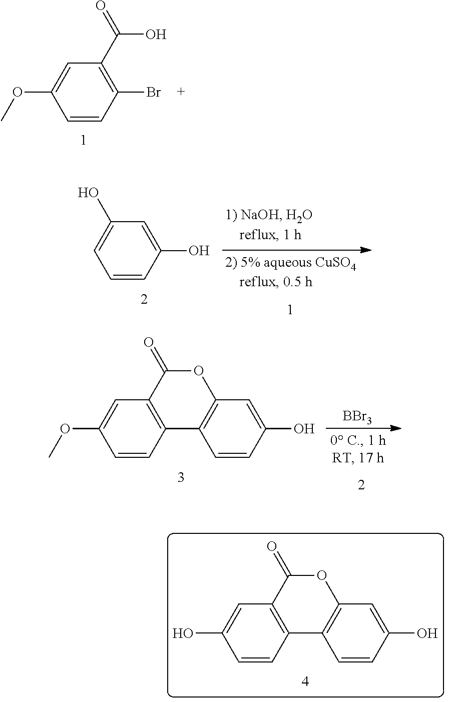

Step 1:

A mixture of 2-bromo-5-methoxybenzoic acid 1 (27.6 g; 119 mmol; 1.0 eq.), resorcinol 2 (26.3 g; 239 mmol; 2.0 eq.) and sodium hydroxide (10.5 g; 263 mmol; 2.2 eq.) in water (120 mL) was heated under reflux for 1 hour. A 5% aqueous solution of copper sulphate (3.88 g of CuSO₄·5H₂O in 50 mcarrierL water; 15.5 mmol; 0.1 eq.) was then added and the mixture was refluxed for additional 30 minutes. The mixture was allowed to cool to room temperature and the solid was filtered on a Buchner filter. The residue was washed with cold water to give a pale red solid which was triturated in hot MeOH. The suspension was left overnight at 4° C. The resultant precipitate was filtered and washed with cold MeOH to yield the title compound 3 as a pale brown solid.

Step 2:

To a suspension of 3 (10.0 g; 41 mmol; 1.0 eq.) in dry dichloromethane (100 mL) was added dropwise at 0° C. a 1 M solution of boron tribromide in dry dichloromethane (11.93 mL of pure BBr₃ in 110 mL of anhydrous dichloromethane; 124 mmol; 3.0 eq.). The mixture was left at 0° C. for 1 hour and was then allowed to warm up to room temperature. The solution was stirred at that temperature for 17 hours. Then ice was added thoroughly to the mixture. The yellow precipitate was filtered and washed with cold water to give a yellow solid which was heated to reflux in acetic acid for 3 hours. The hot solution was filtered quickly and the precipitate was washed with acetic acid, then with diethyl ether to yield the title compound 4 as a yellow solid. ¹H and ¹³C NMR were in accordance with the structure of 4.

### Example 1

PGE2 Release in Reconstituted Human Epidermis Under UV Stimulation

This study shows the impact of a moisturizing cream containing different concentration of Urolithin A (UA) on the secretion of the pro-inflammatory marker Prostaglandin E2 (PGE2) in the supernatant of reconstructed human epidermis.

Methods

Culture and Treatment

Methods

Ten-day-old reconstructed human epidermis (RHE) were placed in maintenance medium. The RHE were topically treated with or without moisturizing creams containing different concentration of Urolithin A active and incubated for 24 hours. The RHE were then rinsed in PBS and irradiated with UVB (+UVA)—850 mJ/cm² (+6 J/cm²) using a SOL500 Sun Simulator equipped with an H2 filter (Dr. Hönle, AG). After irradiation, the treatments were renewed and the RHE were incubated for 24 hours. In parallel, non-irradiated conditions were performed and kept in the dark during the irradiation time. All experimental conditions were performed in n=3. PGE2 released in the culture supernatants was measured using a specific ELISA kit according to the supplier's instructions (Enzo Life Sciences, ref. ADI-900-001). Statistical analysis performed by One-Way Anova.

| | | Low limit of | High limit of |
|---|---|---|---|
| ELISA Kit | Supplier | detection | detection |
| PGE₂ | Enzo Life Sciences, ref. | 39.1 pg/ml | 2 500 pg/ml |
| | ADI-900-001 | | |

Conditions

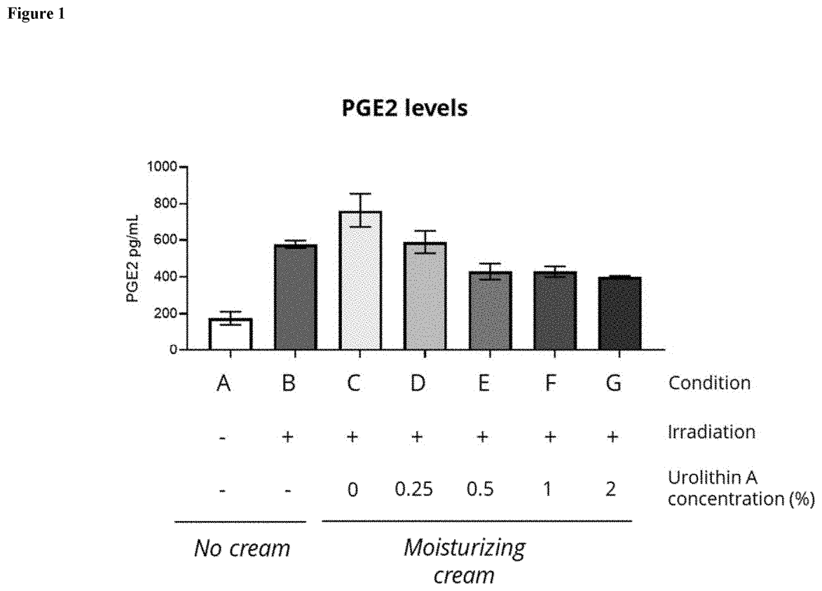

With reference to FIG. 1
A: non irradiated RHE without cream
B: irradiated RHE without cream
C: irradiated RHE with basic cream formulation (without Urolithin A)
D: irradiated RHE with cream containing Urolithin A at 0.25%
E: irradiated RHE with cream containing Urolithin A at 0.5%
F: irradiated RHE with cream containing Urolithin A at 0.1%
G: irradiated RHE with cream containing Urolithin A at 0.2%

Results

FIG. 1 summarizes PGE2 levels secreted by RHE, measured in the supernatant, i.e. the cell culture medium. Summary of statistics for all conditions is indicated below. Data indicate that:
a) UV irradiation significantly increases PGE2 levels
b) Interestingly the basic cream formulation (without urolithin A) induces significant increase in secreted PGE2
c) UA significantly reduces PGE2 secretion at concentrations of 0.5%, 1% and 2% compared to irradiated non-treated skin
d) UA significantly reduces PGE2 secretion at all concentrations tested, compared to irradiated skin treated with basic cream formulation without UA (0% UA).

| Comparison | Statistical method | Summary | p value |
|---|---|---|---|
| B vs. A | One-Way ANOVA | *** | 0.0007 |
| E vs. C | One-Way ANOVA | ** | 0.0036 |
| F vs. C | One-Way ANOVA | ** | 0.036 |
| G vs. C | One-Way ANOVA | **** | 0.0017 |
| E vs. B | Unpaired T-test | * | 0.0381 |
| F vs. B | Unpaired T-test | * | 0.0140 |
| G vs. B | Unpaired T-test | ** | 0.0010 |

Conclusions

Surprisingly, the application of a basic cream formulation has detrimental effects on skin protection against UV-induced inflammation. We observed an unexpected protective action of urolithin A on UV irradiated skin, blunting negative effects of basic cream, in addition to treating the effects of UV-irradiation.

### Example 2

Impact of Mitopure (Urolithin A) Containing Cream on Human Skin Aging after 8 Weeks of Application

Biological pathways and genes whose expression declines with age in human skin and that are upregulated following application of a cosmetic product containing Mitopure were identified.

Pathways (i.e. gene sets) and genes downregulated with skin aging have been extrapolated from a publicly available dataset in female twins in the age range of 39-85 years (Glass et al (2013) Genome Biology 3, 14: Article number: R752).

Pathways (i.e. gene sets) and genes upregulated with Mitopure have been identified from a clinical study in female subjects aged between 50 and 75 years. In this study, a cosmetic product with a 1% concentration of the active Mitopure or a placebo product were tested on skin aging for 8 weeks.

In both studies, skin biopsies have been collected and used to: (i) extract RNA, (ii) perform a high-throughput transcriptomics, i.e. gene expression analysis, (iii) perform a pathway enrichment analysis to identify gene set significantly modulated.

The gene set “collagen fibril organization” was found to be significantly downregulated in the skin aging study and significantly upregulated in the study following application of the Mitopure product.

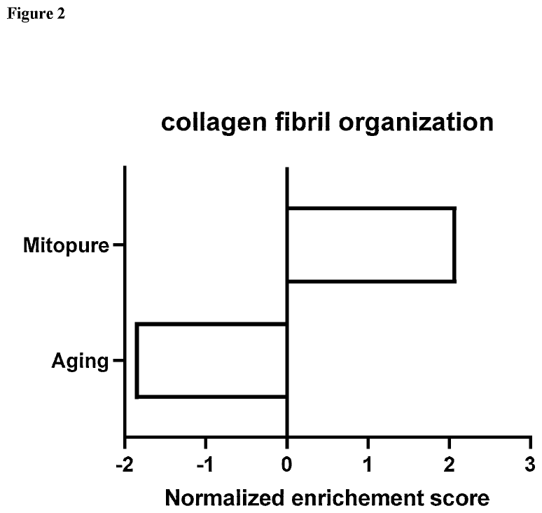

FIG. 2 represents the Normalized Enrichment Score (NES) for the “collagen fibril organization” gene set in both studies. NES indicates the degree of enrichment of a gene set. Negative values indicate that the pathway is downregulated; positive NES values indicate that the pathway is upregulated. NES of “collagen fibril organization” is negative in the skin study, indicating a decline of this gene set with skin aging. NES of “collagen fibril organization” is positive in the Mitopure study, indicating that application of Mitopure on human skin for 1 month at a concentration of 1% increases the same pathway that is reduced with skin aging.

The gene set “collagen fibril organization” is related to a biological pathway associated with skin firmness, strength and youthfulness. Genes belonging to this gene set “collagen fibril organization” are collagen genes encoding for collagen proteins. These genes include COL1A1, COL5A1, COL5A2, COL14A1, COL3A1.

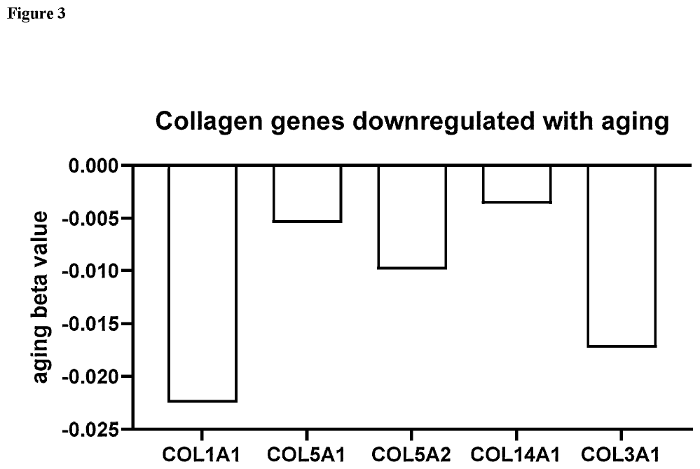

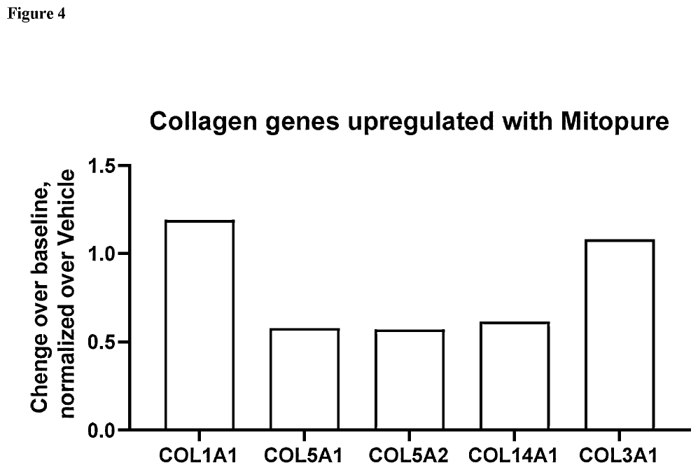

FIG. 3 shows that these genes are all downregulated in human skin with aging, as indicated by a negative aging beta score. Indeed, the aging beta score calculates how much gene expression changes with age in subjects of different ages, using a linear mixed model. Negative beta values indicate that these genes have reduced expression with aging in human skin.

The same collagen genes that are downregulated with skin aging are also all upregulated following Mitopure application, as shown in FIG. 4. The increased expression of these genes is calculated as change of expression comparing end of application to baseline, and values are normalized over the change in expression levels in the placebo control group.

Collagen genes encodes for collagen proteins that have a key role to support skin health with aging. Indeed, the dermis extracellular matrix consists mainly of collagen fibers (type I and type III), elastin and proteoglycans (Ross M H, Kaye G I and Pawlina W: Connective Tissue in Histology: A text and Atlas. Lippincott Williams & Wilkins; Pennsylvania, PA: 2003). The mechanical properties of the skin largely depend on the nature and organization of dermal collagen, that are responsible for the mechanical strength of the skin (Agache et al (1980) Archives of Dermatological Research volume 269, pages 221-232).

These results indicated that Mitopure improved expression of genes associated with collagen and assembly of collagen in human skin, that promote skin strength.

Methods

Clinical study, and sample processing and analysis was performed as described in Glass et al (2013) Genome Biology Article number: R75.

Gene set enrichment analysis (GSEA) was performed using the R package ClusterProfiler [Yu et al., 2012]. Briefly, in each comparison, genes were ranked by age beta score value, and the enrichment of a given gene set among up- or-down regulated genes was statistically tested. The minimum and maximum gene set sizes were set to 10 and 500, respectively. Resulting p-values were adjusted using the BH method. Terms with an adjusted p-value<0.05 were considered as statistically significant.

### Example 3

Impact of Urolithin a (Mitopure) on Human Primary Fibroblast and Keratinocyte Cells Results

In this experiment we compared the expression of genes associated with biological processes that are involved in skin homeostasis/health, in human primary keratinocytes treated with DMSO (control) of Urolithin A at either 2.5 uM or 10 uM.

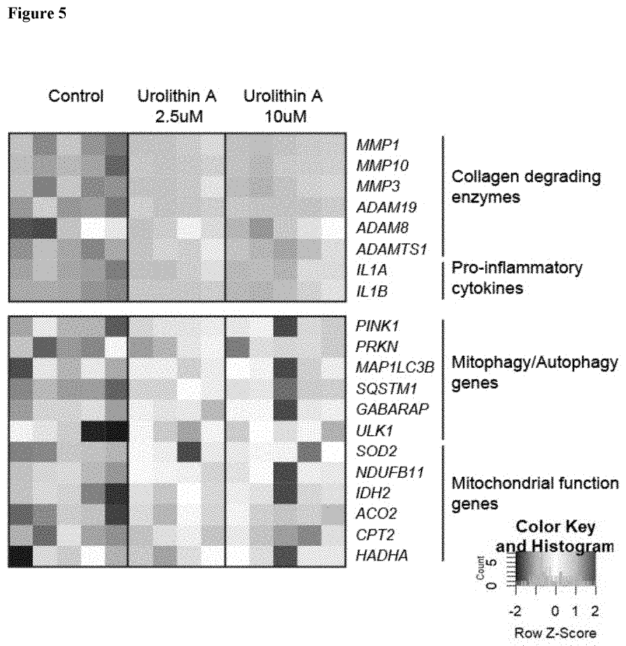

The heatmap in FIG. 5 indicates that UA supports the skin cells against detrimental effects of photoaging and environmental pollutants/toxins in human primary keratinocytes. In particular:
Urolithin A reduces expression of several genes encoding for enzymes involved in collagen degradation. These genes belong to category of Matrix Metalloproteinase (MMPs) and A disintegrin and metalloproteinases (ADAMs).
   MMPs are induced by UV skin irradiation and are causative of collagen degradation and skin photoaging (Quan et al (2013) J Invest Dermatol
   2013 May; 133(5): 1362-6). Indeed, it is assumed that around 80% of skin aging is caused by UV exposure, and most of the studies describing the alterations that affect ECM components refer to photoaging processes. In the case of collagens, these biochemical and molecular changes caused by photoaging are well defined, and one of their distinctive features is the reduction of the amount of fibrillar collagen mediated by different secreted proteases, such as MMPs (Rodríuquez et al (2017) Biochimica et Biophysica Acta—Molecular Cell Research 1864(11A), 2015-2025). MMP1 increase MMP-1 might be important in the skin-ageing effects of tobacco smoking (Lahmann et al (2001) Lancet 24; 357(9260), 935-6)
   ADAMs are upregulated in several skin disorders, in skin with impaired healing capacity, and after UV irradiation, i.e. photoaging (Kawaguchi & Hearing (2011) Enzyme Research Article 482498)
Urolithin A reduces expression of several genes encoding for proinflammatory cytokines, i.e. Interleukins (IL1A, IL1B). They are induced in skin exposed to UV and environmental pollutants (Kim & Park (2016) Molecular Mechanisms of the Aging Process and Rejuvenation. Editor Naofumi Shiomi. ISBN: 978-953-51-2568-6)
Urolithin A increases the expression of several genes encoding for proteins associated with improved autophagy/mitophagy and mitochondrial function. This is important as mitochondria are the primary organelle affected during chronological and UV-induced skin aging, the phenotypic manifestations of which are the direct consequence of mitochondrial dysfunction. (Sreedhar et al (2020) Cell Death & Disease 11, Article No: 444).

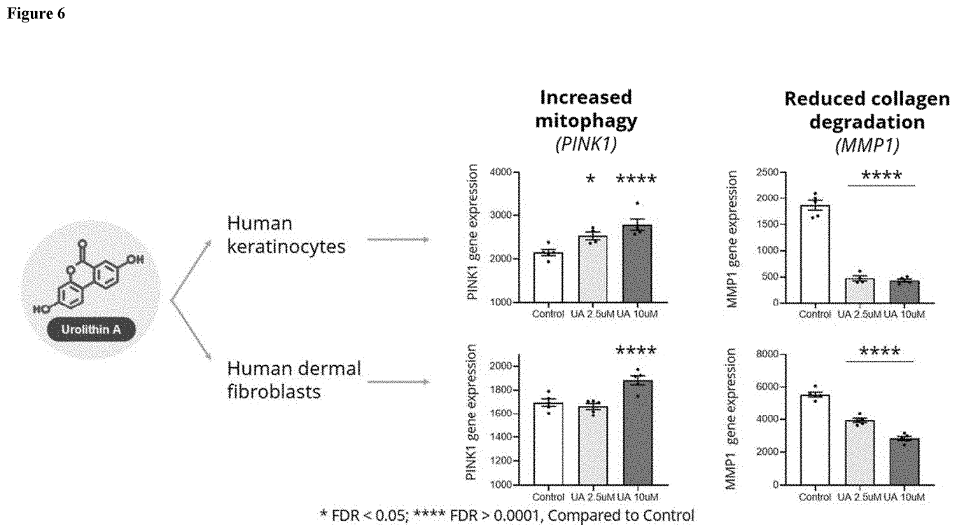

The FIG. 6 shows the expression levels of genes in both human primary keratinocytes and dermal fibroblasts treated with DMSO (control) of Urolithin A at either 2.5 uM or 10 uM. The data show that Urolithin A increased the mitophagy marker PINK1 in both cell types. And that Urolithin A decreases the matrix metalloproteinase MMP1, in both cell types, a marker that is increased and associated with skin chronological and photo aging.

Methods

Study was conducted on Normal Adult Human Dermal Fibroblasts (NHDF-Ad) from two pooled donors (Catalog #: CC-2511) and Normal Adult Human Epidermal Keratinocytes (NHEK-Ad) from two pooled donors (Catalog #: 00192627, Lonza). Cell batches are presented in the table below (Table 4).

The study was performed with five technical replicates for each experimental condition (FIG. 1).

TABLE 4
Skin cell donors' information

| Primary cells | Batch 1 | Batch 2 |
|---|---|---|
| NHDF-Ad | no 0000473428 | no 0000352805 |
| NHEK-Ad | no 0000520143 | no 0000280056 |

The primary skin cells were amplified in flask and seeded in 12-well cell culture plates, filled with 1 mL of culture medium and then incubated at 37° C. with 5% CO₂ and saturated hygrometry. The basic culture medium of human dermal fibroblasts was composed of FBM™ medium (Fibroblast Growth Basal Medium) supplemented with insulin 0.1%, hFGF-B 0.1%, GA-1000 0.1% and FBS 2%. The basic culture medium of human keratinocytes was composed of KBM™ Gold medium (Keratinocytes Basal Medium) supplemented with Hydrocortisone, Transferrin, Epinephrine, Gentamycin, Amphotericin B, BPE, hEGF and Insulin.

RNA Sequencing

Primary cells were treated at confluency (approximatively 250000 cells per well) with 0.1% DMSO (control), 2.5 μM and 10 μM of Urolithin A for 24 h, and harvested for RNA sequencing and expression profiling using the NGS platform. The significantly upregulated and downregulated mRNAs were analyzed with bioinformatic tools (FIG. 2).

RNA Extraction and Quality Control

RNA was extracted from the cell lysate using RNeasy Mini Kit (Qiagen) according to the manufacturer's protocol. Lysate buffer from the extraction kit was added to the cell pellet and sample stored at −80° C. until RNA extraction. Enzymatic DNase digestion was performed during extraction to remove DNA.

Upon extraction, RNA was quantified using Qubit™ RNA BR Assay Kit. RNA integrity was also evaluated with RNA ScreenTape and associated reagents by defining the RNA integrity number (RIN) and DV200 (percentage of fragments >200 nucleotides). RNA quantification results and RIN values were sent by email to the Sponsor, that validated the samples to use for library preparation.

Library Preparation and Sequencing

Libraries were prepared using Illumina Stranded mRNA Prep kit according to the manufacturer's protocol. Briefly, polyA RNA was captured and fragmented. cDNA was synthesized and index anchors were ligated. Libraries were then amplified and cleaned up. Prepared libraries were quantified using Qubit™ dsDNA BR Assay Kit and size was verified with Agilent High Sensitivity DNA Kit.

All libraries were normalized, pooled and denaturated. PhiX was included with the libraries as control for sequencing. The pool containing the libraries and PhiX were then loaded in the flow cell from the NextSeq 500/550 High Output Kit v2.5.

Data Processing and Analysis

Demultiplexing of raw sequencing data was performed with bcl2fastq software. Quality control of sequencing was performed with FastQC software. Reads were mapped to the Human genome (Hg38) and quality control metrics such as percent of uniquely mapped reads were produced. Exploratory data analysis was performed to verify the quality of the data. Data was normalized and differential gene expression analysis was performed to compare the biological groups of interest.

Data Acquisition, Processing and Analysis Software

The following systems were used for data electronic acquisition, processing and archiving.

| | | | Data |
|---|---|---|---|
| System | Used for | Version | format |
| Qubit 3.0 | RNA and library | Qubit 3.0 | Electronic |
| | quantification | | |
| TapeStation | RNA and library size | A.02.02(SR1) | Electronic |
| 2200 analyzer | evaluation | | |
| NextSeq 500 | Sequencing | NextSeq | Electronic |
| | | control | |
| | | software | |
| | | V2.2.0 | |
| Bcl2fastq | Demultiplexing | V2.20.0.422 | Electronic |
| FastQC | Control quality | 0.11.9 | Electronic |
| | sequencing data | | |
| ArrayStudio | Mapping of reads, | 11.6.05 | Electronic |
| | exploratory analysis, | | |
| | differential expression | | |
| | analysis | | |
| R | Exploratory graphical | 4.1.2 or above | Electronic |
| | analysis and differential | | |
| | expression analysis | | |

### Example 4

### Clinical Trial: Efficacy Study of a Novel Cosmetic Product to Reduce an UVB Induced Erythema

Objective

The aim of this exploratory study is to determine the erythema-reducing efficacy of a test product in two concentrations on a light sunburn induced by a sun simulator compared to an untreated control and a placebo product.

For this study female and male subjects with healthy skin on the back and an ITA°>28°.

A sunburn of 1.25 minimal erythemal dose (MED) was evoked on one set of test areas and 1.6 MED on the other set of test areas by irradiation with a sun simulator. On 3 test areas of each set products was applied 24 hours before irradiation, on 3 other test areas of each set products was applied immediately after irradiation. An irradiated but untreated test area served as a control. The effect of the test products was assessed by visual evaluation of erythema and instrumental color measurements by Chromameter before and after irradiation. The study was reviewed by an independent institutional review board (IRB) for ethical approval.

Efficacy Assessment

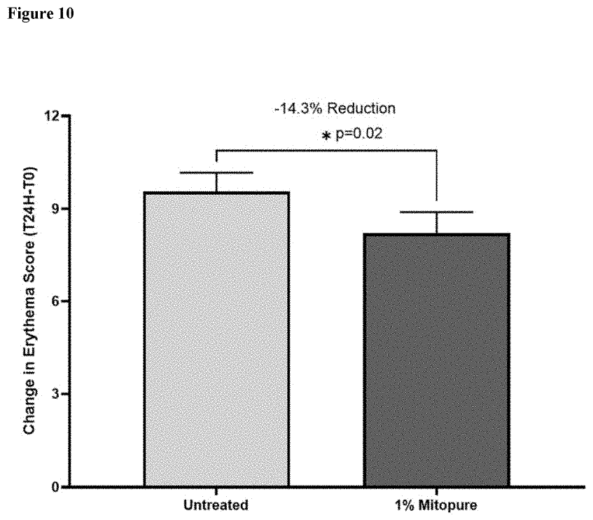

The following efficacy assessment(s) will be performed:
Erythema (a*) by Chromameter (See FIG. 10)
Visual erythema evaluation (trained grader)

Type of Product(s)

Face care product (two different concentrations of active (urolithin A) and placebo).

Study Design
Randomized
Double blind for Placebo and products
Placebo-controlled
Intra-individual comparison

Type of Statistics
Comparison to untreated

Assessment Times

Chromameter:
Baseline (before irradiation and application)
24 hours after irradiation

Visual Evaluation:
24 hours after irradiation

Test Materials

| Code | Product/Code/Sponsor/Concentration* |
|---|---|
| Irradiated with 1.25 | |
| MED | |
| A | Untreated area (Negative Control), irradiated with 1.25 MED |
| Pre-treated | |
| B | Product 1 (placebo or test product), pretreated and irradiated with |
| | 1.25 MED |
| C | Product 2 (placebo or test product), pretreated and irradiated with |
| | 1.25 MED |
| D | Product 3 (placebo or test product), pretreated and irradiated with |
| | 1.25 MED |
| Post-treated | |
| E | Product 1 (placebo or test product), irradiated with 1.25 MED and |
| | post-treated |
| F | Product 2 (placebo or test product), irradiated with 1.25 MED and |
| | post-treated |
| G | Product 3 (placebo or test product), irradiated with 1.25 MED and |
| | post-treated |
| Irradiated with 1.6 | |
| MED | |
| H | Untreated area (Negative Control), irradiated with 1.6 MED |
| Pre-treated | |
| I | Product 1 (placebo or test product), pretreated and irradiated with 1.6 |
| | MED |
| K | Product 2 (placebo or test product), pretreated and irradiated with 1.6 |
| | MED |
| L | Product 3 (placebo or test product), pretreated and irradiated with 1.6 |
| | MED |
| Post-treated | |
| M | Product 1 (placebo or test product), irradiated with 1.6MED and |
| | post-treated |
| N | Product 2 (placebo or test product), irradiated with 1.6 MED and |
| | post-treated |
| O | Product 3 (placebo or test product), irradiated with 1.6 MED and |
| | post-treated |

*Product 1, 2, 3 will be MITOPURE CREAM-18, MITOPURE CREAM-19 or MITOPURE CREAM-20. Creams will be formulated as set our in Representative formulation I above.

The test material(s) will be cosmetic products, as furnished by the Sponsor. Test materials will be used undiluted or diluted, as specified in the delivery form. The test materials will be stored at room temperature.

Test Area

Back of the subjects in the region between shoulder blade and hip. The area of the spine will be excluded.

Assignment of Test Areas

The application areas will be lined in four vertical rows (with twice 4 and 3 areas). Test products will be assigned to areas by cyclic permutation in each block of 3 test areas to minimize an anatomical selection bias The same assignment will be used for all four columns so that every product is on the same line on the back in all four columns. Pre-treated areas and post-treated areas are permuted in columns contralaterally on each side of the spine (area 1 to 4 and 11 to 14 or 5 to 7 and 8 to 10. The untreated area will be the upper or the lower area of the column with 4 areas (area 1 or 4 and area 11 or 14), but it will be centered in the middle of the two columns. Irradiation doses will be 1.25 on the right side (area 8 to 14) in subjects with even subject numbers and on the left side (area 1 to 7) in subjects with uneven numbers. Irradiation doses will be 1.6 on the other side.

Application Volume

75 μl

Application Mode

Test products will be applied to the assigned test areas on the erythemal spots using an epicutaneous patch test system (see ‘Patch Test System’).

Patch Test System

The following patch test system(s) will be used:
Occlusive patch test system: Finn Chambers®, large, backed on Scanpor®. In case of high fluidity of the test materials filter discs will be used.

Study Population

A minimum of 22 subjects will be recruited for this test from the general population and the neighboring communities of the Study Site's location so that about 20 subjects are expected to finish the study. Subjects who drop-out after randomization into the study are not replaced. All subjects will have a complete understanding of the test procedure.

All below mentioned inclusion, exclusion criteria and instructions for the subjects will be checked by a questionnaire before the start of the study and during the study. Conditions developing during the course of the study listed in the exclusion criteria and instructions as well as protocol deviations do not necessarily lead to the subject's exclusion. The investigator decides whether the subject is still eligible.

Inclusion Criteria
Written Informed Consent to participate in the study
Willingness to actively participate in the study and to come to the scheduled visits
Female and/or male
From 18 to 65 years of age
Uniform skin color and no erythema or dark pigmentation in the test area
ITA°>28 in the test area

Exclusion Criteria
Female subjects: Pregnancy or lactation
Drug addicts, alcoholics
AIDS, HIV-positive or infectious hepatitis
Conditions which exclude a participation or might influence the test reaction/evaluation
Participation or being in the waiting period after participation in cosmetic and/or pharmaceutical studies pertaining to the test area
One of the following illnesses with reduced physical capability/fitness: asthma (symptom-free allergic asthma is not an exclusion criterion), hypertension, cardiovascular diseases
Cancer not being diagnosed as cured and requiring chemotherapy, irradiation and/or hormonal treatment within the last 2 years
Insulin-dependent diabetes mellitus
Electronic implant (e.g., pace maker, insulin pump, hearing aid, and the like) that cannot be removed during irradiation
Documented allergies to cosmetic products and/or ingredients
Active skin disease at the test area
Irregularly tanned skin in the test area
Medical history of dysplastic nevi, melanoma or other skin carcinoma
Medical history of abnormal response to sunlight
Regular use of tanning beds (more than 10 times within the last 6 months)
Wounds, moles, tattoos, scars, irritated skin, excessive hair growth, etc. at the test area that could influence the investigation
Usage of medication with known photo-toxic and/or photo-sensitizing potential (e.g., some antibiotics, blood pressure regulating agents and antidepressants agents; Hypericum perforatum) within the last 14 days prior to the start of the study and/or throughout the entire course of the study
Any topical medication at the test area within the last 7 days prior to the start of the study and/or throughout the entire course of the study
Systemic therapy with immuno-suppressive drugs (e.g., corticosteroids) and/or antihistamines (e.g., antiallergics) within the last 7 days prior to the start of the study and/or throughout the entire course of the study
Systemic therapy with anti-phlogistic agents or analgetics (e.g., diclophenac), except for minor pain relief medicine like acetylsalicyiic acid or paracetamol within the last 3 days prior to the start of the study and/or throughout the entire course of the study

Instructions for Subjects
Instructions prior to the Start of the Study:
The subjects will be instructed not to . . .
   apply any leave on cosmetics (e.g., creams, lotions) to the test area within the last 3 days prior to the start of the study
   apply any detergents (e.g., soaps, shampoos, bath and shower products) to the test area in the morning prior to the start of the study
   smoke within the last 2 hours prior to the start of the study
   expose the test area to the excessive sunlight, UV-therapy and/or artificial tanning within the last 7 days prior to the start of the study
Instructions throughout the Course of the Study:
The subjects will be instructed not to . . .
   apply any leave on cosmetics (e.g., creams, lotions) to the test area throughout the entire course of the study
   apply any detergents (e.g., soaps, shampoos, bath and shower products) to the test area throughout the entire course of the study
   smoke within the last 2 hours prior to the instrumental measurements
   expose the test area to the excessive sunlight, UV-therapy and/or artificial tanning throughout the entire course of the study
   apply water to test areas (e.g., no baths, carefully showering, no visits to swimming

Test Procedure

Day 1:

The subjects will come to the Study Site. They will be informed about the study and give their written consent. The skin type of the subjects will be classified by use of colorimetric skin type classification.

Test areas will be outlined with a skin marker on the backs of the subjects. Thereafter, baseline measurements will be performed on all test areas with a Chromameter.

After that 6 spots for MED determination will be irradiated on the back with a diameter of 0.9 cm each. The UV-B dose will be increased from spot to spot by an increment of 25%.

Application will be performed on areas with pre-treatment (Codes B-D and 1-L).

Day 2:

Subjects will return to the study site 24±4 hours after irradiation. Reading of the minimal erythemal dose (MED) will be performed and the irradiation time for 1 MED will be determined. The correct dose for the irradiation of test areas will be calculated by multiplying the irradiation dose of 1 MED with the factor for irradiation (1.25 and 1.6).

Patches of the pretreated test areas (Code B-G) will be removed. Residues of the test products will be carefully removed with a dry soft paper tissue.

All test areas will be irradiated at least 1 hour after patch removal according to the factor for irradiation (1.25 or 1.6). After irradiation the test products will be applied to the irradiated spots which are to be treated post irradiation (Code E-G and M-O). An untreated irradiated control area will serve as negative control.

Day 3:

23±2 hours after irradiation subjects will return to the Study Site and patches will be removed. Residues of the test products will be carefully removed with a dry soft paper tissue. After a waiting period of at least 1 and not more than 2 hours the final visual assessments and Chromameter measurements will be performed.

Time deviations of ±10% are allowed for patch removal.

Test Schedule

A scheme of the test procedure is given as Appendix 1 to protocol.

Climatic Conditions

During the investigation(s) room temperatures without additional air conditioning will be used.

Instrumental Measurement(s)

The following instrumental measurement(s) will be performed:
CHROMAMETER CR 400 (Minolta, Device D-Langenhagen, Germany): Skin color can objectively be quantified by reflectance measurements with a tri-stimulus Chromameter. The measuring principle is based on the reflection of a Xenon flash that diffusely lightens the skin on an area of 8 mm in diameter. The vertically reflected light from the skin is then detected by high-sensitive silica photodiodes that provide a color analysis of the detected light. The Chromameter defines the measured color in the L*a*b* color coordinate system. The L*-value defines the brightness. The color is defined by the parameters a* (red-green axis; negative a*-value green, positive a*-value red) and b* (blue-yellow axis, negative b*-value blue, positive b*-value yellow). The a* value correlates well with visual assessments of skin redness (erythema), a* is a measure for erythema. An increase in the a*-value corresponds to an increase in the degree of skin redness.
   Parameter: a* Skin redness
   3 measurements per test area and assessment time
SOLAR SIMULATOR Multiport 300 W (SOLAR Light Co, Philadelphia, USA; equipped with appropriate filtration and lightguides): The system will be used with a UV-transparent flexible light fiber bundle to irradiate the test areas with the requested irradiation intensity on spots with a diameter of 1 cm. The irradiated spots may be irradiated at the same time. The spectrum is in accordance with the SPF test method of the IS024444.
   1 irradiation per test area

Visual Evaluation

At the above mentioned assessment times a trained grader will assess the skin erythema for each group of irradiation dose of the different test areas, according to the following scale:

Scoring Scale
−2=Marked increase in redness compared to negative control (untreated)
−1=Slight increase in redness compared to negative control (untreated)
0=No difference in redness compared to negative control (untreated) (no effect)
1=Slight decrease in redness compared to negative control (untreated)
2=Marked decrease in redness compared to negative control (untreated)
3=Complete suppression of redness

The untreated area will be set to 0.

Scores will be directly entered into a PC system with an appropriate computer program.

Expected Local Effect Due to Patch Test System

The use of tape to fix the chambers on the skin may lead to a tape reaction (erythema and pruritus). This is usually limited to areas of direct contact with the tape and fades usually within a few hours. Since this reaction is anticipated and considered a normal reaction after applying tape to the skin for a long period of time, tape reactions will not be documented.

In case of strong tape reactions that might have an influence on the scoring of the test areas, these reactions and the consequences upon the evaluation of the respective test areas will be documented.

Expected Local Effect Due to UV-Irradiation

The irradiation with UV-light of the skin may lead to sunburn (erythema). This is usually limited to areas of direct contact with the UV-light. This reaction is anticipated and considered a normal reaction after irradiation of a determined time. It will be documented in the study with the reading score. Only in case of unusual reactions, these reactions and the consequences upon the grades of the respective test areas will be documented.

Adverse Reactions

According to ICH GCP, adverse reactions (ARs) are defined as “all noxious and unintended responses to a medicinal product related to any dose”. For clinical studies with cosmetics, consumer products or medical devices the following interpretation is adopted: The phrase “responses to a medicinal product” means that a causal relationship between the test material and an adverse event (AE) is at least a reasonable possibility, i.e. the relationship cannot be ruled out. Therefore, an AE which the physician classifies as having a causal relationship to the test material of at least ‘possible’ (i.e. possible, probable) is defined as an AR.

All adverse reactions (excluding those parameters being scored as part of the protocol) will be documented in the study records. The obligation to document ARs starts with enrolment of the subject in a study until 5 days following last administration of test product (if reported by the subjects).

In case of ARs it is the responsibility of the investigator to inform the Sponsor within 3 working days the latest. ARs will also be reported in the final report.

Analysis of Data

For this study the following analysis will be defined:

Statistical Data Analysis

A significance level of 0.05 (alpha) will be chosen for statistical analysis. Due to the explorative character of this study, no adjustments for multiplicity will be performed.

Instrumental Measurements:

Pairwise comparison of treatments will be done by paired t-Test on differences to baseline
within each of the four groups: (A,B,C,D); (A,E,F,G); (H,I,K,L) and (FI,M,N,0)
between pre/post within erythemal dose: (B,E); (C,F); (D,G); (1,M); (K,N); (L,0)
between erythemal dose: (A,H); (B,1); (C,K); (D,L); (E,M); (F,N); (G,0).

Visual Evaluation:

Pairwise comparison of treatments will be done by Wilcoxon signed rank test on raw data
within each of the four groups: (A,B,C,D); (A,E,F,G); (H,I,K,L) and (H,M,N,0)
between pre/post within erythemal dose: (B,E); (C,F); (D,G); (1,M); (K,N); (1,0)
between erythemal dose: (A,H); (B,1); (C,K); (D,L); (E,M); (F,N); (G,0).

The computation of the statistical data will be carried out with a commercially available statistics software (SAS for Windows).

### APPENDIX 1 TO PROTOCOL

| | Day 1 | Day 2 | Day 3 |
|---|---|---|---|
| Informed Consent | X | | |
| In-/Exclusion Criteria | X | | |
| Visual Evaluation (Erythema) | | | X |
| Instrumental Measurements | X | | X |
| (Chromameter) | | | |
| Irradiation for MED | X | | |
| determination | | | |
| Evaluation of MED | | X | |
| Irradiation with 1.25 and 1.6 | | X | |
| MED | | | |
| Application of Test Product(s) | X (areas | X (test areas | |
| | with pre- | with post- | |
| | Treatment) | treatment | |
| Objective Visual Evaluation | | | X |
| (by a trained gender) | | | |

Results

As expected, all erythema (a*)-values measured 24 hours after irradiation were higher than at baseline for all treatment codes, thus indicating the development of a sunburn on all the tested areas. From this study, the Chromameter measurements showed significantly less skin redness for skin creams containing 1% Urolithin A, at the 1.6 MED UVB dose in comparison to the respective untreated area (p=0.02; FIG. 10). The skin redness/erythema was reduced by approximately 14.3%. This is in line with the observed reduction on pro-inflammatory mediators (PGE2 levels) observed in FIG. 1 on reconstituted human epidermis following UVB exposure. Skin creams containing doses of Urolithin A at 0.5% or placebo did not show any significant effect on the skin redness/erythema in this clinical study

### Example 5 (Aging Study 1)

Clinical Trial: Evaluation of the Anti-Aging Efficacy of a Novel Cosmetic Product Containing Urolithin a Only

1.1 Objective

The aim of this proof of concept study is to investigate the effect of cosmetic products with two different concentrations of the active on skin aging and in acting on the mitochondrial effect in comparison to a placebo product.

For this study female subjects aged between 50 and 75 years with visible wrinkles in the face will be enrolled.

To analyze mitochondrial effects biopsies will be taken from a subpanel on volar forearms prior and after 8 weeks of product application and will be analyzed for biomarkers by 3rd parties (See FIGS. 2 and 4).

The effect of the test product on the skin barrier function will be assessed by transepidermal waterless (TEWL) at the beginning as well as after 2 weeks and after 8 weeks of product application. Furthermore, effects on the facial skin on skin hydration (by Corneometer), skin elasticity and firmness (by Cutometer) and skin color (by Spectrophotometer) will be investigated. Additionally, skin layer thickness and undulation index of the dermal epidermal junction (DEJ) will be measured by line-field confocal optical coherence tomography (LC-OCT). Images for wrinkle analysis and for documentation purpose will be taken with Colorface. A questionnaire regarding product traits will be filled in after 2 weeks of product application and at the end of the study.

Main Endpoints:
Change from baseline in skin mitochondrial health (assessed on skin biopsies) after 8 weeks
Change from baseline in skin barrier function (assessed on transepidermal waterless) after 8 weeks

Additional Outcomes:
Change from baseline in skin barrier function (assessed on transepidermal waterless) after 2 weeks
Change from baseline in skin hydration (assessed on skin capacitance) after 2 and 8 weeks
Change from baseline in pigmentation (assessed on skin color) after 2 and 8 weeks
Change from baseline in skin firmness after 2 and 8 weeks
Change from baseline in skin elasticity after 2 and 8 weeks
Change from baseline in skin layer thickness and undulation index of DEJ (assessed on LC OCT Image analysis) after 8 weeks for both groups or after 2 and 8 weeks for only one group*
Change from baseline for wrinkles and fine lines (assessed on Colorface Image analysis) after 2 and 8 weeks
Subjective Evaluation of product traits assessed via questionnaire after 2 and 8 weeks
upon decision of the sponsor, depending on the results of the other instrumental measurements

1.2 Efficacy Assessment

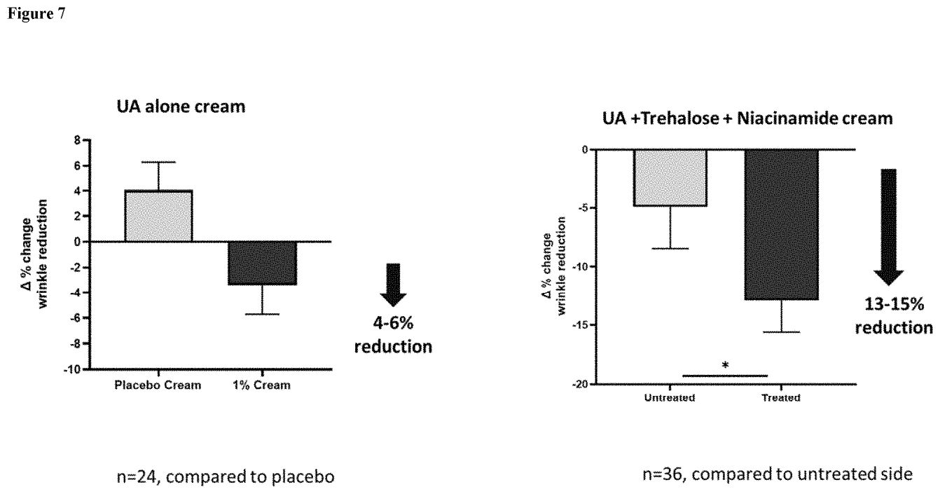

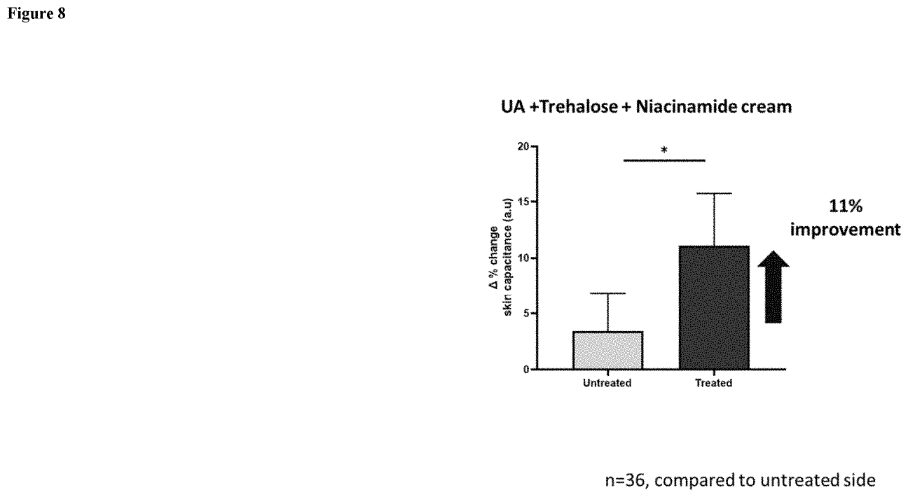

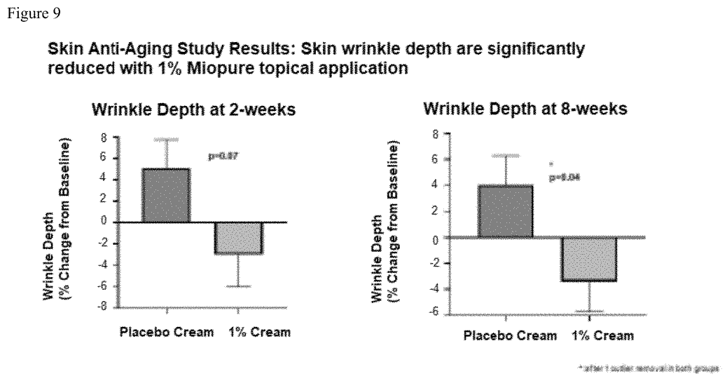

The following efficacy assessment(s) will be performed:
Biomarker analysis of 3 mm punch-biopsies on 30 subjects (15 subjects per group) by Sponsor/3^{rd} party
Transepidermal Water Loss (TEWL) by Tewameter [g/(m2h)j
Skin Hydration—Skin Capacitance by Corneometer [a.u.j] (see FIG. 8)
Skin Color by Spectrophotometer
Skin Firmness—R0 (Uf) by Cutometer [mm]
Skin Elasticity—R7 (Ur/Uf) by Cutometer
Image analysis by line-field confocal optical coherence tomography (LC-OCT) by Damae**:
(a) Skin layer thickness (epidermis and stratum corneum)
(b) Undulation index of the dermal epidermal junction (DEJ)
   Image analysis of Colorface Images by Newtone
(a) analysis of wrinkles and fine lines (see FIG. 7)
(b) creation of average face
   Questionnaire of product traits
      upon decision of the sponsor, depending on the results of the other instrumental measurements analysis will be done on either one group at three timepoints, or on two groups at two timepoints

1.3 Tolerability Assessment
Not applicable

1.4 Type of Product(s)

Face care product (two different concentrations of active (urolithin A) and placebo)

1.5 Study Outline
Exploratory
Randomized
Double blind
Intra-Individual Comparison (before and after treatment)
Placebo controlled

1.6 Type of Statistics
Comparison between treatments
Comparison between assessment times

1.7 Assessment Times
Baseline (Day 1)
Day 15 (After 2 weeks of application)
Day 57 (After 8 weeks of application)

2 Test Materials

| Study Group | Code | Product/Code/Sponsor/Concentration |
|---|---|---|
| Group 1 | A | MITOPURE CREAM-18 or MITOPURE |
| | | CREAM-20 |
| | B | MITOPURE CREAM-18 or MITOPURE |
| | | CREAM-20 |
| Group 2 | C | MITOPURE CREAM-19 or MITOPURE |
| | | CREAM-20 |
| | D | MITOPURE CREAM-19 or MITOPURE |
| | | CREAM-20 |

Mitopure = Urolithin A
Mitopure cream 18 = 1% Mitopure
Mitopure cream 19 = 0.5% Mitopure
Mitopure cream 20 = Placebo

The test material(s) will be cosmetic products, Test materials will be used undiluted or diluted, as specified in the delivery form. The test materials will be stored at room temperature.

2.2 Test Area

Face and volar forearms (for biopsies)(See FIG. 23)

2.3 Assignment of Test Areas

Area 1 is located on the right side of the face, Area 2 on the left side. Area 1 and 2 will be used for instrumental measurements. For biopsy subpanel area 3 is located on the right forearm, area 4 on the left forearm

Test products will be randomly and balanced assigned to the right and left side of the face (and to right and left volar forearm for biopsy subpanel). For biopsy subpanel treatment codes allocation (right and left) will be the same for face and volar forearms.

Each group of 24 subjects will receive one of the test product and the placebo.

15 subjects of each group will be chosen randomly to be included into the biopsy subpanel.

2.4 Application Volume

Approximately 2 mg/cm2

Face: Amount needed for a half face (including eye area) corresponds to approximately a hazelnut size amount of test product.

Volar forearm: Amount needed for each volar forearm corresponds to approximately a pea size amount of test product.

2.5 Application Mode and Frequency

If the test product(s) are used by the subjects themselves appropriate instructions will be given to the subjects orally and if deemed necessary in writing.

The correct amount of the product to be applied will be demonstrated by a technician during the first application on Day 1. The test material(s) will then be applied twice daily in the morning and evening for 8 weeks by the subjects at home, according application training.

The subjects will be instructed to use the test product every day in the morning and evening. On Day 15, subjects will be instructed not to apply the test products in the morning before visiting the study center, the morning application will take place at the site after all assessments.

2.6 Accountability and Destruction

The responsibility for the test product(s) accountability at the study site rests with proderm. Records of the test product's receipt and disposition of unused product(s) or alternative return to the sponsor will be maintained, proderm ensures that the test product(s) will be used as directed by this protocol. Test material remaining at the conclusion of the study will be destroyed at least 6 weeks after issuance of the final report unless requested otherwise.

2.7 Duration of Treatment

8 weeks per subject

2.8 Post-Treatment Product

A post-treatment product (sunscreen for sensitive skin, SPF 50+) will be used to the subpanel with biopsies after the end of study conduct to be used when exposing the biopsy wounds to sunlight.

3 Study Population

3.1 Subject Numbers

A minimum of 48 subjects (24 subjects for each group) will be recruited for this test from the general population and neighboring communities of the Study Site's location so that about 40 subjects (20 per group) are expected to finish in the study. Subjects who drop-out after randomization into the study will not be replaced. All subjects will have a complete understanding of the test procedure.

3.2 Selection of Subjects

All below mentioned inclusion, exclusion criteria and instructions for the subjects will be checked by a questionnaire before the start of the study. Conditions developing during the course of the study listed in the exclusion criteria and instructions as well as protocol deviations do not necessarily lead to the subject's exclusion. The investigator decides whether the subject is still eligible.

The subjects will be instructed to inform the study site in case of medical problems or changes in therapies.

Inclusion Criteria
Written Informed Consent to participate in the study
Willingness to actively participate in the study and to come to the scheduled visits
Female
From 50 to 75 years of age
Visible wrinkle in the face (grade 3 to 6 according to proderm scale) see Appendix 2
Healthy skin in the test areas
Vaccination of tetanus within the last 10 years (for biopsy sub group)

Exclusion Criteria
Female subjects: Pregnancy or lactation
Drug addicts, alcoholics
AIDS, HIV-positive or infectious hepatitis
Conditions which exclude a participation or might influence the test reaction/evaluation
Participation or being in the waiting period after participation in cosmetic and/or pharmaceutical studies pertaining to the test area
Cancer not being diagnosed as cured and requiring chemotherapy, irradiation and/or hormonal treatment within the last 2 years
Diabetes mellitus (type 1 and 2)
One of the following illnesses with reduced physical capability/fitness: asthma (symptom-free allergic asthma is not an exclusion criterion), hypertension, cardiovascular diseases
Documented allergies to cosmetic products and/or ingredients, skin care and/or skin cleansing products
Intolerability against adhesive dressing (e.g., acrylate)
Active skin disease at the test area
Regular use of tanning beds
Wounds, moles, tattoos, scars, irritated skin, excessive hair growth, etc. at the test area that could influence the investigation
Any topical medication at the test area within the last 3 days prior to the start of the study
Medical treatment for wrinkle reduction (e.g., peeling with vitamin A or fruit acids) on the face within the last 2 weeks prior to the start of the study
Systemic therapy with immuno-suppressive drugs (e.g., corticosteroids) and/or antihistamines (e.g., antiallergics) within the last 7 days prior to the start of the study
Systemic therapy with anti-phlogistic agents or analgetics (e.g., diclophenac), except for minor pain relief medicine like paracetamol within the last 3 days prior to the start of the study
Therapy with antibiotics within the last 2 weeks prior to the start of the study
Regular medication with anti-coagulating drugs like Aspirin®, Macumar®, etc. (e.g., for thrombosis prophylaxis) within up to 15 days prior to the taking of the biopsies
Past cosmetic surgery procedure in the test area (e.g., laser, facelift)
History of complications at wound healing (e.g., keloids, hypertrophic scars or contracture scar)

Instructions Throughout the Course of the Study:

The subjects will be instructed not to . . .
smoke within the last 2 hours prior to the instrumental measurements
drink any caffeinated beverages (e.g., coffee, cola, black tea) within the last 2 hours prior to the instrumental measurements
bring the test area in contact with water (e.g., no bathing and swimming, only careful showering excluding the test area) within the last 2 hours prior to the instrumental measurements
apply any products with the same indication as the test product to the test area throughout the entire course of the study
apply any leave on cosmetics (e.g., creams, lotions) to the test area throughout the entire course of the study
apply any decorative cosmetics (e.g., mascara, eye shadow, make-up, powder and other cover products) to the test area on the scheduled visits
remove the hairs (e.g., shaving, epilation, waxing) in the test area throughout the entire course of the study
expose the test area to the excessive sunlight, UV-therapy and/or artificial tanning throughout the entire course of the study
apply water to test areas after sampling of biopsies until healing of the wounds (e.g., no baths, carefully showering, no visits to swimming pools, no sauna)

The subjects will be instructed to . . .
use the provided sun cream on the wounds for the following 8 weeks after healing of the wounds, when exposing the test area to artificial or natural UV-light
wear a wound dressing on the biopsy wounds for about five days after biopsy sampling
wear a long-sleeved shirt or a hat when exposing the test areas to intense sunlight throughout the entire course of the study
perform the last application of the test product(s) on the previous evening prior to the scheduled visits
apply the test product(s) to the test areas with the following frequency (according to proderm's instructions): 2× daily throughout the entire course of the study
continue to use the normally used detergents (e.g., soaps, shampoos, bath and shower products) in the test area, but not to change the brand or use new products throughout the entire course of the study
continue to use the normally used decorative cosmetics (e.g., mascara, eye shadow, make-up, powder and other cover products) in the test area, but not to change the brand or use new products throughout the entire course of the study

5 Test Procedure

5.1 Description of Test Procedure

Screening:

The subjects will come to the Study Site. They will be informed about the study in both oral and written form and give their written consent. The suitability of each subject will be evaluated according to the inclusion/exclusion criteria. Prior/concomitant diagnoses and prior/concomitant therapies relevant for the study will be recorded. Subjects fulfilling the inclusion/exclusion criteria will be enrolled in the study.

Day 1: The subjects will come to the Study Site, after acclimatization period images will be taken and instrumental measurements (baseline) on the test areas will be performed. For the biopsy subpanel biopsies will be taken on both volar forearms.

The test product(s) will be issued to the subjects with instructions to use them on the assigned test areas and a diary to document the product application will be handed out. The assignment of test product(s) will be done according to a randomization scheme provided by proderm. The first product application will be performed under the guidance of a technician. One further application will be performed by the subjects at home.

Day 2 to 14: Subjects will use the test product(s) as described above (see ‘Application Mode’) and will document the product application in the diary. The last product application will be performed in the evening before the next visit at the Study Site.

For biopsy subpanel: In case the biopsy wounds will not heal until Day 8 they will be instructed to inform the study site.

Day 15: Subjects will return to the Study Site. After acclimatization period images and instrumental measurements on the test areas will be repeated. The diary will be checked by a technician, a questionnaire regarding product traits will be filled in by the subjects and the biopsy wounds will be followed up if applicable. Afterwards product application will be performed under the guidance of a technician. One further application will be performed by the subjects at home.

Day 16 to 56: Subjects will use the test product(s) as described above (see ‘Application Mode’) and will document the product application in the diary. The last product application will be performed in the evening before the next visit at the Study Site

Day 57: Subjects will return to the Study Site. After acclimatization period images and instrumental measurements on the test areas will be repeated and the subjects will fill in a questionnaire concerning test material traits of the test product. The remaining test materials and the diary will be returned. For the biopsy subpanel biopsies will be taken again on both volar forearms

Follow up visit for biopsy subpanel (7 of ±2 day after last biopsy sampling) The subjects will return to the Study Site for follow-up of the wounds. A sunscreen will be provided to the subjects with instruction to use the sunscreen on the test areas for the following 8 weeks

A deviation of ±2 days will be accepted, since no substantial influence on the outcome of the study is expected.

5.2 Climatic Conditions

The instrumental measurement(s) will take place in an air-conditioned room at a temperature of 22+2° C. and at 50±7.5% relative humidity. Before measurements the subjects will stay in the climatized room for at least 30 minutes.

5.3 Test Schedule

A scheme of the test procedure is given as Appendix 1 to protocol.

5.4 Instrumental Measurement(s)

The following instrumental measurement(s) will be performed:

TEWAMETER® TM 300 (Courage & Khazaka, Cologne, Germany): Transepidermal water loss (TEWL) is a non-invasive method to measure the barrier function of the skin and is regarded as a sensitive parameter to quantify skin barrier damage. Briefly, the insensible water evaporation from the skin is measured by placing cylindric open chamber with two hygrosensors at defined distance to the skin. The probe is held in place for each measurement for 30 seconds. This is to assure that a stable value has been established. The first part of the measurements belongs to this equilibration phase. The values of the last 10 seconds (=10 values) are averaged as the actual measurement value.

−1 measurement per test area and assessment time

SPECTROPHOTOMETER (CM 700d, Minolta, Langenhagen, Germany): The skin reactions are objectively quantified using reflection measurement with a tri-stimulus spectrophotometer. The spectrophotometer is capable of reflectance color measurements in CIE L*a*b* units (CIE 1976). The measuring principle is based on the reflection of a Xenon flash that lightens the object diffusely and uniformly.

The CIE L*a*b* color space is device independent. The L* value defines the brightness. The color is defined by the parameters a* (red-green axis; negative a* value: green; positive a* value: red) and b* (blue-yellow axis, negative b* value: blue; positive b* value: yellow).

−3 repetitive measurements per test area

CUTOMETER MPA 580 (Courage & Khazaka, Cologne, Germany). The measuring principle is based on suction of the skin into an aperture of 2 mm in diameter. In the measuring head, a defined vacuum of 300 mbar is induced. The measurement head is placed on the skin with a defined pressure and the skin is sucked into the opening of the measuring head for 5 seconds with a subsequent measuring period of another 5 seconds after release. Using an optical measuring system, it is detected contactless how far the skin is sucked into the measuring head; this value and the skin's ability to return to its original state gives a measure for the elasticity of the skin.

Parameters:

R0 Total elasticity (=Uf=first max. amplitude, highest point of the first curve,)

R7 Quotient of elastic relaxation to total elasticity (=Ur/Uf=portion of the elasticity compared to the complete curve, the closer the value is to 1 (100%) the more elastic the curve

−1 measurement per test area and assessment time

CORNEOMETER CM 825 (Courage & Khazaka, Cologne, Germany): The measurement of stratum corneum hydration will be performed by the electrical capacitance method with the corneometer. The measuring principle is based on changes in the capacitance of the measuring head, functioning as a capacitor. Between the conductors consisting of gold, an electrical field is built. By these means, the dielectricity of the upper skin layer is measured. Because the dielectricity varies as a function of the skin's water content, the stratum corneum hydration can be measured.

−5 measurements per test area and assessment time

LC-OCT 800 3D (DAMAE Medical, Paris, France): Line-Field Confocal Optical Coherence Tomography is a technique that combines the principles of time-domain optical coherence tomography and confocal microscopy. With this technique skin can be imaged in vivo in its native state without further preparation. This method enables an in vivo mapping of the skin through measuring the echo-time delay and amplitude of light backscattered from cutaneous microstructures through low-coherence interferometry associated with confocal spatial filtering. The different layers of the epidermis, the dermis and the dermal-epidermal junction can be clearly imaged using a live vertical, live horizontal and 3D mode. For the 3D mode a 3D stack of size of 1.2 mm×0.5 mm×0.5 mm is generated with a resolution of 1.3 pm×1.3 pm×1.1 pm.
Within each test area, two 3D acquisitions will be assessed.

Parameter:
Skin layer thickness (epidermis and stratum corneum)
Undulation index of the dermal epidermal junction

COLORFACE® PHOTOBOX (Newtone Technologies, Lyon, France): Colorface® is used for standardized, computer-controlled facial photography. It is equipped with a 24 Mpixel Nikon camera and a various range of LED-flashes at fixed positions in the booth. Illumination of the face is done with
[symbol] standard flashes
[symbol] parallel-polarized light
[symbol] cross-polarized light
□ UV flashes
   to obtain fluorescent images. The acquisition software for Colorface is used as image database, but also defines the settings for the respective study. All settings are fixed so that unwanted changes of image capture parameters can be avoided. The instrument enables facial photography of high quality and same illumination at different assessment times. Images of the following position of the face will be taken:
[symbol] front
[symbol] right
[symbol] left
   1 single image per setting, face position and assessment time
   Data will be stored in the Newtone Imaging access cloud
   Images will be provided to the sponsor in an anonymized way (e.g., black bar covering eyes) if possible.

5.5 Investigational Method(s)

The following investigational method(s) will be performed:

BIOPSY EXCISION: Rooms used for taking of biopsies will be disinfected. The subject is asked to lie on a bench in an appropriate position to ensure the biopsy area is accessible. A Medical Specialist cleanses the area of skin where the biopsy will be performed, with the disinfectant spray (e.g., Octenisept®). A local anesthetic (e.g., Skandicain® 1%) will be administered to the site subcutaneously using a sterile syringe, fitted with a suitable sterile needle, to the biopsy area prior to excision of the biopsy. Punch biopsies (3 mm) will be sampled by a physician using a punch biopsy instrument. The biopsy will contain the epidermis and the dermis. Each biopsy sample will be appropriately labelled (e.g., study number, subject's number).

With this procedure only small wounds are induced. With this procedure only small wounds are induced. Following the biopsy excision, the wound will be dosed closed with SteriStrips®, or if necessary, be sutured with stitches and covered with a protective dressing. The subjects will be supplied with waterproof dressings for covering the wounds after skin biopsies were taken. The wounds have to be kept dry until the wounds are healed. Instructions on changing the dressing and spare dressings are provided to the subjects.

A hyperpigmentation after healing of this lesion is likely. The patients will have knowledge about that and further possible side effects. In order to reduce the risk of hyperpigmentation the patients will be instructed to avoid sunlight at the test areas after conduct of the study and/or to use a sun protection cream.

Number of Biopsies
Day 1 and Day 57: 1 biopsy per test area and assessment time (4 biopsies in total per subject)

Handling of Biopsies
After biopsy sampling the biopsy will be cut into two parts and the parts will be stored separately in a sterile Eppendorf tube. One part is collected in the tubes without buffer; the second part is stored in tubes in 1 ml of RNA-later (RNAlater™ Solution AM7024, Thermo Fisher Scientific) buffer, to prevent RNA degradation.
Samples are snap frozen on liquid nitrogen until further processing, stored on dry ice until the end of the study day and at −80° C. until shipment.
To minimize sample cross-contamination, a fresh pair of gloves will be worn by the person preparing the samples

Storage and Shipment of Biopsies

The biopsy material will be stored at −80° C. The shipment of samples will be performed on dry ice with an appropriate courier service (World Courier) after the end of the study (on the next working day, if not requested otherwise)

Analysis of Biopsies (Further Step without Subject Contact)

Analysis of biopsies will be performed by the Sponsor and selected 3rd parties.

The following evaluations will be performed:
Transcriptomics of skin biopsies
Analysis of protein biomarkers of cellular health

Gene set enrichment analysis (GSEA) will be employed to look for overrepresentation of known mitochondrial pathways and gene functional categories among the regulated genes expressed in human skin. Genes will be ranked by fold change (high to low) and filtered (unadjusted P value of 0.1 before using them) in the GSEA analysis to reduce the risk of false positives. Gene sets defined by the Gene Ontology (GO) Consortium (http://www.geneontology.org/) will be analyzed.

5.6 Image Analysis

Analysis will be done by an external partner and a separate report will be prepared by the external partner (Newtone).

The Colorface images of test areas will be evaluated with regards to the following:

Crow's Feet Analysis:
Parameter: Visible length, Visible surface, Visible depth and Visible volume
Illumination: STD60
View: profile: right and left
Area: Crow's Feet

Average 2D Face:

One average face per group

1 crow's feet application on each hemi-face (of the best case per group chosen by the sponsor, 1 view, face or profile to D1, D15 and D57

5.7 Questionnaire for Product Acceptance by Subjects

Product traits will be assessed by the subjects and will consist of closed questions with predefined identical options to tick.
−2=Fully disagree
−1=Rather disagree
1=Rather agree
2=Fully agree

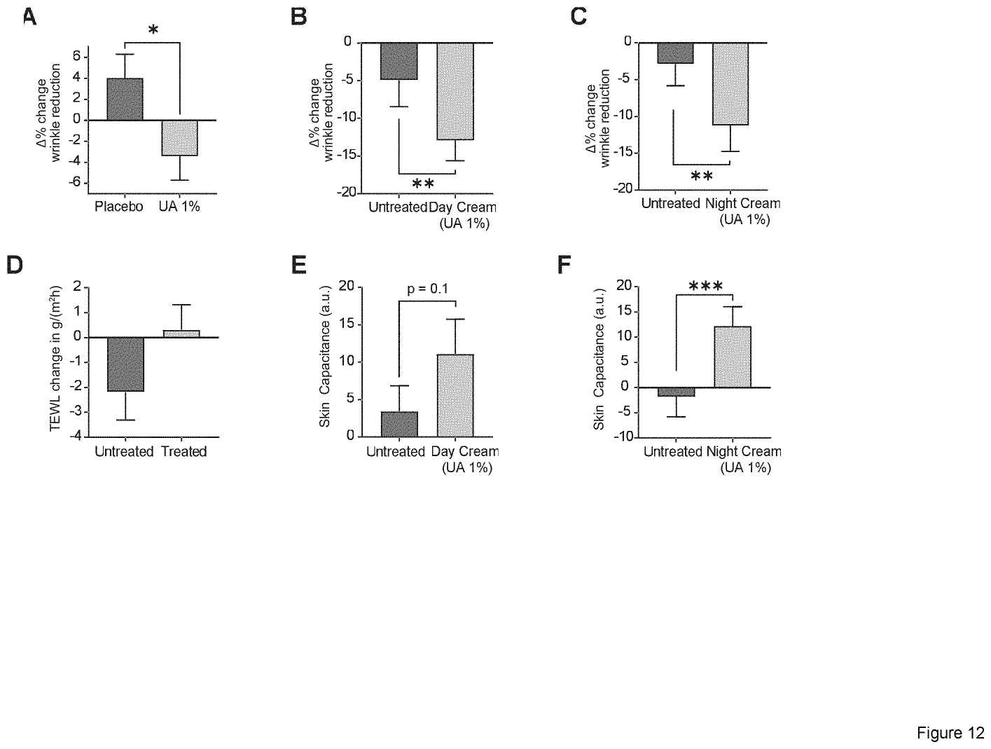

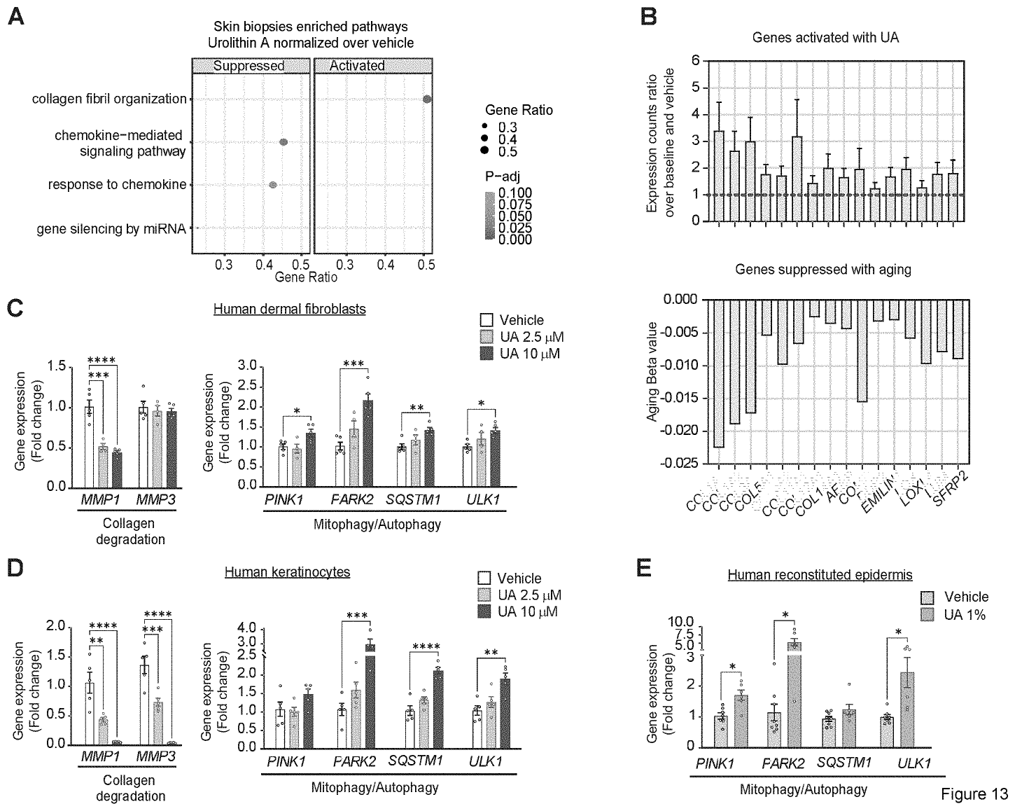

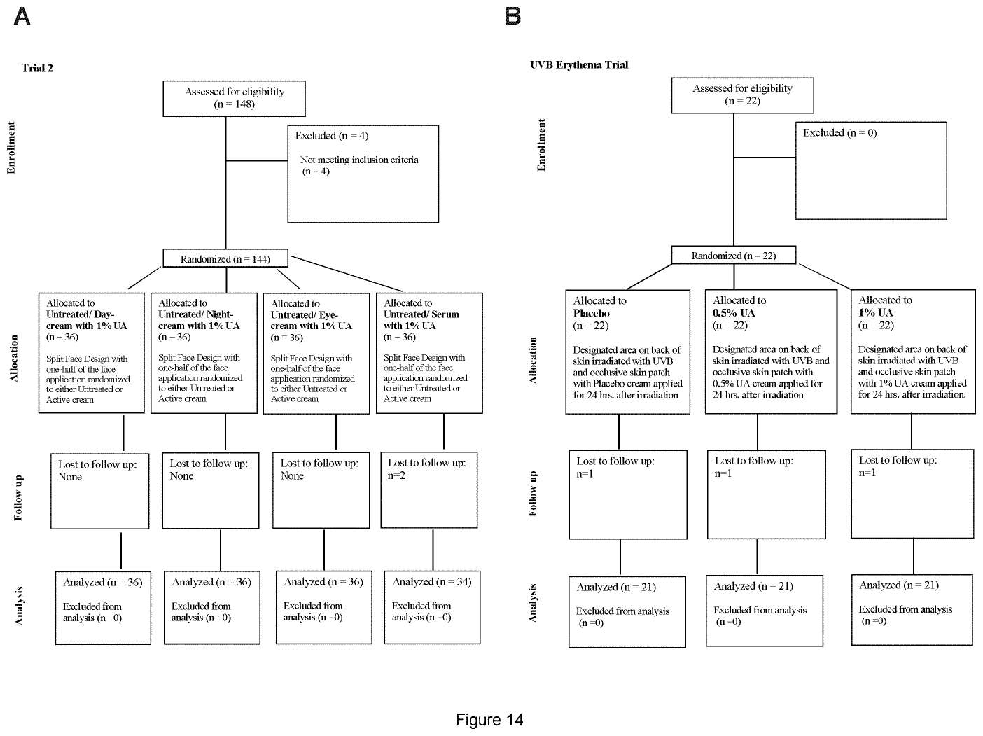

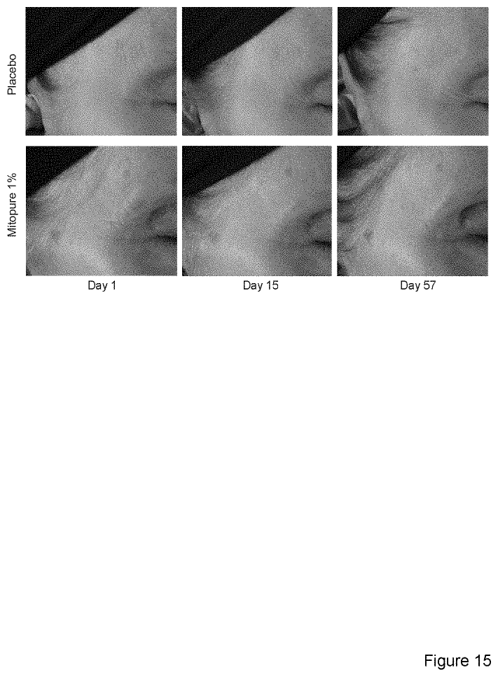

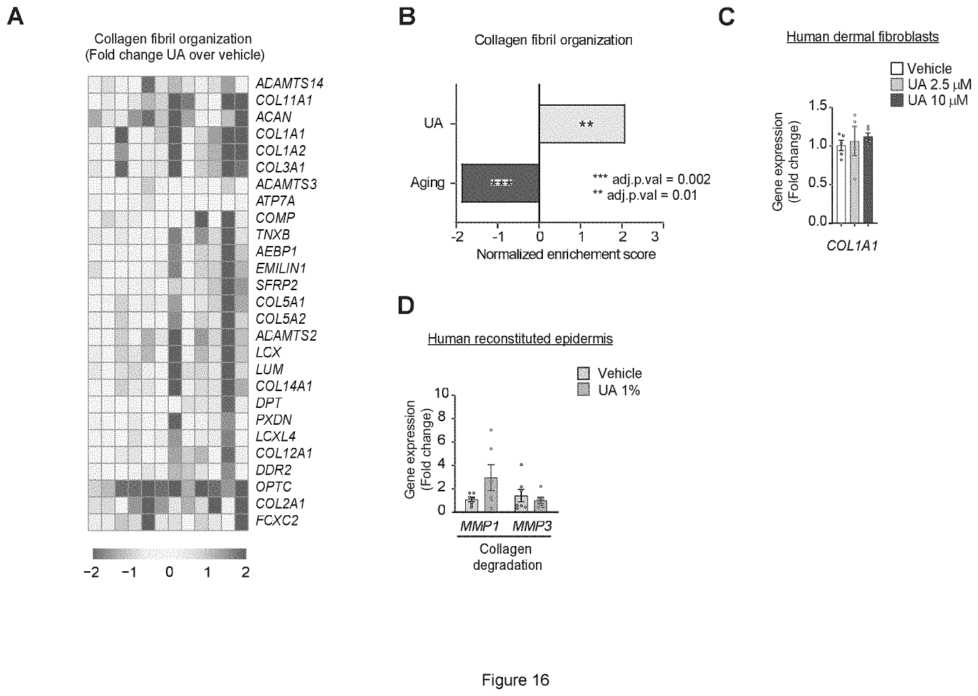

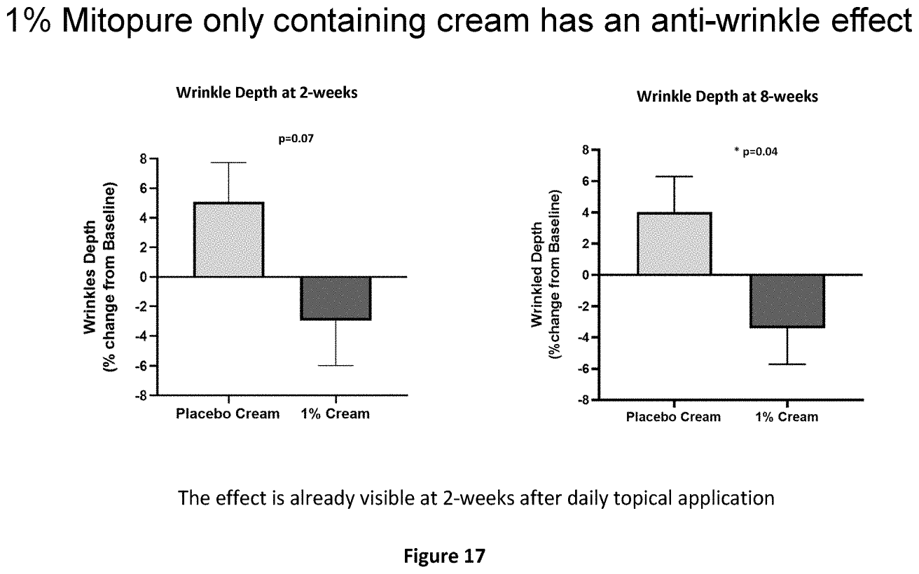

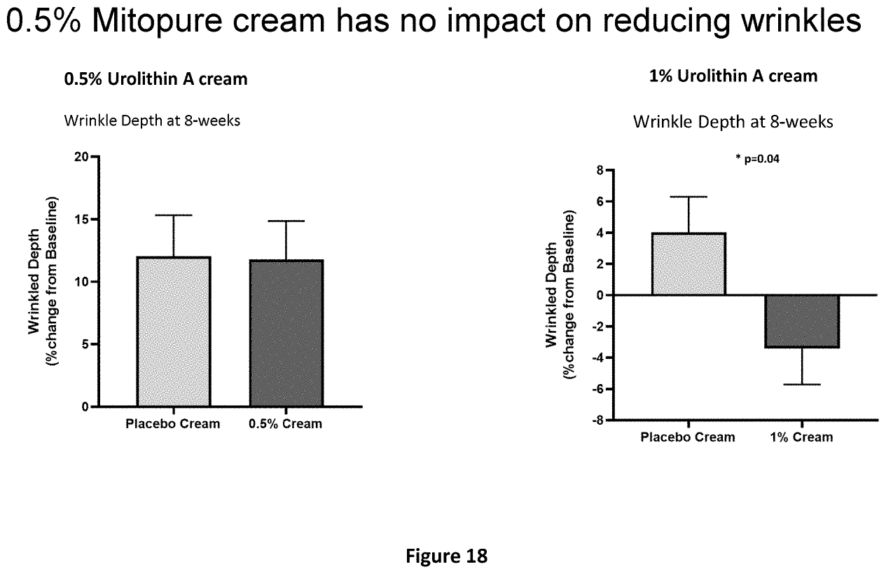

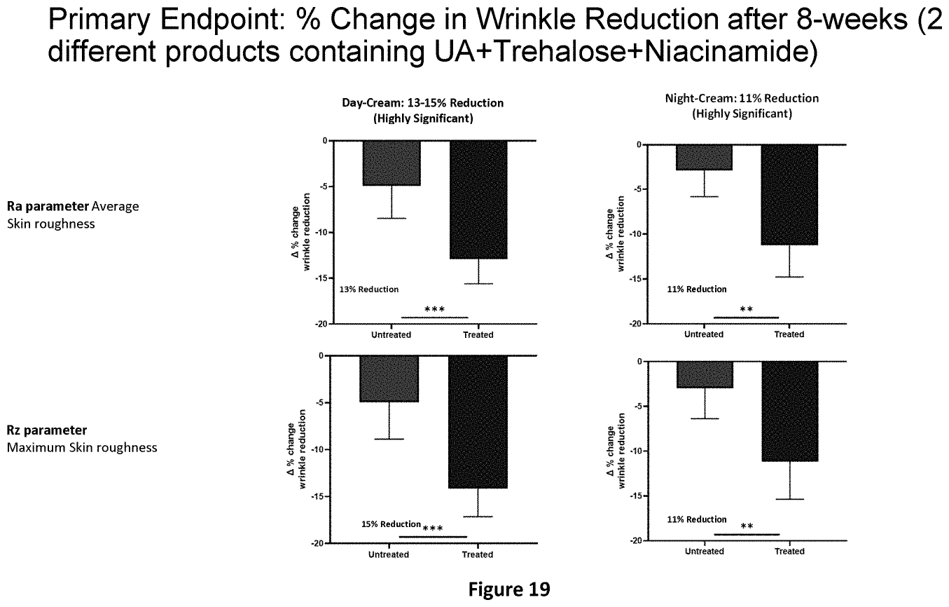

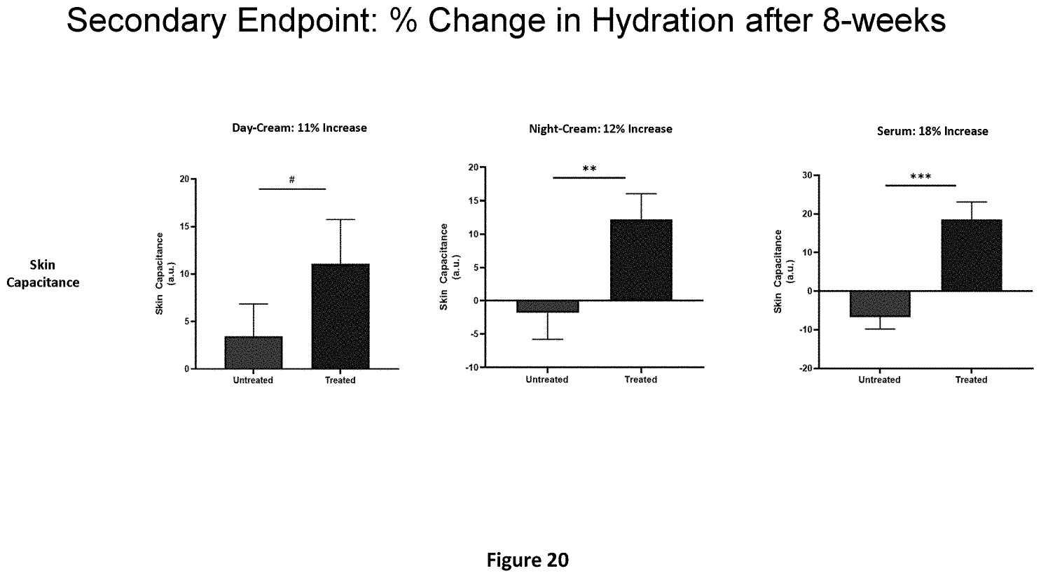

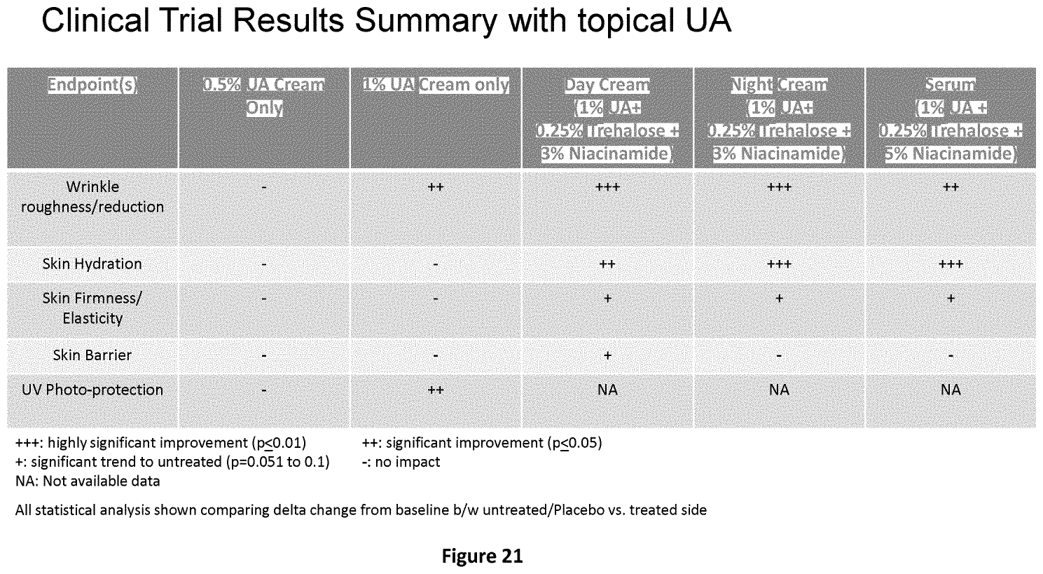

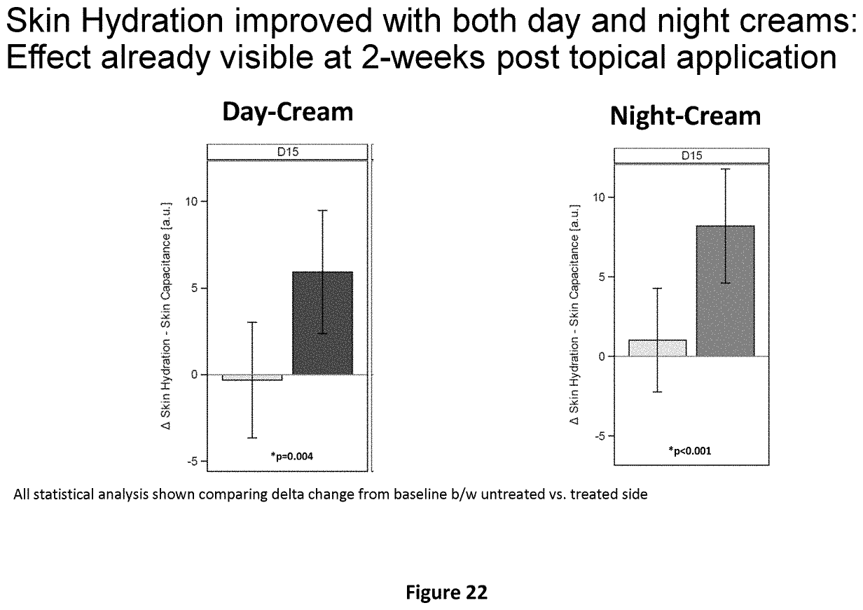

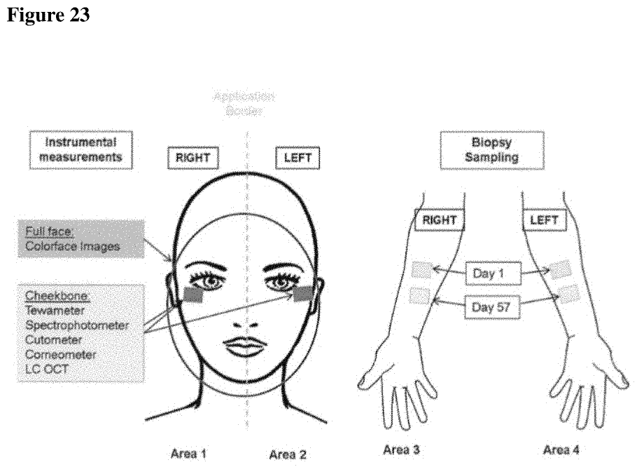

In this trial, the skincream containing Urolithin A at the higher dose of 1% showed a significant impact on reducing wrinkle depth (see FIG. 7) after 8-weeks of topical application. This anti-wrinkle efficacy was not noticed at lower doses of Urolithin A as with the 0.5% skin cream (FIG. 12) that showed no impact on the same parameter. The effect was time-dependent as effects could be observed as early as 2 weeks of topical application that were significantly enhanced at week 8 (FIGS. 13, 14). The anti-wrinkle effects with the 1% Urolithin A dose were supported by the molecular signature observed on skin biopsies that suggested a significant impact on collagen organization in aged skin with Urolithin A at 1% dose showing a significant up-regulation of collagen genes (FIGS. 2 and 4). Collagen degradation and reduced collagen synthesis with aging is a key hallmark of skin aging (FIGS. 2 and 3). The observation that a precise topical dose of 1% Urolithin A can have a wrinkle-reducing effect driven by its impact on collagen organization is the unique finding from this trial.

### Example 6 (Aging Study 2)

Clinical Trial: Evaluation of the Anti-Aging Efficacy of a Novel Cosmetic Product Containing Urolithin A, Trehalose and Niacinamide

Objective

The purpose of this exploratory study is to assess the anti-wrinkle efficacy of cosmetic products compared to an untreated control.

For this study female and male subjects, in the age of 40 to 65 years, with healthy skin and visible wrinkles in the periorbital regions will be enrolled.

After a wash-out phase the test products will be used on the half-face for the duration of 8 weeks. Assessments will be performed before, after 2, 4 and 8 weeks of product application.

Anti-wrinkle efficacy will be assessed in the periorbital regions by investigating the three-dimensional structure of the wrinkles (DermaTOP). Skin hydration, skin elasticity effects and skin barrier function will be investigated by Corneometer, Cutometer and Tewameter measurements. On a subgroup of 12 subjects the ceramide content, including barrier lipids, in the Stratum Corneum will be performed by Raman Spectroscopy.

Furthermore, images will be taken (USR-Clip) and, afterwards, evaluated by trained graders concerning anti-wrinkle and skin radiance efficacy.

Additionally, subjects will fill in a questionnaire concerning product traits after 2, 4 and 8 weeks of product application.

The study will be reviewed by an independent institutional review board (IRB) for ethical approval.

Efficacy Assessment

The following efficacy assessment(s) will be performed:
Skin roughness—Ra by DermaTOP [μm]
Skin roughness—Rz by DermaTOP [μm]
Skin Firmness—R0 (Uf) by Cutometer [mm]
Skin Elasticity—R7 (Ur/Uf) by Cutometer
Skin hydration—Skin capacitance by Corneometer [a.u.]
Transepidermal Water Loss (TEWL) by Tewameter [g/(m²h)]
Ceramides by Raman Spectroscopy [a.u.] on a subgroup of n=12 subjects per test product group
Photo documentation by USR-Clip (Hasselblad)
Image grading for skin radiance and wrinkle improvement [Trained graders]
Questionnaire of product traits

Type of Product(s)

Face care products (1 Day cream, 1 Night cream, 1 Eye cream, 1 Serum)

Components of the (Day Cream, Night Cream, Eye Cream, Serum)

Day Cream
1% urolithin A
3% niacinamide
0.25% trehalose
0.0015% Nicotiana sylvestris leaf cell culture
1.65% saccharide isomerate

Other constituents of the day cream are: Aqua (Water), Glycerin, Butylene Glycol, Dicaprylyl Carbonate, Glyceryl Stearate Citrate, Squalane, Cetyl Alcohol, Ammonium Acryloyldimethyltaurate/Vp Copolymer, Butyrospermum Parkii (Shea) Butter, Hydroxyacetophenone, Xanthan Gum, 1,2-Hexanediol, Caprylyl Glycol, Tocopheryl Acetate, Sodium Phytate, Camellia Sinensis Leaf Extract, Hydrolyzed Opuntia Ficus-Indica Flower Extract, Alcohol, Sodium Lactate, Carbomer, Citric Acid, Sodium Citrate, Coco-Glucoside, Sodium Hyaluronate, Tocopherol, Palmitoyl Tripeptide-1, Palmitoyl Tetrapeptide-7.

Night Cream
1% urolithin A
3% niacinamide
0.25% trehalose
0.5% caffeine
0.5% D-panthenol
2.75% saccharide isomerate
0.0875% hydrolyzed Opuntia ficus-indica flower extract
0.002% Opuntia ficus-indica stem extract
0.0002% Opuntia ficus-indica callus culture extract

Other components of the night cream are: aqua (water), glycerin, butylene glycol, diglycerin, dicaprylyl carbonate, saccharide isomerate, glyceryl stearate citrate, ammonium acryloyldimethyltaurate/vp copolymer, squalane, sorbitol, behenyl alcohol, cellulose, undecane, hydroxyacetophenone, tridecane, sodium stearoyl glutamate, 1,2-hexanediol, caprylyl glycol, Camellia sinensis leaf extract, alcohol, citric acid, sodium lactate, sodium citrate, carbomer, coco-glucoside, sodium hyaluronate, tocopherol, palmitoyl tripeptide-1, palmitoyl tetrapeptide-7,

Eye Cream
1% urolithin A
1% niacinamide
0.25% trehalose

Other components of the eye cream are: aqua (water), glycerin, dicaprylyl carbonate, propanediol, squalane, undecane, caprylic/capric triglyceride, glyceryl stearate citrate, 1,2-hexanediol, behenyl alcohol, cetyl alcohol, butylene glycol, tridecane, sodium polyacrylate, pullulan, hydroxyacetophenone, ethylhexylglycerin, caprylyl glycol, xanthan gum, Camellia sinensis leaf extract, Nannochloropsis oculata extract, pentylene glycol, sodium hydroxide, Tropaeolum majus flower/leaf/stem extract, alcohol, sodium lactate, carbomer coco-glucoside, sodium hyaluronate, tocopherol citric acid, Capsicum annuum leaf extract, palmitoyl tripeptide-1, palmitoyl tetrapeptide-7.

Serum
1% urolithin A
5% niacinamide
0.25% trehalose
0.5% caffeine
0.825% saccharide isomerate
1% D-Panthenol
0.0875% hydrolyzed Opuntia ficus-indica flower extract

Other components of the serum are: aqua (water), glycerin, butylene glycol, caprylic/capric triglyceride, hydrogenated lecithin, hydroxyacetophenone, ammonium acryloyldimethyltaurate/vp copolymer, xanthan gum, 1,2-hexanediol, caprylyl glycol, sodium pca, Camellia sinensis leaf extract, alcohol, erythritol, phytic acid, sodium lactate, sodium hydroxide, Chondrus crispus (carrageenan), pentylene glycol, citric acid, carbomer, coco-glucoside, sodium hyaluronate, benzoic acid, sodium citrate, sorbic acid, palmitoyl tripeptide-1, palmitoyl tetrapeptide-7, Nicotiana benthamiana hexapeptide-40 sh-polypeptide-47

Study Design
Exploratory
Randomized
Blinding of sponsor label (de-branded product)
Intra-individual comparison

Type of Statistics
Comparison to untreated for each group/subgroup (n=12) for Raman measurements
Comparison between assessment times for each group/subgroup (n=12) for Raman measurements

Assessment Times
Baseline (day 1)
Day 15 (after 2 weeks of application)
Day 29 (after 4 weeks of application
Day 57 (after 8 weeks of application)

TEST MATERIALS

| Study Group | Code/proderm | Product/Code/Sponsor/Concentration |
|---|---|---|
| Group 1 | A | Untreated |
| | B | Day cream |
| Group 2 | A | Untreated |
| | C | Night cream |
| Group 3 | A | Untreated |
| | D | Eye cream |
| Group 4 | A | Untreated |
| | E | Serum |

The test material(s) will be cosmetic products, as furnished by the sponsor. Test and control materials, if applicable, will be identified by a proderm code (e.g., “A”, “B” etc.) and/or by a sponsor identification code in a separate delivery form. Test materials will be used undiluted or diluted, as specified in the delivery form. The sponsor will identify potential hazard of the test materials furnished by the sponsor or his designee(s) associated with this study. It will be the responsibility of the Sponsor to determine, for each batch of product, the identity, strength, purity, composition and other characteristics that appropriately define the test substances before their use in the study. The determination of the stability of the test substances and documentation of methods of synthesis or derivation are also the sponsor's responsibility. The test materials will be stored at room temperature in the containers in which they are received unless otherwise specified in the delivery form. Test material remaining at the conclusion of the study will be destroyed at least 6 weeks after issuance of the final report unless requested otherwise.

Test Area
Face (split-face)
Measurement area:
Crow's feet region: DermaTOP
Cheek bone: Cutometer, Corneometer, Tewameter, Raman

Assignment of Test Areas

Randomization:

Subjects will be divided equally in 4 groups. Group 1 will receive treatments A and B, group 2 A and C, group 3 A and D and group 4 A and E. Treatments will be randomly and balanced assigned to left and right test area in each group.

Wash-Out Product

ISANA MED, Wash lotion for sensitive skin

Length of Wash-Out Phase

At least 7 days.

Standard Washing Procedure

The subjects will receive a wash-out product (silicon-free, soap/alkali-free, paraben-free) provided by the study site. The wash-out product will be used 7 days before the study start and throughout the study according to the following instructions:

The wash-out product will be used once daily in the evening and only water in the morning during the wash-out phase and throughout the course of the study (directly before test product application).

Application Volume

Approximately 2 mg/cm²

According to normal use conditions approximately a hazelnut-sized for day cream, night cream, serum to half of the face and a pea-sized for eye cream amounts the area around the eyes. The correct amount of test products to be applied will be demonstrated by a technician at the study site.

Application Mode and Frequency

The test product will be applied once or twice daily by the subjects at home. If the test product is used by the subjects themselves appropriate instructions will be given to the subjects orally and writing.

The following application frequency for the individual test products for 8 weeks will be performed:
Code B: Day cream: once daily in the morning
Code C: Night cream: once daily in the evening
Code D: Eye cream: twice daily in the morning and in the evening (gently pat around the eye area)
Code E: Serum: twice daily in the morning and in the evening

The first application of the test product will take place at the study site under the supervision of a technician on day 1, one area will stay untreated. The test material will then be applied 8 weeks by the subjects at home, according to application training.

On day 15, 29 and 57, subjects will be instructed not to apply the test products in the morning before visiting the study center, the morning application will take place at the site after all assessments.

Length of Wash-Out Phase

At least 7 days.

Standard Washing Procedure

The subjects will receive a wash-out product (silicon-free, soap/alkali-free, paraben-free) provided by the study site. The wash-out product will be used 7 days before the study start and throughout the study according to the following instructions:

The wash-out product will be used once daily in the evening and only water in the morning during the wash-out phase and throughout the course of the study (directly before test product application).

Wash-Out Product

ISANA MED, Wash lotion for sensitive skin

Study Population

A minimum of 144 subjects will be recruited for this test from the study site's location and the neighboring communities so that about 120 subjects are expected to finish in the study. Subjects who dropout after randomization into the study will not be replaced. All subjects will have a complete understanding of the test procedure.

All below mentioned inclusion, exclusion criteria and instructions for the subjects will be checked by a questionnaire before the start of the study and during the study. Conditions developing during the course of the study listed in the exclusion criteria and instructions as well as protocol deviations do not necessarily lead to the subject's exclusion. The investigator decides whether the subject is still eligible.

Inclusion Criteria
Written Informed Consent to participate in the study
Willingness to actively participate in the study and to come to the scheduled visits
Female and male
From 40 to 65 years of age
BMI<30 kg/m2
Healthy skin in the test areas
Uniform skin color and no erythema or dark pigmentation (except for subjects with ethnicity Black) in the test area
Visible wrinkle in the face (grade 3 to 6 according to proderm scale) see Appendix 2
Ethnicity: at least one Black, Asian and Hispanic subject per study group

Exclusion Criteria
Female subjects: Pregnancy or lactation
Drug addicts, alcoholics
AIDS, HIV-positive or infectious hepatitis
Conditions which exclude a participation or might influence the test reaction/evaluation
Participation or being in the waiting period after participation in cosmetic and/or pharmaceutical studies pertaining to the test area
Active skin disease at the test area
Documented allergies to face/eye care products
Diabetes mellitus
Cancer not being diagnosed as cured and requiring chemotherapy, irradiation and/or hormonal treatment within the last 2 years
One of the following illnesses with reduced physical capability/fitness: asthma (symptom-free allergic asthma is not an exclusion criterion), hypertension (if not adjusted with medication), cardiovascular diseases
Epilepsy
Wounds, moles, tattoos, scars, irritated skin, excessive hair growth, freckles, etc. at the test area that could influence the investigation
Sun-tanned skin (important for the subgroup of subjects planned for Raman measurements)
Regular use of tanning beds
Any topical medication at the test area within the last 7 days prior to the start of the study
Systemic therapy with immuno-suppressive drugs (e.g., corticosteroids) and/or antihistamines (e.g., antiallergics) and/or within the last 7 days prior to the start of the study
Past cosmetic surgery procedure in the test area (e.g., laser, facelift)
Cosmetic surgery procedure in the test area, e.g., IPL (Intensed Pulsed Light), botox, chemical peel, dermabrasion within the last 2 years prior to the start of the study and/or throughout the entire course of the study
Medical treatment for wrinkle reduction (e.g., peeling with vitamin A or fruit acids) on the face within the last 2 weeks prior to the start of the study

Instructions for Subjects

Instructions prior to the start of the study:

The subjects will be instructed not to . . .
Not applicable

The subjects will be instructed to . . .
Not applicable

Instructions throughout the course of the study:

The subjects will be instructed not to . . .
apply any detergents (e.g., soaps, shampoos, bath and shower products) other than the provided wash-out product to the test area throughout the entire wash-out phase
apply any detergents (e.g., soaps, shampoos, bath and shower products) to the test area in the morning prior to the scheduled visit
apply any leave on cosmetics (e.g., creams, lotions) other than the test product to the test area throughout the entire course of the study
apply any decorative cosmetics (e.g., mascara, eye shadow, make-up, powder and other cover products) to the test area in the morning prior to the scheduled visits
bring the test area in contact with water (e.g., no bathing and swimming, only careful showering excluding the test area) within the last 2 h prior to the instrumental measurements
visit the sauna or get a massage in the test area throughout the entire course of the study
excessive expose the test area to the sun, UV-therapy and/or artificial tanning throughout the entire course of the study
apply any cover cream, tinted day cream and face powder with moisturizing and anti-aging efficacy to the test area throughout the entire course of the study
drink any caffeinated beverages (e.g., coffee, cola, black tea) within the last 2 h prior to the instrumental measurements
smoke within the last 2 h prior to the instrumental measurements

The subjects will be instructed to . . .
use the detergent (wash-out product) provided by proderm in the test area starting from day −7 until the end of the study according to the given instructions
apply the test product(s) to the test area on the according side of with the following frequency (according to proderm's instructions): once daily for group 1 and 2 and twice daily for group 3 and 4 throughout the entire course of the study
continue to use the normally used decorative cosmetics (e.g., mascara, eye shadow, powder) in the test area, but not to change the brand or use new products throughout the entire course of the study
perform the last application of the test product(s) on the previous day prior to the scheduled visits

Informed Consent

For studies with subjects the following procedure will be effective: Each subject must provide the investigator/investigator's designee with written informed consent prior to enrollment in the study. On request they will receive a copy of the informed consent statement.

The original signed copy for each subject participating in the study will be retained in the investigator's study records. The consent statement shall meet the requirements of any applicable regulation. The investigator or the investigator's designee will inform each subject as to the purpose and nature of the study in compliance with applicable regulations.

The subjects are informed that they can withdraw their consent at any time and that they can stop their participation in this study at any time, without disadvantages. They are also informed that proderm can also prematurely terminate their participation in the study, for example for administrative reasons.

In case of image taking for marketing purposes the subjects will be asked if they agree that images are captured that may be used by the sponsor for marketing and promotion purposes. Each subject that agrees will sign the related specific informed consent form. Only the images of subjects who give their informed consent may be used for marketing and promotion purposes.

Test Procedure

Day −7 (wash-out):

The subjects will come to the study site. They will be informed about the study and give their written consent. Subjects will be provided with the wash-out product. They will be instructed to use only the wash-out product in the test area until their next visit at the study site. −Day −7 to −1:

The subjects will apply the wash-out product according to instructions.

Day 1:

The subjects will return to the study site, standardized images (USR-CliP) of the face will be taken. After acclimatization for at least 30 minutes, instrumental measurements (baseline) on the test areas will be performed. Raman measurements will be performed on a subpanel of 12 randomly chosen subjects. The test product(s) will be issued to the subjects with instructions to use them on the assigned test areas. The assignment of test product(s) will be done according to a randomization scheme provided by proderm. The first product application will be performed under the guidance of a technician. Further application(s) will be performed by the subjects at home.

Day 2 to day 14:

Subjects will use the test product(s) as described above (see ‘application mode’). The last product application will be performed on day 14.

Day 15:

Subjects will return to the study site. A standardized image (USR-CliP) of the face will be taken. After acclimatization instrumental measurements on the test areas will be repeated. Additionally, the subjects will fill in a questionnaire concerning test material traits of the test product.

Afterwards product application will be performed under the guidance of a technician. If applicable, one further application will be performed by the subjects at home.

Day 16 to day 28:

Subjects will use the test product(s) as described above (see ‘application mode’). The last product application will be performed on day 28.

Day 29:

Subjects will return to the study site. A standardized image (USR-CliP) of the face will be taken. After acclimatization instrumental measurements on the test areas will be repeated. Additionally, the subjects will fill in a questionnaire concerning test material traits of the test product.

Afterwards product application will be performed under the guidance of a technician. If applicable one further application will be performed by the subjects at home.

Day 29 to day 56:

Subjects will use the test product(s) as described above (see ‘application mode’). The last product application will be performed on day 56.

Day 57:

Subjects will return to the study site. A standardized image (USR-CliP) of the face will be taken. After acclimatization instrumental measurements on the test areas will be repeated. Additionally, the subjects will fill in a questionnaire concerning test material traits of the test product.

The remaining test materials will be returned.

A deviation of ±2 days during the application phase will be accepted, since no substantial influence on the outcome of the study is expected.

After the end of the study, 3 trained graders will additionally evaluate the images regarding anti-wrinkle efficacy. The grader(s) will rank each pair of images (before and after 2, 4 and 8 weeks product application, respectively) from all subjects regarding their wrinkle improvement in a blinded and randomized manner.

Climatic Conditions

The instrumental measurement(s) will take place in an air-conditioned room at a temperature of 22±2° C. and at 50±7.5% relative humidity. Before measurements the subjects will stay in the climatized room for at least 30 minutes.

Test Schedule

A scheme of the test procedure is given as appendix 1 to protocol.

Instrumental Measurement(s)

The following instrumental measurement(s) will be performed:

DERMATOP blue (Eo Tech SA, Marcoussis France): Using phase-shifted and gray-coded measurements of human skin the three-dimensional surface structure of the investigated skin site is captured. The measuring principle is based on digital fringe projection. The fringes that are projected under a defined triangulation angle onto the surface of the measured target with a sinus-like intensity of brightness are detected with a CCD camera. The three-dimensional skin surface profile is calculated from the position of the fringes in combination with the gray values of each pixel. From the captured three-dimensional structure, roughness parameters are calculated. Parameters: Rz and Ra are chosen, representing mainly the rough structure (Rz) or the finer skin structure (Ra). A decrease in the roughness parameters Rz and Ra corresponds to a decrease in the degree of skin roughness.

−1 measurement per test area and assessment time

CUTOMETER MPA 580 (Courage & Khazaka, Cologne, Germany). The measuring principle is based on suction of the skin into an aperture of 2 mm in diameter. In the measuring head, a defined vacuum of 300 mbar is induced. The measurement head is placed on the skin with a defined pressure and the skin is sucked into the opening of the measuring head for 5 seconds with a subsequent measuring period of another 5 seconds after release. Using an optical measuring system, it is detected contactless how far the skin is sucked into the measuring head; this value and the skin's ability to return to its original state gives a measure for the elasticity of the skin.

Parameters:

R0 Total elasticity (=Uf=first max. amplitude, highest point of the first curve,)

R7 Quotient of elastic relaxation to total elasticity (=Ur/Uf=portion of the elasticity compared to the complete curve, the closer the value is to 1 (100%) the more elastic the curve

−1 measurement per test area and assessment time

CORNEOMETER CM 825 (Courage & Khazaka, Cologne, Germany): The measurement of stratum corneum hydration will be performed by the electrical capacitance method with the corneometer. The measuring principle is based on changes in the capacitance of the measuring head, functioning as a capacitor. Between the conductors consisting of gold, an electrical field is built. By these means, the dielectricity of the upper skin layer is measured. Because the dielectricity varies as a function of the skin's water content, the stratum corneum hydration can be measured.

−5 measurements per test area and assessment time

TEWAMETER® TM 300 (Courage & Khazaka, Cologne, Germany): Transepidermal water loss (TEWL) is a non-invasive method to measure the barrier function of the skin and is regarded as a sensitive parameter to quantify skin barrier damage. Briefly, the insensible water evaporation from the skin is measured by placing cylindric open chamber with two hygrosensors at defined distance to the skin.

The probe is held in place for each measurement for 30 seconds. This is to assure that a stable value has been established. The first part of the measurements belongs to this equilibration phase. The values of the last 10 seconds (=10 values) are averaged as the actual measurement value.

−1 measurement per test area and assessment time

USR-CLIP (proderm, Schenefeld, Germany): The photo documentation will be performed using a high resolution digital camera unit for standardized and reproducible clinical photography (USR-CliP) with Hasselblad camera H5D-50c with high resolution 50 megapixels.

Photography will use calibrated colors so that further assessments can be performed at a later phase.

−1 image(s) per test area and assessment time

The pseudonymized images can be used for marketing purposes. A corresponding informed consent will be signed by the subjects.

RAMAN SPECTROMETER Model gen2-SCA Skin Analyzer (River Diagnostics, Rotterdam, Netherlands): Raman spectra are obtained by focusing low power laser light in the skin and by measuring the Raman scattered light from the laser focus. A small part of the scattered light is found at wavelengths higher than the incident laser light. This part of the scattered light provides information about the molecular composition of the skin. Raman spectra are combined, to present concentration profiles of molecular species.

Fingerprint profiles for NMF detection in SC:

The fingerprint profiles are calculated from Raman spectra (wave number range from 400 to 1800 cm-1) that will be taken at different depths. The amount might vary according to differences in skin depth and measurement conditions.

For NMF detection profiles will be defined at skin depths of 0, 4, 8, 12+/−4 μm measured from the surface of the skin, in steps of 4 μm with an integration time of 5 seconds, one frame per depth;
approximately 6 profiles per test area and assessment time
50 μm pinhole

The investigator will manually control all fingerprint profiles. Invalid curves (e.g., clearly inconsistent in keratin curve pattern) will be excluded.

The following parameters will be assessed from obtained fingerprint profiles by the Raman software (Skin Tools 3, RiverD):
Ceramides [a.u.]

Investigational Method(s)

The following investigational methods will be performed:

Questionnaire for Product Acceptance by Subjects

Product traits will be assessed by the subjects and will consist of closed questions with predefined identical options to tick.
−2=Fully disagree
−1=Rather disagree
1=Rather agree
2=Fully agree

A scheme of the questionnaires is given as Appendix 3 to protocol

Image Evaluation

Subsequent Ranking of Images by Trained Graders:

A visual evaluation of the images taken as described above. A computerized system randomizes the images of one subject, enabling blind visual ranking of the images. Images are rated on color-calibrated monitors. The NEC SpectraView Reference 271 is a professional wide gamut 10-bit P-IPS LCD monitor for color critical applications with high picture quality and color accuracy. 3 trained graders will rank each pair of images regarding to the parameter “skin radiance” (taking into account of complexion, transparency, brightness, luminosity) and “anti-wrinkle efficacy” (at the area of the crow feet's) in a blinded and randomized manner.

For the evaluation of code D, the eye cream, the images will be cropped to show only the eye area where only this test product has been applied and only there the assessment will be done. Per subject and test area and assessment time, images before (baseline) and after product application (day 15, day 29 and day 57) will be presented simultaneously and for each post-treatment separately to the graders on a visual analogue scale of:

−50=left image better to +50=right image better. It is not allowed to rank both images equal. After the end of the study values will be de-randomized and decoded to

−50=baseline better to +50=post treatment (day 15, day 29, day 57) better

In this trial, utilizing topical products containing a mix of Urolithin A at 1%, trehalose (0.25%) and niacinamide (1-5%) an even better wrinkle reduction profile was observed (13-15% reduction in wrinkles) compared to a cream containing Urolithin A at 1% alone (4-6% reduction)—See FIG. 7. In addition the skincare products when applied topically for 8-weeks also enhanced the skin hydration profile (FIG. 8). The significant effect on wrinkle reduction and improved hydration was maintained across a range of products containing the three ingredients of urolithin A, trehalose and niacinamide as shown in FIG. 7. The effects of the combination of these actives was independent of the model of application and timing of the day of application that also different in as the day cream was applied once every morning and the night-cream was applied once in the evening before sleeping.

Results from Aging Study 1 (Example 5) and Aging Study 2 (Example 6)

Demographics

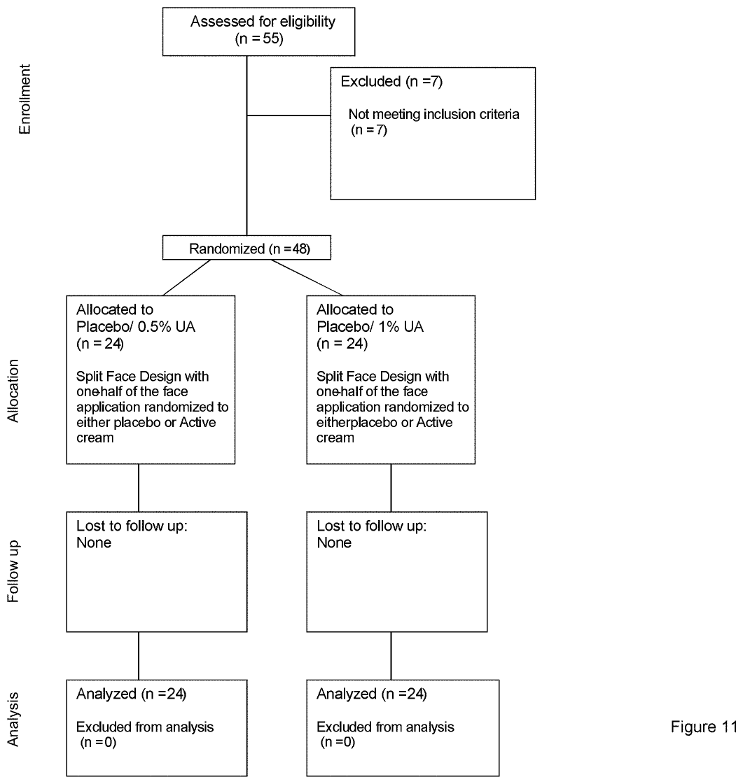

55 Participants were screened in the placebo-controlled, first aging study. Of them, n=48 middle aged women subject with mean age of 58.6±6.6 years were randomized (FIG. 11) to two study arms with a split-face/arm study design with a lower dose active group compared to placebo and a second higher dose active group compared to placebo. All participants completed the trial and there were no dropouts. Ethnicity of all participants was Caucasian. In the second aging study, n=144 middle-aged subjects with mean age of 55.4±6.6 years were randomized using a split face study design with a treatment side compared to an untreated side. n=142 subjects completed the study protocol with two drop-out subjects in this trial (FIG. 14). There were 35 males (25%) and 107 females (75%) in the final analysis of which n=2 (1%) were Black, 125 (88%) were Caucasian, n=4 (3%) East Asian, n=3 (2%) Hispanic and n=8 (6%) West Asian ethnicity. In the UVB-mediated erythema trial (Example 4)(placebo-controlled, Photo-damage study), n=22 subjects (10 male (48%), 11 female (52%) with mean age 44.0±14.1 years and Caucasian ethnicity) were enrolled. There was one drop-out subject and n=21 subjects completed the study protocol (Supplementary FIG. 14B).

In all cases, the dosage of the effected test products remained unchanged, and all subjects recovered without sequelae. In the UVB erythema trial, two adverse reactions occurred in terms of skin irritation with erythema in one subject. The reactions were of mild severity and resolved without sequelae.

Wrinkle Reduction with 1% Topical UA Application

In the aging study 1, wrinkle reduction was measured after 2, and 8-weeks of test treatment application (FIG. 15A). The UA 1% treatment group showed a significant reduction in wrinkle depth at 8-weeks (p=0.04; FIG. 12A) compared to the placebo treated side. UA at 0.5% did not showed any significant effect compared to the Placebo. In the second aging study, UA at 1% was found to have a significant impact on reducing wrinkles (finer and rougher skin structures computed as Ra and Rz, respectively) with the effect already manifesting after 2 weeks of application and maintained with 4- and 8-weeks of application, compared to the untreated side (Table 5).

TABLE 5
Measurements by DermaTOP of skin roughness (wrinkle) - Mean values, standard
deviations, treatment comparison on differences to baseline, and time
comparison on raw data for day-cream containing 1% Urolithin A.

| | | | | | | p-Values | | | |
|---|---|---|---|---|---|---|---|---|---|
| | | | | Mean Values ± SD | | | | | Treat. |
| | | | | Raw | Diff. | Time Comp. | | | Comp. |
| Parameter | Time | Code | n | Data | to BL | vs. BL | vs. D 15 | vs. D 29 | vs. A |
| Skin | BL | A | 33 | 27.41 ± | — | — | — | — | — |
| Roughness - | | | | 7.14 | | | | | |
| Ra by | | B | 34 | 28.36 ± | — | — | — | — | — |
| DermaTOP | | | | 8.05 | | | | | |
| [μm] | D 15 | A | 34 | 27.22 ± | −0.44 ± | 0.562 | — | — | — |
| | | | | 8.13 | 4.25 | | | | |
| | | B | 34 | 24.87 ± | −3.33 ± | <0.001 | — | — | <0.001 |
| | | | | 7.33 | 3.47 | | | | |
| | D 57 | A | 35 | 27.41 ± | −1.29 ± | 0.203 | 0.667 | 0.424 | — |
| | | | | 10.32 | 5.72 | | | | |
| | | B | 35 | 24.65 ± | −3.81 ± | <0.001 | 0.405 | 0.185 | 0.008 |
| | | | | 7.61 | 4.79 | | | | |
| Skin | BL | A | 33 | 111.41 ± | — | — | — | — | — |
| Roughness - | | | | 34.99 | | | | | |
| Rz by | | B | 34 | 114.43 ± | — | — | — | — | — |
| DermaTOP | | | | 33.63 | | | | | |
| [μm] | D 15 | A | 34 | 111.01 ± | −1.49 ± | 0.658 | — | — | — |
| | | | | 37.95 | 18.87 | | | | |
| | | B | 34 | 99.62 ± | −13.79 ± | <0.001 | — | — | <0.001 |
| | | | | 29.05 | 17.49 | | | | |
| | D 57 | A | 35 | 111.89 ± | −5.85 ± | 0.211 | 0.655 | 0.413 | — |
| | | | | 46.78 | 26.37 | | | | |
| | | B | 35 | 97.70 ± | −17.08 ± | <0.001 | 0.268 | 0.110 | 0.009 |
| | | | | 31.16 | 20.99 | | | | |

bold p-Value: significant (p ≤ 0.05)
A: Untreated side of the face;
B: 1% UA Treated side of the face
Ra: average skin roughness;
Rz: maximum skin roughness
BL: Baseline;
D 15: Day 2-weeks of application;
D 57: 8 weeks of application

The effect was independent of timing and matrix of the product applied as both a day and night cream application with 1% UA resulted in similar wrinkle reducing effects (FIG. 12B, C).

Impact on Skin Barrier and Skin Hydration in the Aging Studies

Daily application of the creams (day or night creams) containing 1% UA significantly improved skin hydration after 2 weeks of use in comparison to pre-test treatment level (Supplementary table 3 to add). The observed improved skin hydration levels were maintained also after 8 weeks of application of a night cream in comparison to the untreated side (FIG. 12E, F).

Supportive Biomarkers

We investigated biological pathways impacted by topical UA application performing RNA-seq transcriptomics of forearm skin biopsies from the aging study I. Gene set enrichment analysis (GSEA) was applied to identify significant pathways changed by UA comparing end of the study to baseline, and normalizing results over vehicle. “Collagen fibril organization” was the most significantly activated pathway by UA (FIG. 13A and FIG. 16A), with UA application also reducing gene sets related to chemokine response (FIG. 13A). We further studied which UA regulated pathways are involved in the natural skin aging process. Analysing a publicly available skin aging clinical study 12, the “Collagen fibril organization” was a common signature, both induced by UA and downregulated with skin aging (FIG. 16B). This gene set includes collagen genes encoding for fibrillar type I and III collagen (FIG. 13B), known to decline in skin with chronological aging 13, photo-aging and smoking 14. UA also rescued age-associated decline of genes involved in skin homeostasis. Among them, EMILIN-1, regulating 3D structure and elasticity 15 and AEBP, encoding for a collagen-interacting protein required for optimal tissue repair 16 (FIG. 13B). These data are in line with the positive impact of UA on wrinkle reduction, a process that is driven by impaired collagen production and organization in the skin.

The GSEA results suggest an impact of UA on the dermis, the skin layer producing and secreting collagen. To better understand mechanisms of action of UA in dermal cells, we treated primary human dermal fibroblasts with UA at either 2.5 μM or 10 μM, or with vehicle, for 24 hours. Surprisingly, this short-term exposure to UA did not alter collagen gene expression (FIG. 16C). However, it suppressed the expression of the gene involved in collagen degradation, Matrix Metalloproteinases (MMP1) (FIG. 13C). Moreover, UA induced a dose-dependent increase in the expression of the mitophagy genes PARKIN and PINK1 and the autophagy genes MAP1LC3 and ULK1, compared to vehicle-treated cells (FIG. 13C).

Human primary keratinocytes treated with UA or vehicle for 24 hours showed a similar gene expression signature as dermal cells. UA led to a drastic downregulation of both MMP1 and the other key collagen degrading enzyme, MMP3 (FIG. 13D). And it upregulated autophagy and mitophagy gene expression, as in dermal fibroblasts (FIG. 13D). Finally, we employed a model of 3D reconstituted human epidermis (RHE) applied daily with the UA 1% moisturizing cream for 3 days. No change was observed in MMP1 and MMP3 gene expression (FIG. 16D). However, consistently with the data in primary cells, results confirm an early activation of mitophagy and autophagy genes in RHE applied with UA compared to Vehicle (FIG. 13E).

### Example 7

Product Acceptance Questionnaire

As set out in the protocol in Example 6, participants in the trial completed a product acceptance questionnaire. In this questionnaire a value of above 60% is considered a significant improvement. Table 6 summarises results from the questionnaire and it can be seen that, in all parameters listed, a significant improvement was seen at 8 weeks, with most listed parameters showing a significant improvement at 4 weeks.

TABLE 6

| | Week | Week | Week |
|---|---|---|---|
| | 2 | 4 | 8 |
| Day- Cream | | | |
| Skin looks revitalized and rejuventaed | 39 | 64 | 69 |
| Skin Looks full of Life and Energy | 47 | 64 | 72 |
| Skin Barrier feels strengthened and resilient | 53 | 67 | 69 |
| Skin feels hydrated, nourished and supple all day | 69 | 72 | 78 |
| Overall quality of the skin is significantly | 50 | 64 | 64 |
| improved | | | |
| Skin looks significantly healthier | 47 | 61 | 72 |
| Skin appears re-energized for optimal health | 47 | 58 | 67 |
| Night-Cream | | | |
| Skin Barrier feels strengthened and resilient | 50 | 61 | 64 |
| Skin feels hydrated, nourished and supple all day | 50 | 58 | 67 |
| Skin looks significantly healthier | 39 | 61 | 60 |
| Skin texture is smoother and softer | 58 | 67 | 64 |
| Serum | | | |
| Skin looks revitalized and rejuventaed | 53 | 62 | 68 |
| Skin Looks full of Life and Energy | 50 | 65 | 60 |
| Skin Barrier feels strengthened and resilient | 56 | 62 | 65 |
| Skin looks significantly healthier | 47 | 59 | 62 |
| Visible signs of skin aging are reduced | 38 | 50 | 62 |
| Skin feels hydrated, nourished and supple all day | 56 | 68 | 71 |

### Example 8 (3 Week User Trial)

Product Acceptance Questionnaire

A 3 week user trial was conducted in 30 healthy women, aged 40 to 65 years. Participants used the Day Cream, Night Cream and Serum as described in Example 6. Significant benefits were observed as follows:

Day cream

| | |
|---|---|
| 100% | of participants report their skin looks more |
| | radiant, brighter and glowing with health |
| 90% | say skin feels protected, stronger and |
| | revitalized |
| 86% | of participants said that skin's texture looks |
| | and feels redensified and with more volume |

Night cream

| | |
|---|---|
| 90% | of participants report their skin appears to |
| | regenerate overnight |
| 93% | agree skin feels protected, stronger and |
| | revitalized |
| 80% | say skin wakes up re-energized with a more |
| | youthful appearance |

Serum

| | |
|---|---|
| 90% | of participants report skin feels protected, |
| | stronger and revitalized |
| 80% | say skin looks more youthful, visibly |
| | renewed |
| 80% | report skin looks more radiant, brighter and |
| | glowing with health |

1. A method:
a) for improving or maintaining skin barrier function;
a) for limiting transepidermal water loss;
b) for improving skin elasticity and firmness;
c) for increasing or maintaining skin layer thickness;
d) for increasing or maintaining skin hydration;
e) for improving anti-wrinkle efficiency;
f) for improving collagen organization in the skin;
g) for reducing skin biological aging;
h) for improving skin health; and/or
i) improving skin microarchitecture,
comprising administering to a subject in need thereof an effective amount of a compound of formula (I): 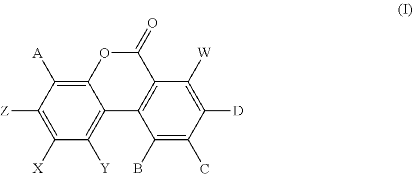
wherein:
A, B, C and D are each independently selected from H and OH;
W, X and Y are each independently selected from H and OH; and
Z is selected from H and OH.

2. A method for:
(a) maintaining or enhancing hair thickness;
(b) hair follicle cell regeneration;
(c) hair follicle cell survival;
(d) hair stem cell growth and/or regeneration;
(e) hair cell survival;
(f) hair loss prevention,
(g) hair maintenance;
(h) scalp health improvement,
(i) improving or maintaining hair strength,
(j) hair growth restoration promotion
(k) enhancing hair follicle elongation;
(l) enhancing hair matrix proliferation, for example, as measured by Ki67 levels; and/or
(m) reducing melanin clumping, for example, in pigmentary units, for example, in hair follicle pigmentary units,
comprising administering to a subject in need thereof an effective amount of a compound of formula (I): 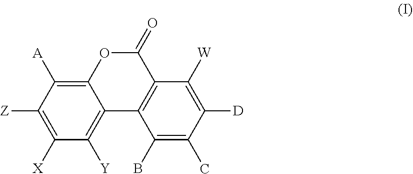
wherein:
A, B, C and D are each independently selected from H and OH;
W, X and Y are each independently selected from H and OH; and
Z is selected from H and OH.

3. A method for:
(a) renewal and/or revitalization of skin appearance;
(b) enhancement of skin energy supply;
(c) improvement in skin texture;
(d) enhancement of skin radiance;
(e) skin rejuvenation;
(f) reduction of pore and micro line visibility;
(g) improvement in skin respiration;
(h) support optimal cellular function in skin;
(i) promote/activate skin detoxifying processes;
(j) promote collagen repair, and/or
(k) improve skin stem cell health,
comprising administering to a subject in need thereof an effective amount of a compound of formula (I): 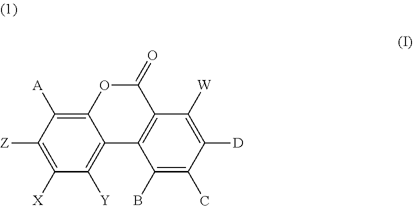
wherein:
A, B, C and D are each independently selected from H and OH;
W, X and Y are each independently selected from H and OH; and
Z is selected from H and OH.

4. The method of claim 1, wherein the compound of formula (I) is urolithin A, urolithin B, urolithin C or urolithin D, for example, urolithin A.

5. The method of claim 1, wherein the compound of formula (I), or salt thereof, is administered in the form of a composition.

6. The method of claim 5, wherein the composition is in the form of make-up, a lotion, a cream, a shampoo, a serum, a spray, a gel, a mask, a toner, a micellar water, a soap, a balm, a mousse, an oil, a sunscreen, a milk, an exfoliator, a hair treatment, or a cleanser.

7. The method of claim 5, wherein the compound of formula (I), or a salt thereof, makes up about 0.8 to about 5% w/w of the composition, for example, about 0.8 to about 2%, w/w, such as about 0.8% or about 1% w/w of the composition.

8. The method of claim 5, wherein the compound of Formula (I), or a salt thereof, has a D50 size in the range 2 to 8 μm and a D90 size in the range 6 to 20 μm.

9. A composition comprising:
a) a compound of formula (I), or salt thereof, 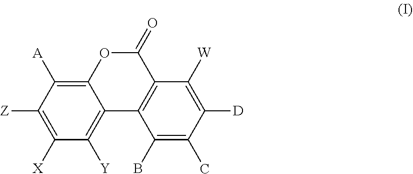
wherein:
A, B, C and D are each independently selected from H and OH;
W, X and Y are each independently selected from H and OH; and
Z is selected from H and OH;
b) trehalose; and
c) an NAD precursor, for example, niacinamide.

10. The composition of claim 9, further comprising
d) Nicotiana sylvestris leaf cell culture; and
e) saccharide isomerate; or
d) Nopa flower extract (Opuntia ficus-indica);
e) Caffeine;
f) Saccharide isomerate, and
g) a B vitamin, for example, D-Panthenol; or
d) caffeine; and
e) a B vitamin, for example, D-Panthenol; or
d) caffeine.

11. (canceled)

12. (canceled)

13. (canceled)

14. The composition of claim 9,
a) wherein the compound of formula (I) comprises 0.8% to 5% (w/w) of the composition;
b) wherein the trehalose comprises 0.05% to 1% (w/w) of the composition;
c) wherein the niacinamide comprises 0.1% to 10% (w/w) of the composition, and
d) optionally, caffeine,
wherein the caffeine if present, comprises 0.1% to 5% (w/w) of the composition.

15. The composition of claim 14, wherein:
a) the compound of formula (I) comprises 0.8% to 2% (w/w) of the composition;
b) wherein the trehalose comprises 0.1% to 0.5% (w/w) of the composition;
c) wherein the niacinamide comprises 0.5% to 5% (w/w) of the composition; and
d) wherein the caffeine, if present, comprises 0.1% to 1% (w/w) of the composition.

16. A composition, comprising:
a) a compound of formula (I), or a salt thereof, 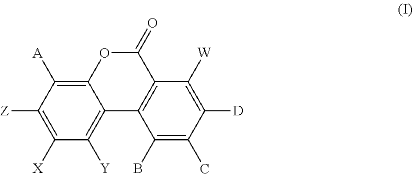
wherein:
A, B, C and D are each independently selected from H and OH;
W, X and Y are each independently selected from H and OH; and
Z is selected from H and OH;
b) an autophagy inducer; and
c) optionally a mitochondrial biogenesis promoting agent.

17. The composition of claim 16, wherein the composition comprises a mitochondrial biogenesis promoting agent.

18. The composition of claim 16, wherein the compound of formula (I) is urolithin A.

19. The composition of claim 17, wherein the autophagy inducer is selected from carbamazepine, clonidine, lithium, metformin, rapamycin (and rapalogs), rilmenidine, sodium valproate, verapamil, trifluoperazine, statins, tyrosine kinase inhibitors (for example, Akt-mTOR signaling inhibitors and beclin 1 tyrosine phosphorylation inhibitors), BH3 mimetics, caffeine, omega-3 polyunsaturated fatty acids, resveratrol, spermidine, vitamin D pterostilbene, fistein, genistein, quercetin, apigenin, kaempferol, minoxidil, actinonin, kinetin triphosphate, pifithrin-a, deferiprone, 1,10′-phenanthroline, and trehalose; and
the mitochondrial biogenesis promotor is selected from PPAR-PGC-1α axis activators (for example, bezafibrate), AMPK activators (for example, resveratrol), Sirt1 agonists (for example, quercetin, resveratrol), anti-oxidants (such as L carnitine, coenzyme Q10, MitoQ10 and other mitochondria-targeted antioxidants, N acetylcysteine (NAC), vitamin C, vitamin E vitamin K1, vitamin B, sodium pyruvate and α-lipoic acid), and NAD precursors (for example, niacinamide).

20. (canceled)

21. A method for:
(a) improving or maintaining skin barrier function;
(b) for preventing and/or treating transepidermal water loss;
(c) for improving skin elasticity and firmness;
(d) for increasing or maintaining skin layer thickness;
(e) for increasing or maintaining the undulation index of dermal epidermal junctions in skin;
(f) for increasing or maintaining skin hydration;
(g) for combating signs of skin aging,
(h) for reducing skin biological aging;
(i) for diminishing fine lines and wrinkles;
(j) for delaying and/or reducing and/or preventing signs of aging selected from the group consisting of skin firmness and/or loss of elasticity, thinning of the skin, wrinkles and fineness. Pattern, dull skin, hyperpigmentation of the skin or hyperpigmentation of pigmentation and pigmentation;
(k) for reduction of skin damage by irradiation, for example UV irradiation-induced skin damage;
(l) for protecting skin from damage caused by radiation, e.g., UV, beta or gamma radiation including during medical treatment for a condition such as a cancer.
(m) for reduction of hair damage by irradiation, for example UV irradiation-induced skin damage;
(n) for protecting hair from damage caused by radiation, e.g., UV, beta or gamma radiation including during medical treatment for a condition such as a cancer;
(o) for improving collagen organization in the skin;
(p) for improving irritation due to dry skin
(q) decreasing sun-induced skin redness; and/or
(r) for protection from sun-induced photoaging,
comprising administering to a subject in need thereof an effective amount of the composition of claim 9.

22. A method for:
(a) maintaining or enhancing hair thickness;
(b) hair follicle cell regeneration;
(c) hair follicle cell survival;
(d) hair stem cell growth and/or regeneration;
(e) hair cell survival;
(f) hair loss prevention,
(g) promoting new hair growth;
(h) scalp health improvement, and/or
(i) hair growth restoration promotion,
comprising administering to a subject in need thereof an effective amount of the composition of claim 9.

23. A method for:
(a) renewal and/or revitalization of skin appearance;
(b) enhancement of skin energy supply;
(c) improvement in skin texture;
(d) enhancement of skin radiance;
(e) skin rejuvenation;
(f) reduction of pore and micro line visibility;
(g) improvement in skin respiration;
(h) support optimal cellular function in skin;
(i) promote/activate skin detoxifying processes;
(j) promote collagen repair, and/or
(k) improve skin stem cell health,
comprising administering to a subject in need thereof an effective amount of the composition of claim 9.

24. A method for:
(a) acne;
(b) contact dermatitis
(c) eczema;
(d) psoriasis;
(e) rosacea,
(f) lupus erythematosus;
(g) warts;
(h) shingles;
(i) hives;
(j) melasma;
(k) liver spots;
(l) lentigo;
(m) skin irritation;
(n) skin infection;
(o) skin inflammation;
(p) seborrheic dermatitis;
(q) Skin fungal infections (for example, candidiasis, athletes' feet);
(r) Skin cancer; and
(s) diaper rash,
comprising administering to a subject in need thereof an effective amount of the composition of claim 9.
